(19) United States
(12) Patent Application Publication
(10) Pub. No.: US 2019/0062736 A1
(43) Pub. Date: Feb. 28, 2019

(54) IN SITU AND IN VIVO ANALYSIS OF CHROMATIN INTERACTIONS BY BIOTINYLATED DCAS9 PROTEIN

(71) Applicant: BOARD OF REGENTS, THE UNIVERSITY OF TEXAS SYSTEM, Austin, TX (US)

(72) Inventors: Xin Liu, Dallas, TX (US); Jian Xu, Plano, TX (US)

(21) Appl. No.: 16/108,307

(22) Filed: Aug. 22, 2018

Related U.S. Application Data
(60) Provisional application No. 62/548,674, filed on Aug. 22, 2017.

(51) Int. Cl. C12N 15/11; C12N 15/113

(52) U.S. Cl. CPC C12N 15/111; C12N 15/113; C12N 2310/20; C07K 14/195; C07K 2319/21; C07K 2319/23; C07K 2319/24; C07K 2319/22

(57) ABSTRACT

The present invention includes a method for detecting or isolating one or more specific genomic target regions and molecules interacting therewith comprising: contacting a recombinant nuclease-deficient Cas9 fusion protein (dCas9) modified to comprise a biotinylation sequence and one or more sequence-specific guide RNAs, with one or more specific genomic DNA targets in cells to form a CRISPR complex; and detecting or isolating the CRISPR complex with a streptavidin or an avidin to detect or isolate the one or more specific genomic target regions and molecules in the CRISPR complex.

### CROSS-REFERENCE TO RELATED APPLICATIONS

This application claims priority to U.S. Provisional Application Ser. No. 62/548,674, filed Aug. 22, 2017, the entire contents of which are incorporated herein by reference.

### STATEMENT OF FEDERALLY FUNDED RESEARCH

This invention was made with government support under grants R01MH102616, K01DK093543, R03DK101665, and R01DK111430 awarded by National Institutes of Health. The government has certain rights in the invention.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Aug. 21, 2018, is named UTSW1093_SL.txt and is 88,941 bytes in size.

### TECHNICAL FIELD OF THE INVENTION

The present invention relates in general to the field of in situ and in vivo analysis of complex chromatin interactions in the Clustered Regularly Interspaced Short Palindromic Repeats (CRISPR) complex.

### BACKGROUND OF THE INVENTION

Without limiting the scope of the invention, its background is described in connection with in situ and in vivo analysis of complex chromatin interactions.

Temporal and tissue-specific gene expression depends on cis-regulatory elements (CREs) and associated trans-acting factors. In contrast to protein-coding genes, a comprehensive understanding of cis-regulatory DNA is very limited. To date, an analysis of the human epigenome has revealed more than one million DNase I hypersensitive sites (DHS), many of which act as transcriptional enhancers (Thurman et al., 2012); however, the regulatory composition of the vast majority of these elements remain unknown largely due to the limitations of the technologies previously employed to study CREs.

Cis-regulatory DNA is bound and interpreted by protein and RNA complexes, and is organized as a 3D structure through long-range chromatin interactions. Identifying the complete composition of a specific CRE in situ can provide unprecedented insight into the mechanisms regulating its activity. However, purifying a small chromatin segment from the cellular milieu represents a major challenge—the protein complexes isolated with the targeted chromatin constitute only a small fraction of the co-purified proteins, most of which are non-specific associations. As such, major challenges have limited the application of existing approaches in purifying a specific genomic locus.

Chromatin immunoprecipitation (ChIP) assays have provided crucial insights into the genome-wide distribution of TFs and histone marks, but it relies on a priori identification of molecular targets, and is confined to examining single TFs. Targeted purification of genomic loci with engineered binding sites has been employed to identify single locus-associated proteins, yet it requires knock-in gene targeting, which remains inefficient. DNA sequence-specific molecules, such as locked nucleic acids (LNAs) (Dejardin and Kingston, 2009) and transcription activator-like (TAL) proteins (Fujita et al., 2013), have been used to enrich large chromatin structures, but these approaches do not enrich for a single genomic locus and cannot be adapted for multiplexed applications. The development of the CRISPR system containing an inactive Cas9 nuclease facilitated sequence-specific enrichment of native genomic regions (Fujita and Fujii, 2013; Waldrip et al., 2014); however, these studies were limited to antibody-based purification. As a result of these limitations, genome-scale specificity and the utility in identifying the cis- and trans-regulatory components were not evaluated.

Thus, a need remains for compositions and methods for improving the understanding of complex chromatin interactions and components of the same.

### SUMMARY OF THE INVENTION

In one embodiment, the present invention includes a method for detecting or isolating one or more specific genomic target regions and molecules interacting therewith comprising: contacting a recombinant nuclease-deficient Cas9 fusion protein (dCas9) modified to comprise a biotinylation sequence and one or more sequence-specific guide RNAs, with one or more specific genomic DNA targets in cells to form a CRISPR complex; and detecting or isolating the CRISPR complex with a streptavidin or an avidin to detect or isolate the one or more specific genomic target regions and molecules in the CRISPR complex. In one aspect, the method further comprises fragmenting the genomic DNA in the cells under conditions in which the genomic DNA and molecules interacting therewith are maintained in the CRISPR complex. In another aspect, the method further comprises isolating the CRISPR complex after fragmentation of the genomic DNA. In another aspect, the method further comprises identifying one or more of proteins, peptides, nucleic acids, genomic DNA, or molecules in the CRISPR complex. In another aspect, the one or more sequence-specific guide RNAs are programmable sequence-specific guide RNAs. In another aspect, the recombinant biotinylated nuclease-deficient Cas9 fusion protein has been modified to comprise a biotinylation sequence that is biotinylatable in vivo. In another aspect, the recombinant biotinylated nuclease-deficient Cas9 is a fusion protein with an isolatable peptide tag at the N- or C-terminus, or other regions of the dCas9 protein. In another aspect, the isolatable peptide tags are selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both. In another aspect, the method further comprises detecting the CRISPR complex in situ with the streptavidin or avidin bound to a detectable label. In another aspect, the biotinylated dCas9 fusion protein is biotinylated in vivo by BirA enzyme or endogenous biotin ligases in prokaryotic cells, eukaryotic cells, or both. In another aspect, the dCas9 fusion protein is bound with the streptavidin or avidin that has been conjugated to a detectable label selected from at least one of an electrochemiluminescence label, an enzyme label, a fluorophore, a latex particle, a magnetic particle, a radioactive element, a phosphorescent dye, a dye, a gold, silver, or selenium particle, or a ruthenium or osmium metal chelate. In another aspect, the streptavidin or avidin is bound to a solid support, a chip, a substrate, a column, a well, or beads. In another aspect, the method further comprises performing a chemical treatment that maintains the interaction of genomic DNA and molecules interacting therewith in the CRISPR complex. In another aspect, the recombinant nuclease-deficient Cas9 fusion protein is SEQ ID NO:334. In another aspect, the method further comprises expressing in the cells a biotin ligase capable of biotinylating the recombinant nuclease-deficient Cas9 fusion protein. In another aspect, the method further comprises identifying nucleic acids, peptides, proteins, by at least one of mass spectrometry (MS)-based proteomics, MS-MS, MALDI, MALDI-TOF, multiplex proteomic identification, immunoblot, ELISA, nucleotide sequence analysis, microarray analysis, or PCR. In another aspect, the method further comprises capturing in situ one or more locus-specific chromatin interactions by biotinylated dCas9 fusion protein. In another aspect, the method further comprises using biotinylated dCas9-mediated capture of the binding cluster at or around the sequence-specific guide RNA. In another aspect, the method further comprises identifying cis-regulatory elements (CRE)-associated protein complexes to identify proteins or nucleic acids of the CRISPR complex. In another aspect, the method further comprises using the CRISPR complex for CRISPR affinity purification in situ of regulatory elements (CAPTURE)-proteomics to identify known and new regulators of at least one of genes, promoters, or enhancers by: cross-linking the CRISPR complex, fragmenting the complex, dCas9 fusion protein affinity purification, and sequencing the nucleic acids isolated therewith, western blot, or peptide digestion with multiplex identification by proteomic profiling. In another aspect, the method further comprises using CAPTURE-3C-seq to identify locus-specific long-range DNA interactions by crosslinking of the CRISPR complex, enzymatic digestion of nucleic acids, proximity ligation of the nucleic acids, fragmentation of the genomic DNA, dCas9 fusion protein affinity purification, and pair-end sequencing to identify tethered long-range interactions. In another aspect, the enzymatic digestion is by at least one of AatI, AatII, AauI, Acc113I, Acc16I, Acc65I, AccB1I, AccB7I, AccBSI, AccI, AccII, AccIII, AceIII, AciI, AclI, AclNI, AclWI, AcsI, AcyI, AdeI, AfaI, AfeI, AflII, AflIII, AgeI, AhaIII, AhdI, AluI, Alw21I, Alw26I, Alw44I, AlwI, AlwNI, Ama87I, AocI, Aor51HI, ApaBI, Apal, ApaLI, ApoI, AscI, Asel, AsiAI, AsnI, Asp700I, Asp718I, AspEI, AspHI, AspI, AspLEI, AspS9I, AsuC2I, AsuHPI, AsuI, AsuII, AsuNHI, AvaI, AvaII, AvaIII, AviII, AvrII, AxyI, BaeI, BalI, BamHI, BanI, BanII, BanIII, BbeI, BbiII, BbrPI, BbsI, BbuI, Bbv12I, BbvCI, BbvI, BbvII, BccI, Bce83I, BcefI, BcgI, BciVI, BclI, BcnI, BcoI, BcuI, BetI, BfaI, BfiI, BfmI, BfrI, BglI, BglII, BinI, BlnI, BlpI, Bme18I, BmgI, BmrI, BmyI, BpiI, BplI, BpmI, Bpu10I, Bpu1102I, Bpu14I, BpuAI, Bsa29I, BsaAI, BsaBI, BsaHI, BsaI, BsaJI, BsaMI, BsaOI, BsaWI, BsaXI, BsbI, Bsc4I, BscBI, BscCI, BscFI, BscGI, BscI, Bse118I, Bse1I, Bse21I, Bse3DI, Bse8I, BseAI, BseCI, BseDI, BseGI, BseLI, BseMII, BseNI, BsePI, BseRI, BseX3I, BsgI, Bsh1236I, Bsh1285I, Bsh1365I, BshI, BshNI, BsiBI, BsiCI, BsiEI, BsiHKAI, BsiI, BsiLI, BsiMI, BsiQI, BsiSI, BsiWI, BsiXI, BsiYI, BsiZI, BslI, BsmAI, BsmBI, BsmFI, BsmI, BsoBI, Bsp106I, Bsp119I, Bsp120I, Bsp1286I, Bsp13I, Bsp1407I, Bsp143I, Bsp143II, Bsp1720I, Bsp19I, Bsp24I, Bsp68I, BspA2I, BspCI, BspDI, BspEI, BspGI, BspHI, BspLI, BspLU11I, BspMI, BspMII, BspTI, BspXI, BsrBI, BsrBRI, BsrDI, BsrFI, BsrGI, BsrI, BsrSI, BssAI, BssHII, BssKI, BssNAI, BssSI, BssT1I, Bst1107I, Bst2BI, Bst2UI, Bst4CI, Bst71I, Bst98I, BstACI, BstAPI, BstBAI, BstBI, BstDEI, BstDSI, BstEII, BstF5I, BstH2I, BstHPI, BstMCI, BstNI, BstNSI, BstOI, BstPI, BstSFI, BstSNI, BstUI, BstX2I, BstXI, BstYI, BstZ17, BstZI, Bsu15I, Bsu36I, Bsu6I, BsuRI, BtgI, BtsI, Cac8I, CauII, CbiI, CciNI, CelII, CfoI, Cfr10I, Cfr13I, Cfr42I, Cfr9I, CfrI, CjeI, CjePI, ClaI, CpoI, Csp45I, Csp6I, CspI, CviJI, CviRI, CvnI, DdeI, DpnI, DpnII, DraI, DraII, DraIII, DrdI, DrdII, DsaI, DseDI, EaeI, EagI, Eam1104I, Eaml 11051, EarI, EciI, Ec136II, EclHKI, EclXI, Eco105I, Eco130I, Eco147I, Eco24I, Eco255I, Eco31I, Eco32I, Eco47I, Eco47III, Eco52I, Eco57I, Eco64I, Eco72I, Eco81I, Eco88I, Eco91I, EcoICRI, EcoNI, EcoO109I, EcoO65I, EcoRI, EcoRII, EcoRV, EcoT14I, EcoT22I, EcoT38I, EgeI, EheI, ErhI, Esp1396I, Esp3I, EspI, FauI, FauNDI, FbaI, FinI, Fnu4HI, FnuDUII, FokI, FriOI, FseI, Fsp4HI, FspI, GdiII, GsuI, HaeI, HaeII, HaeIII, HaeIV, HapII, HgaI, HgiAI, HgiCI, HgiEI, HgiEII, HgiJII, HhaI, Hin1I, Hin2I, Hin4I, Hin6I, HincII, HindII, HindIII, HinfI, HinP1I, HpaI, HpaII, HphI, Hsp92I, Hsp92II, HspAI, ItaI, KasI, Kpn2I, KpnI, Ksp22I, Ksp632I, KspAI, KspI, Kzo9I, LspI, MaeI, MaeII, MaeIII, MamI, MbiI, MboI, MboII, McrI, MfeI, MflI, MlsI, MluI, MluNI, Mly113I, MmeI, MnlI, Mph1103I, MroI, MroNI, MroXI, MscI, MseI, MslI, Msp171, MspA1I, MspCI, MspI, MspR9I, MstI, MunI, Mva1269I, MvaI, MvnI, MwoI, NaeI, NarI, NciI, Ncol, NdeI, NdeII, NgoAIV, NgoMIV (previously known as NgoMI), NheI, NlaIII, NlaIV, NotI, NruGI, NruI, NsbI, NsiI, NspBII, NspI, NspV, PacI, PaeI, PaeR7I, PagI, PalI, PauI, Pfl1108I, Pfl23II, PflFI, PflMI, PinAI, Ple19I, PleI, PmaCI, Pme55I, PmeI, PmlI, Ppu10I, PpuMI, PshAI, PshBI, Psp124BI, Psp1406I, Psp5II, PspAI, PspEI, PspLI, PspN4I, PspOMI, PspPPI, PstI, PvuI, PvuII, RcaI, RleAI, RsaI, RsrII, SacI, SacII, SalI, SanDI, SapI, Sau3AI, Sau96I, SauI, SbfI, ScaI, SchI, ScrFI, SdaI, SduI, SecI, SexAI, SfaNI, SfcI, SfeI, SfiI, SfoI, Sfr274I, Sfr303I, SfuI, SgfI, SgrAI, SimI, SinI, SmaI, SmiI, SmlII, SnaBI, SnaI, SpeI, SphI, SplI, SrfI, Sse8387I, Sse86471, Sse9I, SseBI, SspBI, SspI, SstI, SstII, StuI, StyI, SunI, SwaI, TaiI, TaqI, TaqII, TatI, TauI, TfiI, ThaI, TruII, Tru9I, TscI, TseI, Tsp45I, Tsp4CI, Tsp509I, TspEI, TspRI, Tth111I, Tth111II, TthHB8I, UbaDI, UbaEI, UbaLI, UbaOI, Van91I, Vha4641, VneI, VspI; XagI, XbaI, XcmI, XhoI, XhoII, XmaCI, XmaI, XmaIII XmnI, Zsp2I, Tn5 transposases, DNase, or micrococcal nuclease (MNase). In another aspect, the method further comprises using biotinylated dCas9-mediated in situ capture of a disease-associated cis-regulatory elements (CRE) to measure cis-transcription factors, RNA complexes, and long-range DNA interactions that contribute to the disease phenotypes. In another aspect, the method further comprises using multiplexed CAPTURE of developmentally regulated super-enhancers during differentiation. In another aspect, the method further comprises multiplexed CAPTURE using 2, 3, 4, 5, 6, 7, 8, 9, 10, 15, 20, 25, 30, 35, 40, 45, 50, 60, 70, 75, 80, 90, 100, 200, 300, 400, 500, 600, 700, 800, 900, 1,000, 2,000, 3,000, 4000, 5,000, 6,000, 7,000, 8,000, 9,000, 10,000, or more sgRNAs in a pool to target multiple genomic regions, including multiple cis-elements at the same enhancer cluster or multiple independent enhancers. In another aspect, the method further comprises detecting the CRISPR complex in situ.

In another embodiment, the present invention includes a method for identifying one or more specific genomic target regions and molecules interacting therewith comprising: contacting a recombinant nuclease-deficient Cas9 fusion protein (dCas9 fusion protein) modified to comprise a biotinylation sequence and one or more sequence-specific guide RNAs to the one or more specific genomic DNA targets in cells to form a CRISPR complex; in vivo biotinylating the dCas9 fusion protein with a biotin ligase; fragmenting the genomic DNA around the CRISPR complex; isolating the CRISPR complex with a streptavidin or an avidin; and determining an identity of one or more proteins, DNAs, or RNAs in the CRISPR complex. In one aspect, the method further comprises fragmenting the genomic DNA in the cells under conditions in which the genomic DNA and molecules interacting therewith are maintained in the CRISPR complex. In another aspect, the one or more sequence-specific guide RNAs are programmable sequence-specific guide RNAs (sgRNAs). In another aspect, the recombinant biotinylated nuclease-deficient Cas9 is a fusion protein with an isolatable peptide tag at the N-,C-terminus or other regions of the dCas9 protein selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both. In another aspect, the dCas9 fusion protein is bound with the streptavidin or avidin that has been conjugated to a detectable label selected from at least one of an electrochemiluminescence label, an enzyme label, a fluorophore, a latex particle, a magnetic particle, a radioactive element, a phosphorescent dye, a dye, a gold, silver, or selenium particle, or a ruthenium or osmium metal chelate. In another aspect, the streptavidin or avidin is bound to a solid support, a chip, a substrate, a column, a well, or beads. In another aspect, the method further comprises performing a chemical treatment that maintains the interaction of genomic DNA and molecules interacting therewith in the CRISPR complex. In another aspect, the recombinant nuclease-deficient Cas9 fusion protein is SEQ ID NO:334. In another aspect, the method further comprises expressing in the cells a biotin ligase capable of biotinylating the recombinant nuclease-deficient Cas9 fusion protein. In another aspect, the method further comprises identifying nucleic acids, peptides, proteins, by at least one of mass spectrometry (MS)-based proteomics, MS-MS, MALDI, MALDI-TOF, multiplex proteomic identification, immunoblot, ELISA, nucleotide sequence analysis, microarray analysis, or PCR. In another aspect, the method further comprises capturing in situ one or more locus-specific chromatin interactions by biotinylated dCas9 fusion protein. In another aspect, the method further comprises using biotinylated dCas9-mediated capture of the binding cluster at or about the sequence-specific guide RNA. In another aspect, the method further comprises identifying Cis-regulatory elements (CRE)-associated protein complexes to identify proteins or nucleic acids of the CRISPR complex. In another aspect, the method further comprises using the CRISPR complex for CRISPR affinity purification in situ of regulatory elements (CAPTURE)-proteomics to identify known and new regulators of at least one of genes, promoters, or enhancers. In another aspect, the method further comprises using CAPTURE-3C-seq to identify locus-specific long-range DNA interactions. In another aspect, the method further comprises using biotinylated dCas9-mediated in situ capture of a disease-associated CRE. In another aspect, the method further comprises using multiplexed CAPTURE of developmentally regulated super-enhancers during differentiation. In another aspect, the method further comprises using multiplexed CAPTURE using 2, 3, 4, 5, 6, 7, 8, 9, 10, 15, 20, 25, 30, 35, 40, 45, 50, 60, 70, 75, 80, 90, 100, 200, 300, 400, 500, 600, 700, 800, 900, 1,000, 2,000, 3,000, 4000, 5,000, 6,000, 7,000, 8,000, 9,000, 10,000, or more sgRNAs in a pool to target multiple genomic regions, including multiple cis-elements at the same enhancer cluster or multiple independent enhancers. In another aspect, the method further comprises identifying significantly enriching molecular interactions at one or more genomic targets by comparing the molecules in the CRISPR complex when compared to one or more negative controls. In another aspect, the negative controls include one or more of the following: cells expressing biotin ligase (BirA) only, cells expression BirA and dCas9 fusion protein, cells expression BirA, dCas9 and the non-targeting sgRNA (sgGal4), and cells expression BirA, dCas9, one or more sequence-specific sgRNAs, and knockout of the sgRNA targeting sequences in the genome.

In another embodiment, the present invention includes a method for identifying one or more long-range DNA interactions (or looping) with a CRISPR complex comprising: contacting a recombinant nuclease-deficient Cas9 fusion protein (dCas9 fusion protein) modified to comprise a biotinylation sequence or another isolatable tag and one or more sequence-specific guide RNAs to the one or more specific genomic DNA targets in cells to form a CRISPR complex; in vivo biotinylating the dCas9 fusion protein with a biotin ligase; enzymatically digesting genomic DNA with a restriction enzyme or other nucleases; proximity ligating one or more nucleic acids in the CRISPR complex; isolating the CRISPR complex by affinity purification with a streptavidin or an avidin; and pair-end sequencing to identify tethered long-range interactions in the CRISPR complex. In one aspect, restriction enzyme or nuclease is selected from at least one of: AatI, AatII, AauI, Acc113I, Acc16I, Acc65I, AccB1I, AccB7I, AccBSI, AccI, AccII, AccIII, AceIII, AciI, AclI, AclNI, AclWI, AcsI, AcyI, AdeI, AfaI, AfeI, AflII, AflIII, AgeI, AhaIII, AhdI, AluI, Alw21I, Alw26I, Alw44I, AlwI, AlwNI, Ama87I, AocI, Aor51HI, ApaBI, Apal, ApaLI, ApoI, AscI, Asel, AsiAI, AsnI, Asp700I, Asp718I, AspEI, AspHI, AspI, AspLEI, AspS9I, AsuC2I, AsuHPI, AsuI, AsuII, AsuNHI, AvaI, AvaII, AvaIII, AviII, AvrII, AxyI, BaeI, BalI, BamHI, BanI, BanII, BanIII, BbeI, BbiII, BbrPI, BbsI, BbuI, Bbv12I, BbvCI, BbvI, BbvII, BccI, Bce83I, BcefI, BcgI, BciVI, BclI, BcnI, BcoI, BcuI, BetI, BfaI, BfiI, BfmI, BfrI, BglI, BglII, BinI, BlnI, BlpI, Bme18I, BmgI, BmrI, BmyI, BpiI, BplI, BpmI, Bpu10I, Bpu11021, Bpu14I, BpuAI, Bsa29I, BsaAI, BsaBI, BsaHI, BsaI, BsaJI, BsaMI, BsaOI, BsaWI, BsaXI, BsbI, Bsc4I, BscBI, BscCI, BscFI, BscGI, BscI, Bse118I, Bse1I, Bse21I, Bse3DI, Bse8I, BseAI, BseCI, BseDI, BseGI, BseLI, BseMII, BseNI, BsePI, BseRI, BseX3I, BsgI, Bsh1236I, Bsh1285I, Bsh1365I, BshI, BshNI, BsiBI, BsiCI, BsiEI, BsiHKAI, BsiI, BsiLI, BsiMI, BsiQI, BsiSI, BsiWI, BsiXI, BsiYI, BsiZI, BslI, BsmAI, BsmBI, BsmFI, BsmI, BsoBI, Bsp106I, Bsp119I, Bsp120I, Bsp1286I, Bsp13I, Bsp1407I, Bsp143I, Bsp143II, Bsp1720I, Bsp19I, Bsp24I, Bsp68I, BspA2I, BspCI, BspDI, BspEI, BspGI, BspHI, BspLI, BspLU11I, BspMI, BspMII, BspTI, BspXI, BsrBI, BsrBRI, BsrDI, BsrFI, BsrGI, BsrI, BsrSI, BssAI, BssHII, BssKI, BssNAI, BssSI, BssT1I, Bst1107I, Bst2BI, Bst2UI, Bst4CI, Bst71I, Bst98I, BstACI, BstAPI, BstBAI, BstBI, BstDEI, BstDSI, BstEII, BstF5I, BstH2I, BstHPI, BstMCI, BstNI, BstNSI, BstOI, BstPI, BstSFI, BstSNI, BstUI, BstX2I, BstXI, BstYI, BstZ17, BstZI, Bsu15I, Bsu36I, Bsu6I, BsuRI, BtgI, BtsI, Cac8I, CauII, CbiI, CciNI, CelII, CfoI, Cfr10I, Cfr13I, Cfr42I, Cfr9I, CfrI, CjeI, CjePI, ClaI, CpoI, Csp45I, Csp6I, CspI, CviJI, CviRI, CvnI, DdeI, DpnI, DpnII, DraI, DraII, DraIII, DrdI, DrdII, DsaI, DseDI, EaeI, EagI, Eam1104I, Eaml 11051, EarI, EciI, Ec136II, EclHKI, EclXI, Eco105I, Eco130I, Eco147I, Eco24I, Eco255I, Eco31I, Eco32I, Eco47I, Eco47III, Eco52I, Eco57I, Eco64I, Eco72I, Eco81I, Eco88I, Eco91I, EcoICRI, EcoNI, EcoO109I, EcoO65I, EcoRI, EcoRII, EcoRV, EcoT14I, EcoT22I, EcoT38I, EgeI, EheI, ErhI, Esp1396I, Esp3I, EspI, FauI, FauNDI, FbaI, FinI, Fnu4HI, FnuDUII, FokI, FriOI, FseI, Fsp4HI, FspI, GdiII, GsuI, HaeI, HaeII, HaeIII, HaeIV, HapII, HgaI, HgiAI, HgiCI, HgiEI, HgiEII, HgiJII, HhaI, Hin1I, Hin2I, Hin4I, Hin6I, HincII, HindII, HindIII, HinfI, HinP1I, HpaI, HpaII, HphI, Hsp92I, Hsp92II, HspAI, ItaI, KasI, Kpn2I, KpnI, Ksp22I, Ksp632I, KspAI, KspI, Kzo9I, LspI, MaeI, MaeII, MaeIII, MamI, MbiI, MboI, MboII, McrI, MfeI, MflI, MlsI, MluI, MluNI, Mly113I, MmeI, MnlI, Mph1103I, MroI, MroNI, MroXI, MscI, MseI, MslI, Msp171, MspA1I, MspCI, MspI, MspR9I, MstI, MunI, Mva1269I, MvaI, MvnI, MwoI, NaeI, NarI, NciI, Ncol, NdeI, NdeII, NgoAIV, NgoMIV (previously known as NgoMI), NheI, NlaIII, NlaIV, NotI, NruGI, NruI, NsbI, NsiI, NspBII, NspI, NspV, PacI, PaeI, PaeR7I, PagI, PalI, PauI, Pfl1108I, Pfl23II, PflFI, PflMI, PinAI, Ple19I, PleI, PmaCI, Pme55I, PmeI, PmlI, Ppu10I, PpuMI, PshAI, PshBI, Psp124BI, Psp1406I, Psp5II, PspAI, PspEI, PspLI, PspN4I, PspOMI, PspPPI, PstI, PvuI, PvuII, RcaI, RleAI, RsaI, RsrII, SacI, SacII, SalII, SanDI, SapI, Sau3AI, Sau96I, SauI, SbfI, ScaI, SchI, ScrFI, SdaI, SduI, SecI, SexAI, SfaNI, SfcI, SfeI, SfiI, SfoI, Sfr274I, Sfr303I, SfuI, SgfI, SgrAI, SimI, SinI, SmaI, SmiI, SmlII, SnaBI, SnaI, SpeI, SphI, SplI, SrfI, Sse8387I, Sse8647I, Sse9I, SseBI, SspBI, SspI, SstI, SstII, StuI, StyI, SunI, SwaI, TaiI, TaqI, TaqII, TatI, TauI, TfiI, ThaI, TruII, Tru9I, TscI, TseI, Tsp45I, Tsp4CI, Tsp509I, TspEI, TspRI, Tth111I, Tth111II, TthHB8I, UbaDI, UbaEI, UbaLI, UbaOI, Van91I, Vha4641, VneI, VspI; XagI, XbaI, XcmI, XhoI, XhoII, XmaCI, XmaI, XmaIII XmnI, Zsp2I, Tn5 transposases, DNase, or micrococcal nuclease (MNase). In one aspect, the method further comprises the step of crosslinking the CRISPR complex. In another aspect, the method further comprises fragmenting the genomic DNA after isolating the CRISPR complex. In another aspect, the step of affinity purification of the CRISPR complex is performed using a isolatable tag selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both.

In yet another embodiment, the present invention includes a nucleic acid vector encoding a recombinant nuclease-deficient Cas9 fusion protein (dCas9) modified to comprise a biotinylation sequence and a tag sequence. In one aspect, the nucleic acid vector further comprises a biotin ligase gene. In another aspect, the tag sequence is selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both. In another aspect, the recombinant dCas9 with the biotinylation site has nucleic acid sequence SEQ ID NO:333.

In yet another embodiment, the present invention includes a protein comprising a recombinant nuclease-deficient Cas9 fusion protein (dCas9 fusion protein) modified to comprise a biotinylation sequence and a tag sequence. In one aspect, the tag sequence is at the N- or C-terminus, or in other regions of the dCas9 protein. In another aspect, the tag sequence is selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in both prokaryotic and eukaryotic cells. In another aspect, the recombinant dCas9 fusion protein is bound to a solid support, a chip, a substrate, a column, a well, or beads by streptavidin or avidin. In another aspect, the recombinant dCas9 with the biotinylation site has amino acid sequence SEQ ID NO:334.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. As the color drawings are being filed electronically via EFS-Web, only one set of the drawings is submitted.

For a more complete understanding of the features and advantages of the present invention, reference is now made to the detailed description of the invention along with the accompanying figures and in which:

FIGS. 1A to 1G show in Situ Capture of Locus-Specific Chromatin Interactions by Biotinylated dCas9. (FIG. 1A) Schematic of dCas9-mediated capture of chromatin interactions. (FIG. 1B) The three components of the CAPTURE system: a FB-dCas9, a biotin ligase BirA, and target-specific sgRNAs. (FIG. 1C) Schematic of dCas9-mediated capture of human telomeres. (FIG. 1D) Labeling of human telomeres in MCF7 cells. Scale bar, 5 μm. (FIG. 1E) qPCR analysis shows significant enrichment of telomere DNA. Results are mean±SEM of three experiments and analyzed by two-tailed t-test. **P<0.01. (FIG. 1F) Western blot shows enrichment of TERF2 in sgTelomere-expressing but not control K562 cells with dCas9 alone (no sgRNA) or the non-targeting sgGal4. (FIG. 1G) iTRAQ-based proteomics analysis of telomere-associated proteins. Representative proteins and the mean iTRAQ ratios are shown. See also Table 3.

FIGS. 2A to 2G show biotinylated dCas9-Mediated Capture of the β-Globin Cluster. (FIG. 2A) Schematic of CAPTURE-ChIP-seq. (FIG. 2B) Density maps are shown for CAPTURE-ChIP-seq at the β-globin cluster (chr11:5,222,500-5,323,700; hg19) in K562 cells, together with DHS and H3K27ac ChIP-seq profiles. Two independent sgRNAs (sg1 and sg2) or replicate experiments (rep1 and rep2) are shown. Cells expressing dCas9 only (no sgRNA) or dCas9 with sgGal4 were analyzed as controls. (FIG. 2C) Genome-wide analysis of dCas9 binding in cells expressing two sgRNAs (sg1 and sg2) for HS2 or HBG. Data points for the sgRNA target regions and the predicted off-targets are shown as green, red and orange, respectively. The x- and y-axis denote the mean normalized read counts from N=2 to 5 CAPTURE-ChIP-seq experiments. (FIGS. 2D-2F) Genome-wide differential analysis of dCas9 binding in cells expressing sgHS2, sgHBG, or sgHS1-5 versus sgGal4. Data points for the sgRNA target regions and the predicted off-targets are shown as green and red, respectively. N=5, 4, 6 and 4 CAPTURE-ChIP-seq experiments for sgHS2, sgHBG, sgHS1-5 and sgGal4, respectively. (FIG. 2G) RNA-seq analysis was performed in cells expressing dCas9 with sgHS2, sgHBG, sgHS1-5, sgGal4 or WT K562 cells. The Pearson correlation coefficient (R) value is shown. See also FIG. 8, Tables 1 and 2.

FIGS. 3A to 3E show CAPTURE-Proteomics Identify β-Globin CRE-Associated Protein Complexes. (FIG. 3A) Schematic of CAPTURE-Proteomics. (FIG. 3B) Western blot analysis of captured proteins in sgHS1-5 or sgGal4-expressing K562 cells. (FIG. 3C) Schematic of the β-globin cluster and sgRNAs used for CAPTURE-Proteomics. (FIG. 3D) CAPTURE-Proteomics identified β-globin CRE-associated proteins. Volcano plots are shown for the iTRAQ proteomics of purifications in sgHS2, sgHBG or sgHBB versus sgGal4-expressing cells. Relative protein levels in target-specific sgRNAs versus sgGal4 are plotted on the x-axis as mean log 2 iTRAQ ratios across N replicate experiments. Negative log 10 transformed P values are plotted on the y-axis. Significantly enriched proteins (P≤0.05; iTRAQ ratio ≥1.5) are denoted by black dots, all others by grey dots. Dotted lines indicate 1.5-fold ratio (x-axis) and P value of 0.05 (y-axis). Representative chromatin-regulating proteins are denoted by red arrowheads. Representative proteins with iTRAQ ratio ≥1.5 and P >0.05 are denoted by blue arrowheads. (FIG. 3E) Connectivity network of CAPTURE-Proteomics-identified proteins converged by β-globin CREs. The connectivity was built using interactions (grey lines) between proteins and CREs. Colored nodes denote proteins enriched at single or multiple CREs. Size of the circles denotes the frequency of interactions. Inset tables show the lists of representative proteins associated with the β-globin promoters (red), enhancers (blue) or both (green). See also FIGS. 9 and 10.

FIGS. 4A to 4H show CAPTURE-Proteomics Identify Known and New Regulators of β-Globin Genes and Erythroid Enhancers. (FIG. 4A) ChIP-seq analysis of the identified regulators in K562 cells. (FIG. 4B) RNAi screen of the identified regulators in human primary erythroid cells. Data are plotted as log 2 (fold change) of the β-globin mRNA in each shRNA experiment relative to the non-targeting shNT control. Genes are ranked based on the changes in HBE1, HBG or HBB expression. shRNAs against BCL11A and KLF1 were analyzed as controls. Results are mean±SEM of all shRNAs for each gene from four experiments. (FIG. 4C) Genome-wide distribution of NUP98 and NUP153 ChIP-seq peaks in promoters (−2 kb to 1 kb of TSS), exons, intragenic and intergenic regions. (FIG. 4D) NUP98 and NUP153 associate with erythroid SEs. SEs were identified by ROSE (Whyte et al., 2013) using the H3K27ac ChIP-seq signal. (FIG. 4E) Representative SE loci co-occupied by NUP98 and NUP153. DHS, ChIP-seq, and chromatin state (ChromHMM) data are shown. Red bars denote the annotated SEs. (FIG. 4F) NUP98 and NUP153-associated genes show significantly higher mRNA expression. Boxes show median of the data and quartiles, and whiskers extend to 1.5× of the interquartile range. P values were calculated by a two-side t-test. (FIG. 4G) Enriched gene ontology (GO) terms associated with NUP98 or NUP153 occupied regions. (FIG. 4H) Motif analysis of NUP98 or NUP153 binding sites.

FIGS. 5A to 5F show CAPTURE-3C-seq Identifies Locus-Specific Long-Range DNA Interactions. (FIG. 5A) Schematic of CAPTURE-3C-seq. (FIG. 5B) Browser view of the long-range interactions at HS3 (chr11:5,222,500-5,323,700; hg19) is shown. Contact profiles including the density map, interactions (or loops) and PETs are shown. The statistical significance of interactions was determined by the Bayes factor (BF) and indicated by the color scale bars. ChIA-PET, DHS, ChIP-seq, RNA-seq, and ChromHMM data are shown. (FIG. 5C) Circlet plots of the long-range interactions are shown. The numbers of identified inter- (blue lines) and intra-chromosomal (purple lines) interactions are shown. (FIG. 5D) Browser view of the long-range interactions at the active HBG (green shaded lines) and the repressed HBB promoters (red shaded lines) is shown. (FIG. 5E) The fraction of identified interactions relative to the total PETs at each captured region is shown. Results are mean±SEM of two or three experiments and analyzed by a two-sided t-test. *P<0.05; ***P<0.001. (FIG. 5F) KO of de novo CREs impaired the expression of β-globin genes. The log 2 (fold change) of the mRNA expression in KO versus WT cells are shown. Each circle denotes an independent single-cell-derived KO clone. A diagram depicting the upstream (UpE1, UpE2 and UpE3) and downstream (DnE1, DnE2 and DnE3) CREs is shown on the top. Results are mean±SEM of independent clones and analyzed by a two-sided t-test. *P<0.05, **P<0.01, ***P<0.001. See also FIGS. 11, 12, and 13.

FIGS. 6A to 6H show biotinylated dCas9-Mediated In Situ Capture of A Disease-Associated CRE. (FIG. 6A) Schematic of the 3.5 kb intergenic element (chr11:5,255,859-5,259,368; hg19) along with the deletions mapped in prior studies. (FIG. 6B) Genome-wide specificity of sgHBD-1kb was measured by CAPTURE-ChIP-seq. N=2 and 4 experiments for sgHBD-1kb and sgGal4. (FIG. 6C) Browser view of the long-range interactions at HBD-1kb (red shaded lines) is shown. (FIG. 6D) Circlet plot of the long-range interactions at HBD-1kb is shown. (FIG. 6E) HBD-1kb KO impaired the expression of β-globin genes. Results are mean±SEM of independent KO clones and analyzed by a two-sided t-test. *P<0.05, **P<0.01. (FIG. 6F) HBD-1kb KO led to altered chromatin accessibility and long-range interactions. Results from three ATAC-seq experiments in WT or KO cells are shown. Regions showing increased or decreased ATAC-seq signals in KO relative to WT cells (KO-WT) are depicted in green and red, respectively. HS3 or 3′HS1-mediated long-range interactions were determined by CAPTURE-3C-seq. (FIG. 6G) CAPTURE-Proteomics identified HBD-1kb-associated proteins. Volcano plot is shown for the iTRAQ proteomics of purifications in sgHBD-1kb versus sgGal4-expressing cells. (FIG. 6H) The model of composition-based organization of the β-globin cluster. Top: a previously described model depicting an active chromatin hub (ACH) formed through spatial organization of β-globin CREs (Palstra et al., 2003; Tolhuis et al., 2002). Middle: two-dimensional representation of the long-range DNA interactions (purple lines) identified at HS3 and the HBG1-HBD intergenic CREs (yellow square) by CAPTURE. Bottom: a refined model depicting the composition-based spatial and hierarchical organization of the β-globin CREs. See also FIG. 14, Tables 4 and 5.

FIGS. 7A to 7E show multiplexed CAPTURE of Developmentally Regulated SEs during Differentiation. (FIG. 7A) Schematic of site-specific knock-in of tetracycline-inducible FB-dCas9-EGFP and BirA. (FIG. 7B) Dox-inducible expression of dCas9 and BirA proteins was confirmed by Western blot in two independent knock-in ESC lines. (FIG. 7C) Schematic of multiplexed CAPTURE of ESC-specific SEs in ESCs and EBs. (FIG. 7D) Differentiated EBs were characterized by downregulation of ESC-associated genes (Oct4, Sox2, Esrrb and Utf1) and upregulation of differentiation-associated genes (Vim, Gata4 and Gata6). Results are mean±SEM of 3 or 4 experiments and analyzed by a two-sided t-test. **P<0.01, ***P<0.001. (FIG. 7E) Browser view of SE-associated long-range interactions captured by CAPTURE-3C-seq in ESCs and EBs. Regions showing increased or decreased ATAC-seq or H3K27ac ChIP-seq signals in EBs relative to ESCs (EB-ESC) are depicted in red and blue, respectively. Red bars denote the annotated SEs. Dashed lines denote the alternative TSS of transcript variants for Oct4 (Pou5f1) and Esrrb.

FIGS. 8A to 8G show Genome-Wide Enrichment and Specificity of dCas9-Mediated CAPTURE, related to FIG. 2. (FIG. 8A) CAPTURE-ChIP-seq markedly improved the on-target enrichment compared to antibody-based ChIP-seq. A schematic of the comparison at the captured HS2 enhancer and HBG promoters is shown on the top. The density maps are shown for CAPTURE-ChIP-seq, Cas9 or FLAG antibody-based ChIP-seq, respectively. The y-axis denotes the normalized ChIP-seq intensity as reads per kilobases per million reads (RPKM). (FIG. 8B) The fractions (%) of sgRNA on-target reads were significantly higher in CAPTURE-ChIP-seq than in Cas9 or FLAG antibody-based ChIP-seq. The fold increases in the % of on-target reads at sgHS2 or sgHBG targeted regions in the top 10, 50 or 100 ChIP-seq peaks in CAPTURE-ChIP-seq versus antibody-based ChIP-seq are shown. (FIG. 8C) CAPTURE-ChIP-seq displayed significantly less off-targets compared to antibody-based ChIP-seq. Scatter plots show the genome-wide differential analysis of dCas9 binding at sgHS2 or sgHBG targeted regions by CAPTURE-ChIP-seq, Cas9 or FLAG antibody-based ChIP-seq. Data points for the sgRNA target regions and predicted off-targets are shown as green and red, respectively. Other enriched ChIP-seq peaks are shown as grey. The x- and y-axis denote the mean normalized read counts from N=2 independent CAPTURE-ChIP-seq. (FIG. 8D) Genome-wide differential analysis of dCas9 binding in cells expressing two or three independent sgRNAs (sg1, sg2 and sg3) for sgHS1, sgHS3, sgHS4, sgHS5 or sgHBB targeted regions. Data points for the sgRNA target regions and the predicted off-targets for each sgRNA are shown as green, red and orange, respectively. The x- and y-axis denote the mean normalized read counts from N=2 or 3 independent CAPTURE-ChIP-seq. (FIG. 8E) Genome-wide differential analysis of dCas9 binding in cells expressing sgHS1, sgHS3, sgHS4, sgHS5, sgHBB, or sg3′HS1 versus the non-targeting sgGal4. Data points for the sgRNA target regions and the predicted off-targets are shown as green and red, respectively. N=2 to 4 independent ChIP-seq experiments. (FIG. 8F) Genome-wide differential gene expression analysis was performed using RNA-seq in K562 cells expressing dCas9 with sgHS2, sgHBG, sgHS1-5, the non-targeting sgGal4 or the wild-type (WT) cells. The β-like globin genes are indicated by colored data points. The Pearson correlation coefficient (R) value is calculated for each comparison (N=2 or 3 independent RNA-seq experiments). (FIG. 8G) Expression of β-globin mRNAs remained unchanged in K562 cells expressing biotinylated dCas9 and target-specific or non-targeting sgRNAs. The mRNA expression of β-globin genes and erythroid regulators (GATA1 and KLF1) was analyzed by qRT-PCR. Results are mean±SEM of N=3 independent experiments.

FIGS. 9A to 9E show CAPTURE-Proteomics Identify CRE-Associated Protein Complexes at the β-Globin Cluster, related to FIG. 3. (FIG. 9A) Schematic of iTRAQ-based CAPTURE-Proteomics. Samples prepared from cells expressing target-specific sgRNAs or sgGal4 were isolated by dCas9 affinity purification, followed by in-solution trypsin digestion. The resulting peptides were purified and labeled by multiplexed isobaric tags. The iTRAQ-labeled peptides were mixed, and subjected to multi-dimensional separation and high-resolution MS analysis for peptide identification and quantification. (FIG. 9B) Identification of the high-confidence non-specific proteins in CAPTURE-Proteomics. Non-specific proteins were identified by streptavidin purification followed by iTRAQ-based proteomic analyses from K562 cells expressing BirA-only (Control1), BirA with dCas9 alone (Control2), BirA with dCas9 and sgGal4 (Control3), and BirA with dCas9 and 8 individual β-globin CRE-targeting sgRNAs in which the β-globin cluster was deleted (Control4, BirA-dCas9-sgAll-Globin-KO). The non-specific proteins from each experiment were defined as the proteins with iTRAQ ion intensity ≥100 in at least 2 of 3 replicate experiments. Venn diagrams show the overlap of the non-specific proteins identified from two or four samples. The ‘high-confidence non-specific proteins’ were defined as the proteins identified from all four control samples. (FIG. 9C) The distribution of the high-confidence non-specific proteins in all CAPTURE-Proteomics experiments across iTRAQ ratios (x-axis, top) or P values (x-axis, bottom) is shown. Blue bars represent the percentage (%) of non-specific proteins (left y-axis) in each category. Boxplots represent of the cumulative % of non-specific proteins (right y-axis). Boxes show mean of the data and quartiles. Whiskers show the minimum and maximum of the data. (FIG. 9D) Schematic of data processing, quantification, and identification of locus-specific proteome. The numbers of the significantly enriched locus-specific proteins for each captured region are shown. A diagram of the β-globin cluster showing the positions of sgRNAs used for CAPTURE-Proteomics is shown on the top. (FIG. 9E) CAPTURE-Proteomics identified β-globin CRE-associated proteins. Volcano plots are shown for the CAPTURE-Proteomics in sgHS1, sgHS3 or sgHS4 versus sgGal4-expressing cells. Relative protein levels in the target-specific sgRNA versus sgGal4 samples are plotted on the x-axis as mean log 2 iTRAQ ratios across N replicate experiments. Negative log 10 transformed P values are plotted on the y-axis. Significantly enriched proteins (P≤0.05; iTRAQ ratio ≥1.5) are denoted by black dots, all others by grey dots. Dotted lines indicate 1.5-fold ratio (x-axis) and P value of 0.05 (y-axis). Representative locus-specific chromatin-regulating proteins are denoted by red arrowheads. Representative proteins with iTRAQ ratio ≥1.5 and P >0.05 are denoted by blue arrowheads.

FIGS. 10A to 10H show CAPTURE-Proteomics Identify Candidate Regulators for β-Globin CREs, related to FIG. 3. (FIG. 10A, FIG. 10B) Connectivity network of promoter- or enhancer-associated proteins converged by β-globin CREs. The connectivity was built using interactions (grey lines) between the identified promoter- or enhancer-associated proteins and β-globin CREs. The promoter- or enhancer-associated proteins were defined as the proteins identified to be significantly enriched at any of the captured β-globin promoters (HBG and HBB) or LCR enhancers (HS1-HS4), respectively. Colored nodes denote proteins significantly enriched at single or multiple CREs. Size of the circles denotes the frequency of interactions. Inset tables show the lists of representative proteins associated with β-globin promoters (red), enhancers (blue) or both (green). (FIG. 10C) The chromatin occupancy of BRD4 was validated by ChIP-seq. BRD4 and RNAPII ChIP-seq was performed in K562 cells treated with DMSO or 1 μM of JQ1 for 2 or 6 hours, respectively. (FIG. 10D) JQ1 treatment led to significant downregulation of β-globin genes but not GATA1 or KLF1 in human primary erythroid cells. Results are mean±SEM of three experiments and analyzed by a two-tailed t-test. *P<0.05, **P<0.01, n.s. not significant. (FIG. 10E) Erythroid maturation was assessed using the cell surface markers CD71 and CD235a. (FIG. 10F) Example cytospin of DMSO or JQ1-treated erythroid cells. Scale bars, 20 m. (FIG. 10G) Validation of RNAi knockdown by qRT-PCR. Results are mean±SEM of 1 to 5 shRNAs for each gene in 2 or 3 experiments, and analyzed by a two-sided t-test. (FIG. 10H) Validation of RNAi knockdown of the indicated proteins by Western blot analysis in K562 cells.

FIGS. 11A to 11C show data Analysis Pipelines for CAPTURE-3C-seq, related to FIG. 5. (FIG. 11A) Data preprocessing pipeline for CAPTURE-3C-seq is shown. The output data files and the processing steps are shown as blue and red boxes, respectively. (FIG. 11B) Statistical analysis pipeline for CAPTURE-3C-seq is shown. (FIG. 11C) The comaprison between CAPTURE-ChIP-seq, ChIA-PET (RNAPII and CTCF), UMI-4C, DNase Hi-C (genome-wide or LCR-targeted) and in situ Hi-C is shown. Compared with RNAPII and CTCF ChIA-PET data in K562 cells (Consortium, 2012; Li et al., 2012), CAPTURE-3C-seq shows significantly higher % of unique PETs and on-target enrichment as measured by the number of PET interactions per kilobases of bait region per million mapped reads. Compared with Hi-C data in K562 cells (Ma et al., 2015; Rao et al., 2014), CAPTURE-3C-seq shows comparable or slightly higher % of unique PETs but significantly higher on-target enrichment. Compared to UMI-4C (Schwartzman et al., 2016), CAPTURE-3C-seq displayed higher % of unique PETs but comparable or slightly lower on-target enrichment. The unique PETs were defined as pair-end sequence tags with distinct genomic locations at one or both sides of the pair-end reads.

FIGS. 12A and 12B show CAPTURE-3C-seq of Locus-Specific DNA Interactions by Multiple sgRNAs, related to FIG. 5. (FIG. 12A) Schematic of CAPTURE-3C-seq analysis of HS2 or HS3-mediated long-range DNA interactions by four independent sgRNAs at various positions of the captured region. The distance between sgRNAs and the DpnII sites is shown. (FIG. 12B) Browser view of the long-range DNA interactions at HS2 or HS3 captured by four independent sgRNAs. Contact profiles compiled from two or three CAPTURE-3C-seq experiments for each sgRNA including the density map and interactions (or loops) are shown. The statistical significance of interactions was determined by the Bayes factor (BF), and is indicated by the darkness of each interaction loop according to the color scale bars. Interactions with BF ≥20 were considered high-confidence long-range DNA interactions. The DHS, ChIP-seq (H3K27ac, H3K4me1, H3K4me3, CTCF, and RNAPII), RNA-seq, and ChromHMM data are shown for comparison. The locations of the LCR (HS1 to HS5) and the 3′HS1 insulator are shown as shaded lines. The TSS for β-globin genes are shown as dashed line.

FIG. 13. CAPTURE-3C-seq of Locus-Specific DNA Interactions at Multiple β-Globin CREs, Related to FIG. 5. Browser view of the long-range DNA interaction profiles at dCas9-captured β-globin CREs is shown (chr11:5,222,500-5,323,700; hg19). Contact profiles compiled from two or three CAPTURE-3C-seq experiments including the density map and interactions (or loops) are shown. ChIA-PET (Consortium, 2012; Li et al., 2012), UMI-4C (Schwartzman et al., 2016), 5C (Naumova et al., 2013), DNase Hi-C (Ma et al., 2015), in situ Hi-C(Rao et al., 2014), DHS, ChIP-seq, RNA-seq, and ChromHMM data are shown for comparison.

FIGS. 14A to 14C shows a CAPTURE-3C-seq of Locus-Specific DNA Interactions at HS3 and HBD-1kb, related to FIGS. 5 and 6. (FIG. 14A) A zoom-out browser view of the long-range DNA interactions at HS3 (chr11:5,214,997-5,449,997; hg19) is shown. Contact profiles compiled from 3 experiments including the density map, interactions (or loops) and pair-end tags (PETs), along with the ChIA-PET, 5C, Hi-C, DHS, ChIP-seq, RNA-seq, and ChromHMM data are shown for comparison. (FIG. 14B) Browser view of the long-range DNA interactions at the HBD-1kb, HBD-1.5kb and HBD-2kb regions (chr11:5,222,500-5,323,700; hg19) is shown. Schematic of the 3.5 kb cis-element along with the deletions mapped in prior studies are shown on the top. A 3.5 kb putative cis-element (chr11:5,255,859-5,259,368; hg19) was defined by the upstream breakpoint of the HPFH-1 deletion and the TSS of HBD. The sgRNAs (HBD-1kb, HBD-1.5kb and HBD-2kb) used for CAPTURE-3C-seq and CAPTURE-Proteomics are indicated by arrowheads. (FIG. 14C) CAPTURE-Proteomics identified HBD-1.5kb and HBD-2kb-associated proteins. Volcano plots are shown for the iTRAQ-based proteomics of affinity purification in sgHBD-1.5kb or sgHBD-2kb versus sgGal4-expressing cells (N=3 replicate experiments).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present inventors developed a developed a CRISPR affinity purification in situ of regulatory elements (CAPTURE) approach to unbiasedly identify locus-specific chromatin-regulating protein, RNA complexes and long-range DNA interactions. Using an in vivo biotinylated nuclease-deficient Cas9 protein and sequence-specific guide RNAs, the inventors show high-resolution and selective isolation of chromatin interactions at a single copy genomic locus. Purification of human telomeres using CAPTURE identifies known and new telomeric factors. In situ capture of individual constituents of the enhancer cluster controlling human β-globin genes establishes evidence for composition-based hierarchical organization. Furthermore, unbiased analysis of chromatin interactions at disease-associated cis-elements and developmentally regulated super-enhancers reveals spatial features causally control gene transcription. Thus, the present invention allows for comprehensive and unbiased analysis of locus-specific regulatory composition provides mechanistic insight into genome structure and function in development and disease.

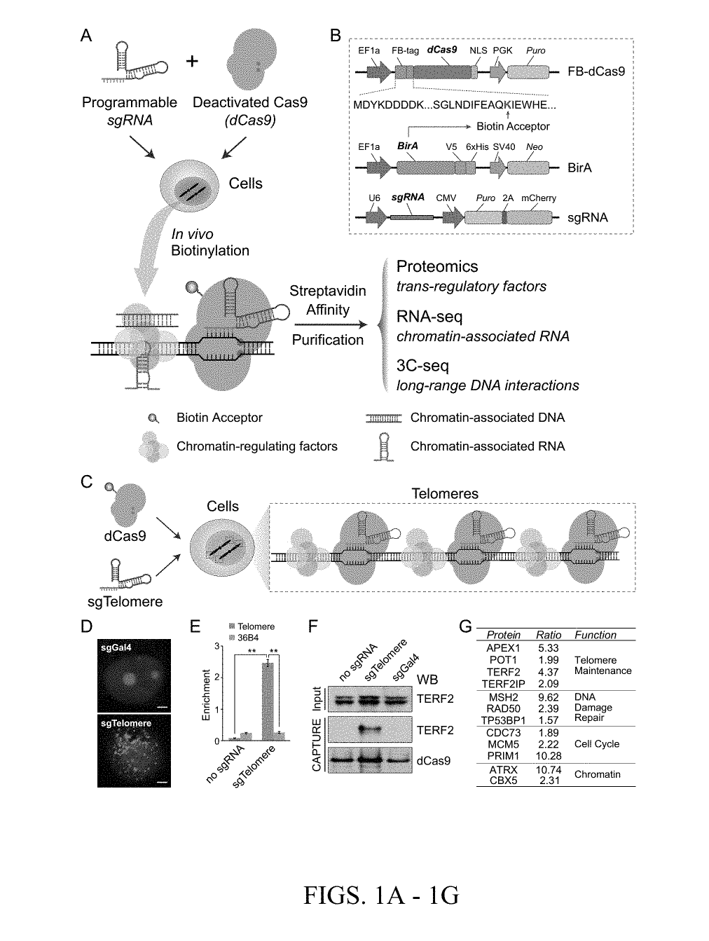

In Situ Capture of Chromatin Interactions by dCas9-Mediated Affinity Purification. To facilitate the analysis of native CREs, the inventors developed a method to isolate chromatin interactions in situ (FIG. 1A). The core components of CRISPR include Cas9 and a single guide RNA (sgRNA), which serves to direct Cas9 to a target genomic sequence (Cong et al., 2013; Mali et al., 2013). The inventors engineered an N-terminal FLAG and biotin-acceptor-site (FB)-tagged deactivated Cas9 (dCas9) (FIG. 1B). Upon in vivo biotinylation of dCas9 by the biotin ligase BirA together with sequence-specific sgRNAs in mammalian cells, the genomic locus-associated macromolecules are isolated by high affinity streptavidin purification. The purified protein, RNA and DNA complexes are identified and analyzed by mass spectrometry (MS)-based proteomics and high-throughput sequencing for study of native CRE-regulating proteins, RNA, and long-range DNA interactions, respectively (FIG. 1A).

This approach has several advantages including: 1) high sensitivity—the affinity between biotin and streptavidin with K_{d}=10⁻¹⁴ mol/L is >1000-fold higher than antibody-mediated interactions (Kim et al., 2009a; Schatz, 1993), thus allowing for more efficient and stable capture of protein-DNA complexes. 2) High specificity—this approach avoids using antibodies which significantly reduces non-specific binding. In addition, the extraordinary stability of biotin-streptavidin allows for stringent purification to eliminate protein contamination. 3) Adaptability for multiplexed approaches—the dCas9/sgRNA system can be manipulated by altering sgRNA sequences or combinations, thus allowing for medium- to high-throughput analysis of chromatin interactions. Taken together, this new approach, which the inventors named CAPTURE (CRISPR Affinity Purification in situ of Regulatory Elements), has the potential to expedite the analysis of chromatin-templated events by characterizing the entire set of interacting macromolecules and how composition changes during cellular differentiation.

In Situ CAPTURE of Human Telomeres. As a proof-of-principle, the inventors used CAPTURE to isolate human telomeres in K562 cells (FIG. 1C). The inventors employed a validated telomere-targeting sgRNA (sgTelomere; FIG. 1C) (Chen et al., 2013), which displayed specific labeling of telomeres by the dCas9-EGFP fusion protein, in contrast to the diffuse nucleolar localization of the non-targeting dCas9-EGFP (FIG. 1D). Upon stable co-expression of sgTelomere and biotinylated dCas9, the inventors observed significant enrichment of telomeric DNA (FIG. 1E). The known telomere-associated protein TERF2 was highly enriched in sgTelomere-expressing but not control samples expressing dCas9 alone (no sgRNA) or the non-targeting sgGal4 (FIG. 1F). Most importantly, by iTRAQ-based proteomics, the inventors identified many known telomere maintenance proteins (Dejardin and Kingston, 2009; Lewis and Wuttke, 2012) and new telomere-associated proteins (FIG. 1G and Table 3).

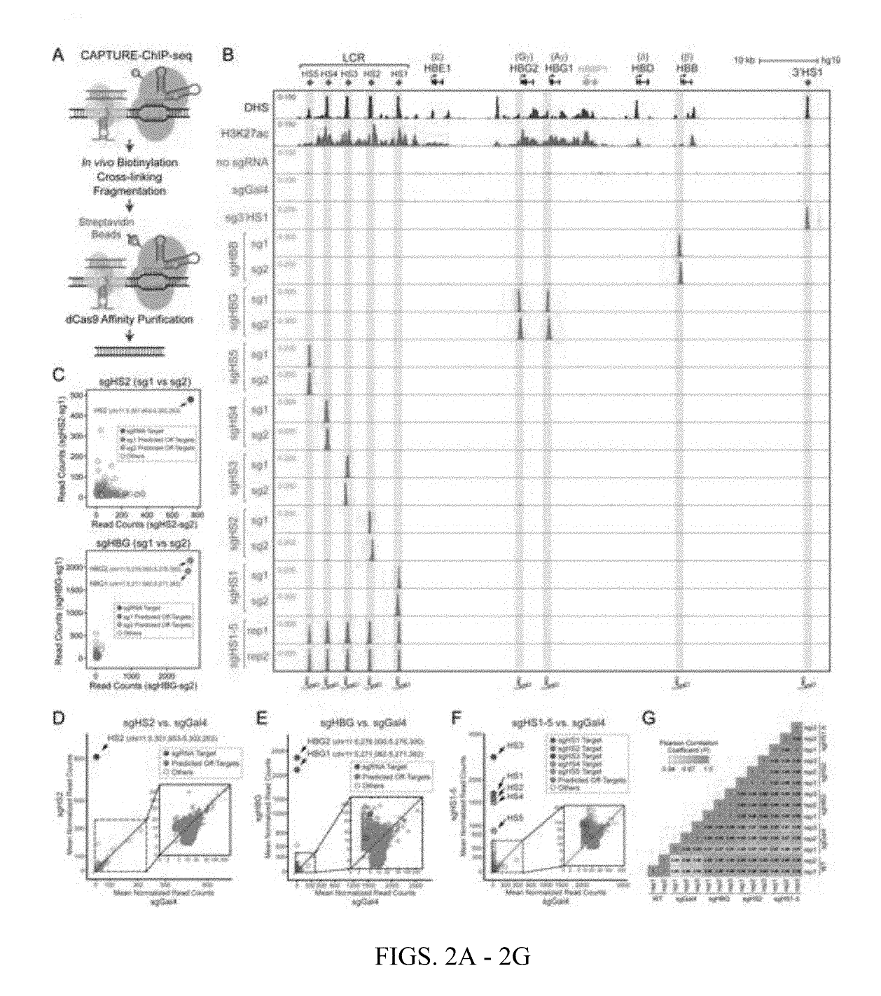

In Situ CAPTURE of β-Globin Cluster. To validate the CAPTURE approach for identifying single copy CREs, the inventors focused on the human β-globin cluster containing five β-like globin genes controlled by a shared enhancer cluster (locus control region or LCR) with five discrete DHS (HS1 to HS5). The inventors designed two or three independent sgRNAs for each promoter (HBG1, HBG2 and HBB), enhancer (HS1 to HS4) or insulator (HS5) (Tables 1 and 2). Upon co-expression of sgRNAs and dCas9, K562 chromatin was cross-linked and purified, followed by sequencing of the captured DNA (‘CAPTURE-ChIP-seq’; FIG. 2A). The inventors observed specific and significant enrichment of discrete sgRNA-targeted regions (FIG. 2B). For example, expression of two sgRNAs for HS1 (sgHS1-sg1 and sg2) led to significant enrichment of HS1 but no other enhancers. Because the sequence similarity between HBG1 and HBG2, the sgRNAs targeting HBG promoters (sgHBG-sg1 and sg2) do not distinguish the two genes. Consistently, co-expression of sgHBG and dCas9 resulted in significant enrichment of both HBG genes. In contrast, binding of dCas9 to β-globin cluster was undetectable when expressed alone (no sgRNA) or with the non-targeting sgGal4. Importantly, co-expression of five sgRNAs (sgHS1-5) led to simultaneous capture of all five LCR enhancers, demonstrating that the CAPTURE system can be adapted for multiplexed analysis of independent CREs. Furthermore, by comparing ChIP-seq intensity using two or three independent sgRNAs, the inventors observed highly specific enrichment of each captured region with minimal off-targets (FIG. 2C, 8D). Given the consistent performance, hereafter the inventors focus on one sgRNA (sg1, Table 2) for each region unless otherwise specified.

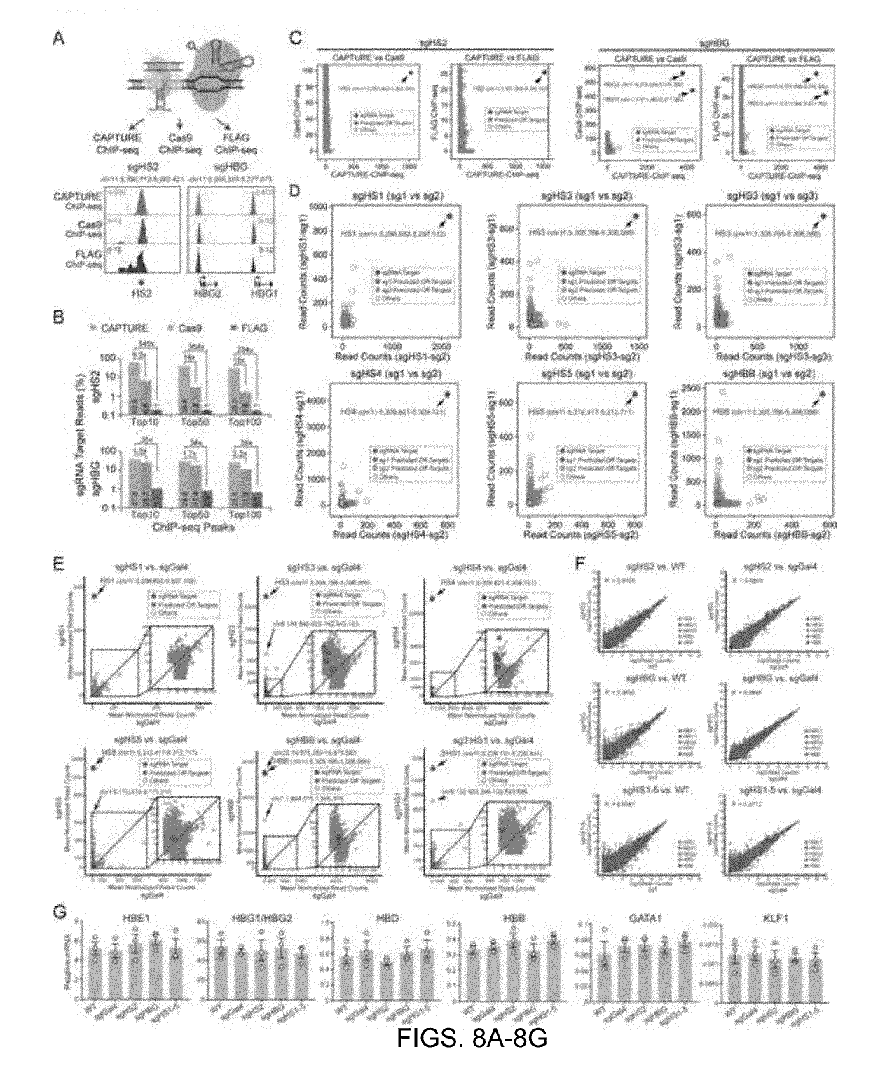

Genome-Wide Enrichment and Specificity of CAPTURE. To identify locus-specific interactions, it is critical to evaluate the on-target enrichment and off-target effects. The inventors first compared CAPTURE-ChIP-seq with dCas9 or FLAG antibody-based ChIP-seq using sgHS2 and sgHBG, and observed significantly higher binding intensity by CAPTURE-ChIP-seq (FIG. 8A; Table 1). Among the top 100 peaks by sgHS2, CAPTURE-ChIP-seq led to 18- or 284-fold on-target enrichment compared to dCas9 or FLAG-based ChIP-seq, respectively (FIG. 8B). At the global scale, CAPTURE-ChIP-seq resulted in highly specific enrichment of HS2 or HBG with many fewer off-targets than antibody-based ChIP-seq (FIG. 8C). These results provide evidence that the CAPTURE approach allows for more efficient purification of targeted chromatin through improved on-target enrichment and elimination of potential off-targets.

The inventors next assessed the genome-wide specificity by comparing dCas9 binding in cells expressing target-specific sgRNAs or sgGal4. Specifically, recruitment of dCas9 by sgHS2 resulted in highly specific enrichment of HS2 with no additional significant dCas9 binding (FIG. 2D). Similarly, recruitment of dCas9 by sgHBG led to specific enrichment of HBG1 and HBG2, whereas none of the predicted off-targets were significantly enriched (FIG. 2E). Moreover, multiplexed capture by sgHS1-5 resulted in identification of LCR enhancers as the top enriched binding sites (FIG. 2F). Similar results were obtained with 12 other sgRNAs (FIGS. 8D, 8E; Table 1). RNA-seq in target-specific sgRNAs, sgGal4 and wild-type (WT) K562 cells revealed minimal transcriptomic changes (FIG. 2G; 8F). The expression of β-globin mRNAs remained unchanged (FIG. 8G), suggesting that the dCas9 capture did not interfere with the expression of endogenous genes. Together, these analyses establish that the CAPTURE system is highly specific to target loci and can be used to isolate locus-specific regulatory components.

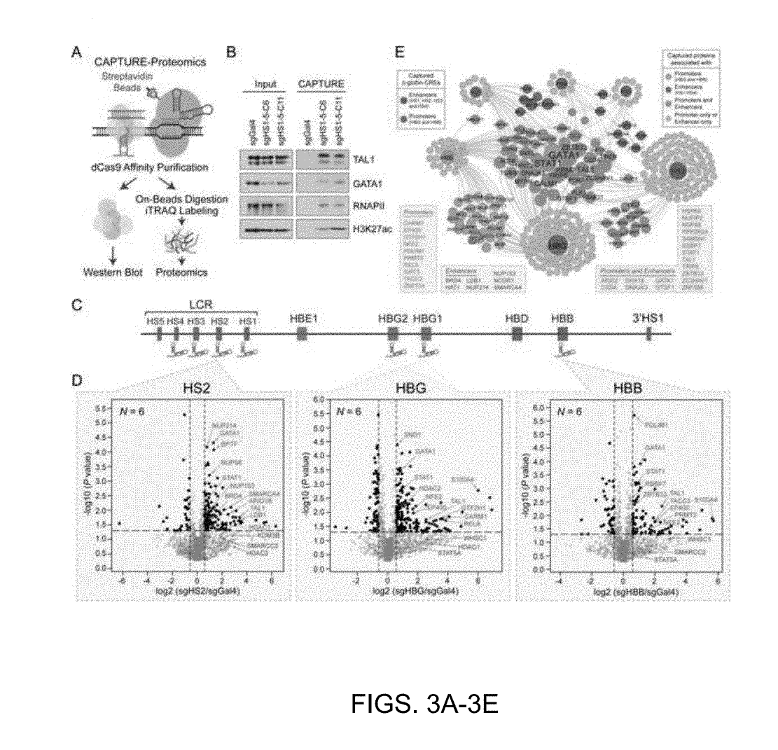

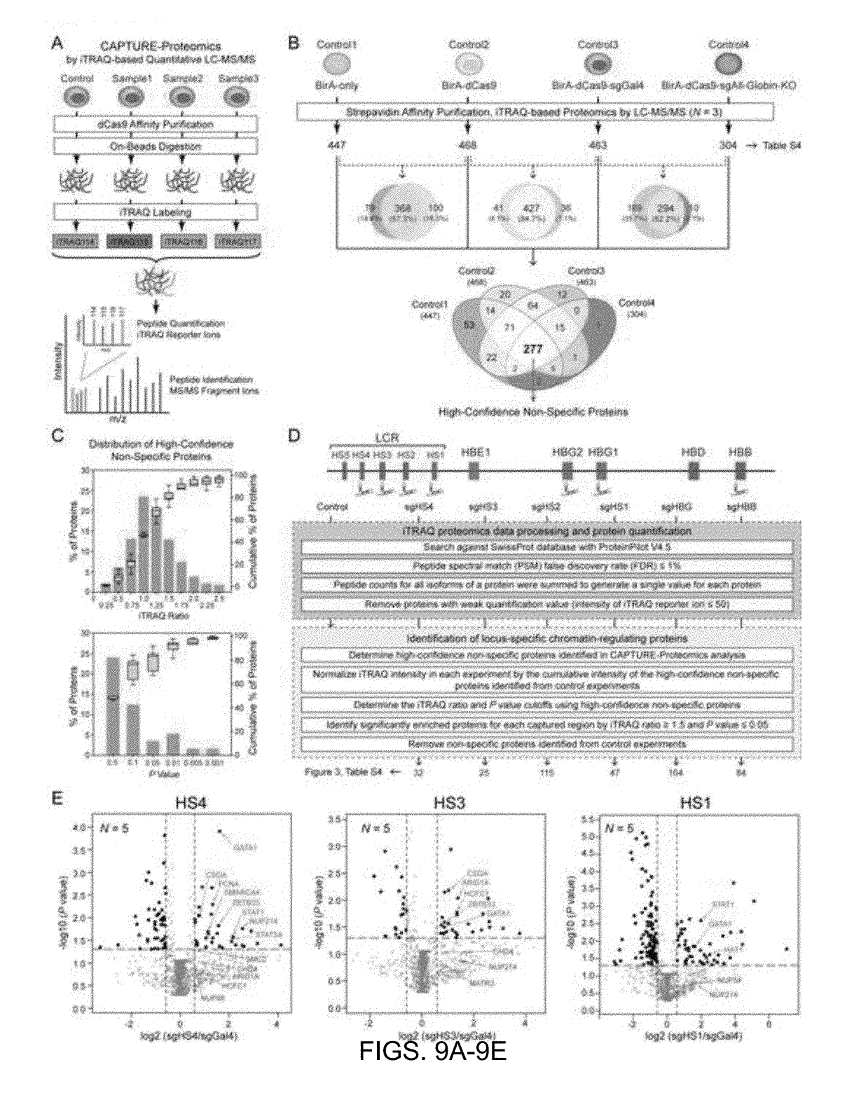

CAPTURE-Proteomics Identify Trans-Acting Regulators of β-Globin Genes. A major challenge for proteomic analysis of a single genomic locus is the need for a sufficient amount of purified proteins. Hence, the inventors optimized several components of the procedures including protein purification, peptide isolation, quantitative proteomic profiling, and developed the ‘CAPTURE-Proteomics’ approach to identify locus-specific protein complexes (FIG. 3A; 9A). The inventors first performed purification in control cell lines to categorize the endogenous biotinylated proteins and/or dCas9-associated non-specific proteins (FIG. 9B). Specifically, the inventors identified proteins purified from K562 cells expressing BirA-only, BirA with dCas9, BirA with dCas9 and sgGal4, and BirA with dCas9 and β-globin CRE-specific sgRNAs in which the endogenous β-globin cluster was deleted (BirA-dCas9-sgAll-Globin-KO; Method Details). Compiled from three experiments, the inventors identified 304 to 468 proteins from individual controls, including 277 ‘high-confidence non-specific proteins’ present in all controls (FIG. 9B).

The inventors next determined whether known β-globin regulators can be isolated. Co-expression of dCas9 with sgHS1-5 led to significant enrichment of the erythroid TFs (GATA1 and TAL1) required for globin enhancers, together with RNA polymerase II (RNAPII) and acetylated H3K27 (H3K27ac) (FIG. 3B). The inventors then performed iTRAQ-based quantitative proteomics of captured β-globin CREs (FIG. 3C). Relative protein abundance associated with the captured CRE versus sgGal4 was determined by the ratio of the iTRAQ reporter ion intensity. The significance of enrichment (P value) for each protein was calculated by paired t-test of the log₂ iTRAQ ratios in replicate experiments. The inventors surveyed the distribution of ‘high-confidence non-specific proteins’ in all experiments, and observed that 78.3% and 79.8% of them had iTRAQ ratio <1.5 and P value >0.05 (FIG. 9C). Therefore, the inventors employed the iTRAQ ratio ≥1.5 and P value ≤0.05 as the cutoffs and identified 25 to 164 candidate locus-specific proteins (FIGS. 3D, 9D, 9E).

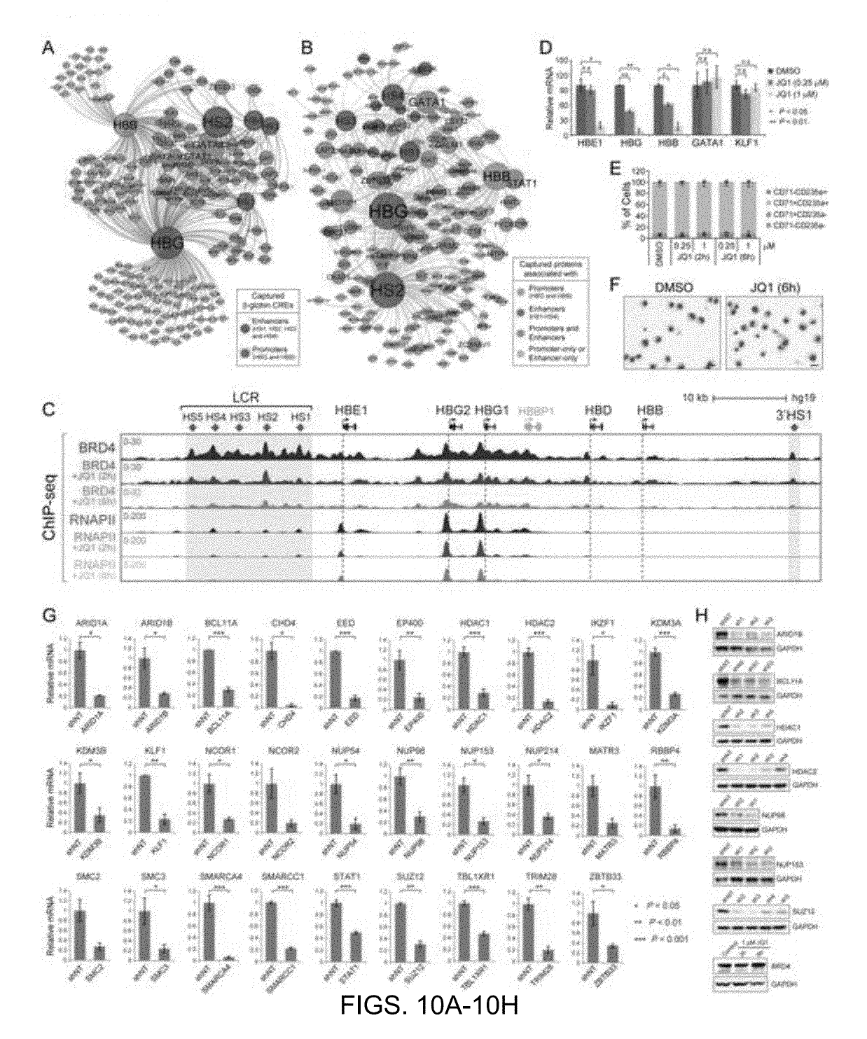

Using CAPTURE-Proteomics, the inventors identified many known factors including GATA1, TAL1, NFE2, components of the SWI/SNF (ARIDIA, ARID1B, SMARCA4 and SMARCC1) and NuRD (CHD4, RBBP4, RBBP7, HDAC1 and HDAC2) complexes (Kim et al., 2009b; Miccio and Blobel, 2010; Xu et al., 2013) at β-globin CREs. More importantly, by locus-specific proteomics, the inventors identified new β-globin CRE-associated complexes including the nucleoporins (NUP98, NUP153 and NUP214), components of the large multiprotein nuclear pore complexes (NPCs), at LCR enhancers (FIGS. 3D, 3E). In addition, BRD4 and LDB1 were identified at LCR enhancers, whereas the NuA4 acetyltransferase (EP400) and transcriptional initiation complex (GTF2H1) were found at 3-globin promoters. Furthermore, the inventors observed that the HBG and HBB promoters shared many interacting proteins and clustered closely in protein-DNA connectivity networks (FIGS. 3E, 10A, 10B). By contrast, the distal enhancers (HS1, HS3 and HS4) clustered together to form a distinct subdomain through enhancer-associated proteins, whereas HS2 shared interacting proteins with both subdomains. These analyses provide initial evidence for the composition-based hierarchical organization of the β-globin CREs.

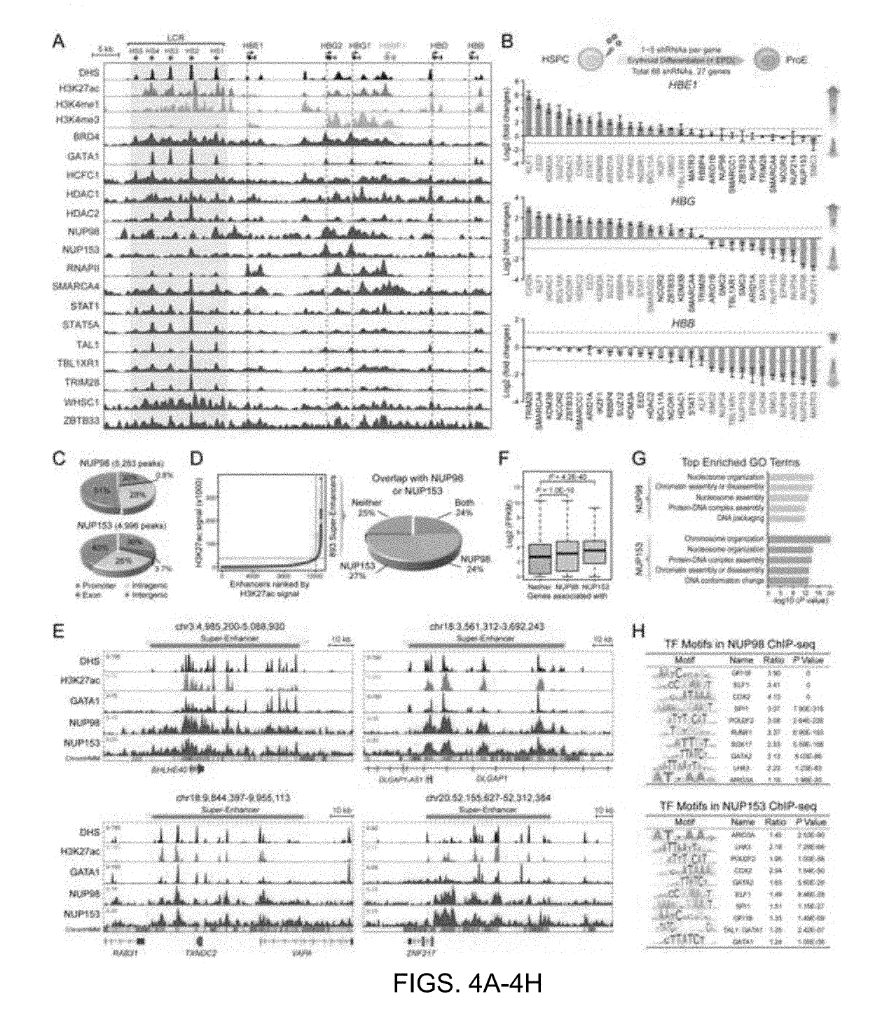

Identification of New Regulators of β-Globin Genes and Erythroid Enhancers. The inventors validated the binding of a subset of the identified proteins in K562 cells by ChIP-seq (FIG. 4A; Table 1). Importantly, among the factors not previously implicated in β-globin regulation, the inventors confirmed the nucleoporins (NUP98 and NUP153), STAT proteins (STAT1 and STAT5A), TBL1XR1, HCFC1, TRIM28/KAP1, WHSC1/NSD2, and ZBTB33/KAISO to be significantly enriched at one or multiple LCR enhancers by CAPTURE-Proteomics and ChIP-seq. To establish the functional roles, the inventors performed RNAi-mediated loss-of-function analysis in human primary erythroid cells (FIGS. 4B, 10G, 10H; Table 2). Specifically, depletion of 17 of 27 factors led to significant upregulation or downregulation of HBG (≥2-fold; FIG. 4B). Similarly, depletion of 15 or 11 of 27 factors led to significant changes in HBB or HBE1 (≥2-fold), respectively. Notably, depletion of NUP98, NUP153 and NUP214 led to marked downregulation of HBG (2.8 to 7.3-fold) and HBB (3.3 to 5.6-fold), suggesting that the NUP proteins are directly or indirectly required for the activation of β-globin genes.

TABLE 1
List of Genomic Datasets,
Related to STAR Methods.

| | | | GEO Accession | |
|---|---|---|---|---|
| Datasets | Data Type | Cell Type | Number | Citation |
| CAPTURE-ChIP- | CAPTURE- | K562 FB-dCas9/BirA clone6 | GSM2635188 | This study |
| seq_K562_sgHS1-rep1 | ChIP-seq | | | |
| CAPTURE-ChIP- | CAPTURE- | K562 FB-dCas9/BirA clone11 | GSM2635189 | This study |
| seq_K562_sgHS1-rep2 | ChIP-seq | | | |
| CAPTURE-ChIP- | CAPTURE- | K562 FB-dCas9/BirA clone6 | GSM2635190 | This study |
| seq_K562_sgHS2-rep1 | ChIP-seq | | | |
| CAPTURE-ChIP- | CAPTURE- | K562 FB-dCas9/BirA clone6 | GSM2635191 | This study |
| seq_K562_sgHS2-rep2 | ChIP-seq | | | |
| CAPTURE-ChIP- | CAPTURE- | K562 FB-dCas9/BirA clone6 | GSM2635192 | This study |
| seq_K562_sgHS2-rep3 | ChIP-seq | | | |
| CAPTURE-ChIP- | CAPTURE- | K562 FB-dCas9/BirA clone11 | GSM2635193 | This study |
| seq_K562_sgHS2-rep4 | ChIP-seq | | | |
| CAPTURE-ChIP- | CAPTURE- | K562 FB-dCas9/BirA clone11 | GSM2635194 | This study |
| seq_K562_sgHS2-rep5 | ChIP-seq | | | |
| CAPTURE-ChIP- | CAPTURE- | K562 FB-dCas9/BirA clone6 | GSM2635195 | This study |
| seq_K562_sgHS3-rep1 | ChIP-seq | | | |
| CAPTURE-ChIP- | CAPTURE- | K562 FB-dCas9/BirA clone6 | GSM2635196 | This study |
| seq_K562_sgHS3-rep2 | ChIP-seq | | | |
| CAPTURE-ChIP- | CAPTURE- | K562 FB-dCas9/BirA clone6 | GSM2635197 | This study |
| seq_K562_sgHS3-rep3 | ChIP-seq | | | |
| CAPTURE-ChIP- | CAPTURE- | K562 FB-dCas9/BirA clone6 | GSM2635198 | This study |
| seq_K562_sgHS4-rep1 | ChIP-seq | | | |
| CAPTURE-ChIP- | CAPTURE- | K562 FB-dCas9/BirA clone6 | GSM2635199 | This study |
| seq_K562_sgHS4-rep2 | ChIP-seq | | | |
| CAPTURE-ChIP- | CAPTURE- | K562 FB-dCas9/BirA clone6 | GSM2635200 | This study |
| seq_K562_sgHS5-rep1 | ChIP-seq | | | |
| CAPTURE-ChIP- | CAPTURE- | K562 FB-dCas9/BirA clone11 | GSM2635201 | This study |
| seq_K562_sgHS5-rep2 | ChIP-seq | | | |
| CAPTURE-ChIP- | CAPTURE- | K562 FB-dCas9/BirA clone11 | GSM2635202 | This study |
| seq_K562_sgHS5-rep3 | ChIP-seq | | | |
| CAPTURE-ChIP- | CAPTURE- | K562 FB-dCas9/BirA clone6 | GSM2635203 | This study |
| seq_K562_sgHBB-rep1 | ChIP-seq | | | |
| CAPTURE-ChIP- | CAPTURE- | K562 FB-dCas9/BirA clone6 | GSM2635204 | This study |
| seq_K562_sgHBG-rep1 | ChIP-seq | | | |
| CAPTURE-ChIP- | CAPTURE- | K562 FB-dCas9/BirA clone6 | GSM2635205 | This study |
| seq_K562_sgHBG-rep2 | ChIP-seq | | | |
| CAPTURE-ChIP- | CAPTURE- | K562 FB-dCas9/BirA clone11 | GSM2635206 | This study |
| seq_K562_sgHBG-rep3 | ChIP-seq | | | |
| CAPTURE-ChIP- | CAPTURE- | K562 FB-dCas9/BirA clone11 | GSM2635207 | This study |
| seq_K562_sgHBG-rep4 | ChIP-seq | | | |
| CAPTURE-ChIP- | CAPTURE- | K562 FB-dCas9/BirA clone6 | GSM2635208 | This study |
| seq_K562_sgHBD-1kb- | ChIP-seq | | | |
| rep1 | | | | |
| CAPTURE-ChIP- | CAPTURE- | K562 FB-dCas9/BirA clone6 | GSM2635209 | This study |
| seq_K562_sgHBD-1kb- | ChIP-seq | | | |
| rep2 | | | | |
| CAPTURE-ChIP- | CAPTURE- | K562 FB-dCas9/BirA clone6 | GSM2635210 | This study |
| seq_K562_sg3′HS1- | ChIP-seq | | | |
| rep1 | | | | |
| CAPTURE-ChIP- | CAPTURE- | K562 FB-dCas9/BirA clone6 | GSM2635211 | This study |
| seq_K562_sg3′HS1- | ChIP-seq | | | |
| rep2 | | | | |
| CAPTURE-ChIP- | CAPTURE- | K562 FB-dCas9/BirA clone6 | GSM2635212 | This study |
| seq_K562_sgHS1-5- | ChIP-seq | | | |
| rep1 | | | | |
| CAPTURE-ChIP- | CAPTURE- | K562 FB-dCas9/BirA clone6 | GSM2635213 | This study |
| seq_K562_sgHS1-5- | ChIP-seq | | | |
| rep2 | | | | |
| CAPTURE-ChIP- | CAPTURE- | K562 FB-dCas9/BirA clone6 | GSM2635214 | This study |
| seq_K562_sgHS1-5- | ChIP-seq | | | |
| rep3 | | | | |
| CAPTURE-ChIP- | CAPTURE- | K562 FB-dCas9/BirA clone6 | GSM2635215 | This study |
| seq_K562_sgHS1-5- | ChIP-seq | | | |
| rep4 | | | | |
| CAPTURE-ChIP- | CAPTURE- | K562 FB-dCas9/BirA clone6 | GSM2635216 | This study |
| seq_K562_sgHS1-5- | ChIP-seq | | | |
| rep5 | | | | |
| CAPTURE-ChIP- | CAPTURE- | K562 FB-dCas9/BirA clone6 | GSM2635217 | This study |
| seq_K562_sgHS1-5- | ChIP-seq | | | |
| rep6 | | | | |
| CAPTURE-ChIP- | CAPTURE- | K562 FB-dCas9/BirA clone6 | GSM2635218 | This study |
| seq_K562_sgGal4-rep1 | ChIP-seq | | | |
| CAPTURE-ChIP- | CAPTURE- | K562 FB-dCas9/BirA clone6 | GSM2635219 | This study |
| seq_K562_sgGal4-rep2 | ChIP-seq | | | |
| CAPTURE-ChIP- | CAPTURE- | K562 FB-dCas9/BirA clone6 | GSM2635220 | This study |
| seq_K562_sgGal4-rep3 | ChIP-seq | | | |
| CAPTURE-ChIP- | CAPTURE- | K562 FB-dCas9/BirA clone6 | GSM2635221 | This study |
| seq_K562_sgGal4-rep4 | ChIP-seq | | | |
| CAPTURE-ChIP- | CAPTURE- | K562 FB-dCas9/BirA clone6 | GSM2635222 | This study |
| seq_K562_no_sgRNA- | ChIP-seq | | | |
| rep1 | | | | |
| CAPTURE-ChIP- | CAPTURE- | K562 FB-dCas9/BirA clone6 | GSM2635223 | This study |
| seq_K562_sgHS1-sg2- | ChIP-seq | | | |
| rep1 | | | | |
| CAPTURE-ChIP- | CAPTURE- | K562 FB-dCas9/BirA clone6 | GSM2635224 | This study |
| seq_K562_sgHS1-sg2- | ChIP-seq | | | |
| rep2 | | | | |
| CAPTURE-ChIP- | CAPTURE- | K562 FB-dCas9/BirA clone6 | GSM2635225 | This study |
| seq_K562_sgHS2-sg2 | ChIP-seq | | | |
| CAPTURE-ChIP- | CAPTURE- | K562 FB-dCas9/BirA clone6 | GSM2635226 | This study |
| seq_K562_sgHS3-sg2 | ChIP-seq | | | |
| CAPTURE-ChIP- | CAPTURE- | K562 FB-dCas9/BirA clone6 | GSM2635227 | This study |
| seq_K562_sgHS4-sg2- | ChIP-seq | | | |
| rep1 | | | | |
| CAPTURE-ChIP- | CAPTURE- | K562 FB-dCas9/BirA clone6 | GSM2635228 | This study |
| seq_K562_sgHS4-sg2- | ChIP-seq | | | |
| rep2 | | | | |
| CAPTURE-ChIP- | CAPTURE- | K562 FB-dCas9/BirA clone6 | GSM2635229 | This study |
| seq_K562_sgHS5-sg2- | ChIP-seq | | | |
| rep1 | | | | |
| CAPTURE-ChIP- | CAPTURE- | K562 FB-dCas9/BirA clone6 | GSM2635230 | This study |
| seq_K562_sgHS5-sg2- | ChIP-seq | | | |
| rep2 | | | | |
| CAPTURE-ChIP- | CAPTURE- | K562 FB-dCas9/BirA clone6 | GSM2635231 | This study |
| seq_K562_sgHBB-sg2- | ChIP-seq | | | |
| rep1 | | | | |
| CAPTURE-ChIP- | CAPTURE- | K562 FB-dCas9/BirA clone6 | GSM2635232 | This study |
| seq_K562_sgHBB-sg2- | ChIP-seq | | | |
| rep2 | | | | |
| CAPTURE-ChIP- | CAPTURE- | K562 FB-dCas9/BirA clone6 | GSM2635233 | This study |
| seq_K562_sgHBG-sg2- | ChIP-seq | | | |
| rep1 | | | | |
| CAPTURE-ChIP- | CAPTURE- | K562 FB-dCas9/BirA clone6 | GSM2635234 | This study |
| seq_K562_sgHBG-sg2- | ChIP-seq | | | |
| rep2 | | | | |
| CAPTURE-ChIP- | CAPTURE- | K562 FB-dCas9/BirA clone6 | GSM2635235 | This study |
| seq_K562_sgHS2- | ChIP-seq | | | |
| Streptavidin | | | | |
| CAPTURE-ChIP- | CAPTURE- | K562 FB-dCas9/BirA clone6 | GSM2635236 | This study |
| seq_K562_sgHS2-Cas9 | ChIP-seq | | | |
| CAPTURE-ChIP- | CAPTURE- | K562 FB-dCas9/BirA clone6 | GSM2635237 | This study |
| seq_K562_sgHS2-Flag | ChIP-seq | | | |
| CAPTURE-ChIP- | CAPTURE- | K562 FB-dCas9/BirA clone6 | GSM2635238 | This study |
| seq_K562_sgHBG- | ChIP-seq | | | |
| Streptavidin | | | | |
| CAPTURE-ChIP- | CAPTURE- | K562 FB-dCas9/BirA clone6 | GSM2635239 | This study |
| seq_K562_sgHBG-Cas9 | ChIP-seq | | | |
| CAPTURE-ChIP- | CAPTURE- | K562 FB-dCas9/BirA clone6 | GSM2635240 | This study |
| seq_K562_sgHBG-Flag | ChIP-seq | | | |
| CAPTURE-ChIP- | CAPTURE- | KH2 FB-dCas9-IRES-BirA | GSM2635241 | This study |
| seq_ESC-KH2_sgEsrrb- | ChIP-seq | clone5 | | |
| SE | | | | |
| CAPTURE-ChIP- | CAPTURE- | KH2 FB-dCas9-IRES-BirA | GSM2635242 | This study |
| seq_ESC-KH2_sgOct4- | ChIP-seq | clone5 | | |
| SE | | | | |
| CAPTURE-ChIP- | CAPTURE- | KH2 FB-dCas9-IRES-BirA | GSM2635243 | This study |
| seq_ESC-KH2_sgSox2- | ChIP-seq | clone5 | | |
| SE | | | | |
| CAPTURE-ChIP- | CAPTURE- | KH2 FB-dCas9-IRES-BirA | GSM2635244 | This study |
| seq_ESC-KH2_sgUtf1- | ChIP-seq | clone5 | | |
| SE | | | | |
| CAPTURE-ChIP- | CAPTURE- | KH2 FB-dCas9-IRES-BirA | GSM2635245 | This study |
| seq_EB-KH2_sgEsrrb- | ChIP-seq | clone5 | | |
| SE | | | | |
| CAPTURE-ChIP- | CAPTURE- | KH2 FB-dCas9-IRES-BirA | GSM2635246 | This study |
| seq_EB-KH2_sgOct4- | ChIP-seq | clone5 | | |
| SE | | | | |
| CAPTURE-ChIP- | CAPTURE- | KH2 FB-dCas9-IRES-BirA | GSM2635247 | This study |
| seq_EB-KH2_sgSox2- | ChIP-seq | clone5 | | |
| SE | | | | |
| CAPTURE-ChIP- | CAPTURE- | KH2 FB-dCas9-IRES-BirA | GSM2635248 | This study |
| seq_EB-KH2_sgUtf1- | ChIP-seq | clone5 | | |
| SE | | | | |
| CAPTURE-3C- | CAPTURE- | K562 FB-dCas9/BirA clone6 | GSM2635065 | This study |
| seq_K562_sgHS1-rep1 | 3C-seq | | | |
| CAPTURE-3C- | CAPTURE- | K562 FB-dCas9/BirA clone6 | GSM2635066 | This study |
| seq_K562_sgHS1-rep2 | 3C-seq | | | |
| CAPTURE-3C- | CAPTURE- | K562 FB-dCas9/BirA clone6 | GSM2635067 | This study |
| seq_K562_sgHS1- | 3C-seq | | | |
| combined | | | | |
| CAPTURE-3C- | CAPTURE- | K562 FB-dCas9/BirA clone6 | GSM2635068 | This study |
| seq_K562_sgHS2-rep1 | 3C-seq | | | |
| CAPTURE-3C- | CAPTURE- | K562 FB-dCas9/BirA clone6 | GSM2635069 | This study |
| seq_K562_sgHS2-rep2 | 3C-seq | | | |
| CAPTURE-3C- | CAPTURE- | K562 FB-dCas9/BirA clone6 | GSM2635070 | This study |
| seq_K562_sgHS2-rep3 | 3C-seq | | | |
| CAPTURE-3C- | CAPTURE- | K562 FB-dCas9/BirA clone6 | GSM2635071 | This study |
| seq_K562_sgHS2- | 3C-seq | | | |
| combined | | | | |
| CAPTURE-3C- | CAPTURE- | K562 FB-dCas9/BirA clone6 | GSM2635072 | This study |
| seq_K562_sgHS3-rep1 | 3C-seq | | | |
| CAPTURE-3C- | CAPTURE- | K562 FB-dCas9/BirA clone6 | GSM2635073 | This study |
| seq_K562_sgHS3-rep2 | 3C-seq | | | |
| CAPTURE-3C- | CAPTURE- | K562 FB-dCas9/BirA clone6 | GSM2635074 | This study |
| seq_K562_sgHS3-rep3 | 3C-seq | | | |
| CAPTURE-3C- | CAPTURE- | K562 FB-dCas9/BirA clone6 | GSM2635075 | This study |
| seq_K562_sgHS3- | 3C-seq | | | |
| combined | | | | |
| CAPTURE-3C- | CAPTURE- | K562 FB-dCas9/BirA clone6 | GSM2635076 | This study |
| seq_K562_sgHS4-rep1 | 3C-seq | | | |
| CAPTURE-3C- | CAPTURE- | K562 FB-dCas9/BirA clone6 | GSM2635077 | This study |
| seq_K562_sgHS4-rep2 | 3C-seq | | | |
| CAPTURE-3C- | CAPTURE- | K562 FB-dCas9/BirA clone6 | GSM2635078 | This study |
| seq_K562_sgHS4- | 3C-seq | | | |
| combined | | | | |
| CAPTURE-3C- | CAPTURE- | K562 FB-dCas9/BirA clone6 | GSM2635083 | This study |
| seq_K562_sgHBB-rep1 | 3C-seq | | | |
| CAPTURE-3C- | CAPTURE- | K562 FB-dCas9/BirA clone6 | GSM2635084 | This study |
| seq_K562_sgHBB-rep2 | 3C-seq | | | |
| CAPTURE-3C- | CAPTURE- | K562 FB-dCas9/BirA clone6 | GSM2635085 | This study |
| seq_K562_sgHBB-rep3 | 3C-seq | | | |
| CAPTURE-3C- | CAPTURE- | K562 FB-dCas9/BirA clone6 | GSM2635086 | This study |
| seq_K562_sgHBB- | 3C-seq | | | |
| combined | | | | |
| CAPTURE-3C- | CAPTURE- | K562 FB-dCas9/BirA clone6 | GSM2635087 | This study |
| seq_K562_sgHBG-rep1 | 3C-seq | | | |
| CAPTURE-3C- | CAPTURE- | K562 FB-dCas9/BirA clone6 | GSM2635088 | This study |
| seq_K562_sgHBG-rep2 | 3C-seq | | | |
| CAPTURE-3C- | CAPTURE- | K562 FB-dCas9/BirA clone6 | GSM2635089 | This study |
| seq_K562_sgHBG-rep3 | 3C-seq | | | |
| CAPTURE-3C- | CAPTURE- | K562 FB-dCas9/BirA clone6 | GSM2635090 | This study |
| seq_K562_sgHBG- | 3C-seq | | | |
| combined | | | | |
| CAPTURE-3C- | CAPTURE- | K562 FB-dCas9/BirA clone6 | GSM2635091 | This study |
| seq_K562_sgHBD-1kb- | 3C-seq | | | |
| rep1 | | | | |
| CAPTURE-3C- | CAPTURE- | K562 FB-dCas9/BirA clone6 | GSM2635092 | This study |
| seq_K562_sgHBD-1kb- | 3C-seq | | | |
| rep2 | | | | |
| CAPTURE-3C- | CAPTURE- | K562 FB-dCas9/BirA clone6 | GSM2635093 | This study |
| seq_K562_sgHBD-1kb- | 3C-seq | | | |
| rep3 | | | | |
| CAPTURE-3C- | CAPTURE- | K562 FB-dCas9/BirA clone6 | GSM2635094 | This study |
| seq_K562_sgHBD-1kb- | 3C-seq | | | |
| combined | | | | |
| CAPTURE-3C- | CAPTURE- | K562 FB-dCas9/BirA clone6 | GSM2635095 | This study |
| seq_K562_sgHBD- | 3C-seq | | | |
| 1.5kb-rep1 | | | | |
| CAPTURE-3C- | CAPTURE- | K562 FB-dCas9/BirA clone6 | GSM2635096 | This study |
| seq_K562_sgHBD- | 3C-seq | | | |
| 1.5kb-rep2 | | | | |
| CAPTURE-3C- | CAPTURE- | K562 FB-dCas9/BirA clone6 | GSM2635097 | This study |
| seq_K562_sgHBD- | 3C-seq | | | |
| 1.5kb-combined | | | | |
| CAPTURE-3C- | CAPTURE- | K562 FB-dCas9/BirA clone6 | GSM2635098 | This study |
| seq_K562_sgHBD-2kb- | 3C-seq | | | |
| rep1 | | | | |
| CAPTURE-3C- | CAPTURE- | K562 FB-dCas9/BirA clone6 | GSM2635099 | This study |
| seq_K562_sgHBD-2kb- | 3C-seq | | | |
| rep2 | | | | |
| CAPTURE-3C- | CAPTURE- | K562 FB-dCas9/BirA clone6 | GSM2635100 | This study |
| seq_K562_gHBD-2kb- | 3C-seq | | | |
| combined | | | | |
| CAPTURE-3C- | CAPTURE- | K562 FB-dCas9/BirA clone6 | GSM2635101 | This study |
| seq_K562_sg3′HS1- | 3C-seq | | | |
| rep1 | | | | |
| CAPTURE-3C- | CAPTURE- | K562 FB-dCas9/BirA clone6 | GSM2635102 | This study |
| seq_K562_sg3′HS1- | 3C-seq | | | |
| rep2 | | | | |
| CAPTURE-3C- | CAPTURE- | K562 FB-dCas9/BirA clone6 | GSM2635103 | This study |
| seq_K562_sg3′HS1- | 3C-seq | | | |
| rep3 | | | | |
| CAPTURE-3C- | CAPTURE- | K562 FB-dCas9/BirA clone6 | GSM2635104 | This study |
| seq_K562_sg3′HS1- | 3C-seq | | | |
| combined | | | | |
| CAPTURE-3C- | CAPTURE- | K562 FB-dCas9/BirA clone6 | GSM2635105 | This study |
| seq_K562_sgGal4_no_capture_control | 3C-seq | | | |
| CAPTURE-3C- | CAPTURE- | K562 FB-dCas9/BirA clone6 | GSM2635106 | This study |
| seq_K562_gDNA_control | 3C-seq | | | |
| CAPTURE-3C- | CAPTURE- | K562 FB-dCas9/BirA clone6 | GSM2635107 | This study |
| seq_K562_sgHS2-sg3- | 3C-seq | | | |
| rep1 | | | | |
| CAPTURE-3C- | CAPTURE- | K562 FB-dCas9/BirA clone6 | GSM2635108 | This study |
| seq_K562_sgHS2-sg3- | 3C-seq | | | |
| rep2 | | | | |
| CAPTURE-3C- | CAPTURE- | K562 FB-dCas9/BirA clone6 | GSM2635109 | This study |
| seq_K562_sgHS2-sg3- | 3C-seq | | | |
| rep3 | | | | |
| CAPTURE-3C- | CAPTURE- | K562 FB-dCas9/BirA clone6 | GSM2635110 | This study |
| seq_K562_sgHS2-sg3- | 3C-seq | | | |
| combined | | | | |
| CAPTURE-3C- | CAPTURE- | K562 FB-dCas9/BirA clone6 | GSM2635111 | This study |
| seq_K562_sgHS2-sg4- | 3C-seq | | | |
| rep1 | | | | |
| CAPTURE-3C- | CAPTURE- | K562 FB-dCas9/BirA clone6 | GSM2635112 | This study |
| seq_K562_sgHS2-sg4- | 3C-seq | | | |
| rep2 | | | | |
| CAPTURE-3C- | CAPTURE- | K562 FB-dCas9/BirA clone6 | GSM2635113 | This study |
| seq_K562_sgHS2-sg4- | 3C-seq | | | |
| rep3 | | | | |
| CAPTURE-3C- | CAPTURE- | K562 FB-dCas9/BirA clone6 | GSM2635114 | This study |
| seq_K562_sgHS2-sg4- | 3C-seq | | | |
| combined | | | | |
| CAPTURE-3C- | CAPTURE- | K562 FB-dCas9/BirA clone6 | GSM2635115 | This study |
| seq_K562_sgHS2-sg5- | 3C-seq | | | |
| rep1 | | | | |
| CAPTURE-3C- | CAPTURE- | K562 FB-dCas9/BirA clone6 | GSM2635116 | This study |
| seq_K562_sgHS2-sg5- | 3C-seq | | | |
| rep2 | | | | |
| CAPTURE-3C- | CAPTURE- | K562 FB-dCas9/BirA clone6 | GSM2635117 | This study |
| seq_K562_sgHS2-sg5- | 3C-seq | | | |
| rep3 | | | | |
| CAPTURE-3C- | CAPTURE- | K562 FB-dCas9/BirA clone6 | GSM2635118 | This study |
| seq_K562_sgHS2-sg5- | 3C-seq | | | |
| combined | | | | |
| CAPTURE-3C- | CAPTURE- | K562 FB-dCas9/BirA clone6 | GSM2635119 | This study |
| seq_K562_sgHS3-sg2- | 3C-seq | | | |
| rep1 | | | | |
| CAPTURE-3C- | CAPTURE- | K562 FB-dCas9/BirA clone6 | GSM2635120 | This study |
| seq_K562_sgHS3-sg2- | 3C-seq | | | |
| rep2 | | | | |
| CAPTURE-3C- | CAPTURE- | K562 FB-dCas9/BirA clone6 | GSM2635121 | This study |
| seq_K562_sgHS3-sg2- | 3C-seq | | | |
| rep3 | | | | |
| CAPTURE-3C- | CAPTURE- | K562 FB-dCas9/BirA clone6 | GSM2635122 | This study |
| seq_K562_sgHS3-sg2- | 3C-seq | | | |
| combined | | | | |
| CAPTURE-3C- | CAPTURE- | K562 FB-dCas9/BirA clone6 | GSM2635123 | This study |
| seq_K562_sgHS3-sg3- | 3C-seq | | | |
| rep1 | | | | |
| CAPTURE-3C- | CAPTURE- | K562 FB-dCas9/BirA clone6 | GSM2635124 | This study |
| seq_K562_sgHS3-sg3- | 3C-seq | | | |
| rep2 | | | | |
| CAPTURE-3C- | CAPTURE- | K562 FB-dCas9/BirA clone6 | GSM2635125 | This study |
| seq_K562_sgHS3-sg3- | 3C-seq | | | |
| combined | | | | |
| CAPTURE-3C- | CAPTURE- | K562 FB-dCas9/BirA clone6 | GSM2635126 | This study |
| seq_K562_sgHS3-sg4- | 3C-seq | | | |
| rep1 | | | | |
| CAPTURE-3C- | CAPTURE- | K562 FB-dCas9/BirA clone6 | GSM2635127 | This study |
| seq_K562_sgHS3-sg4- | 3C-seq | | | |
| rep2 | | | | |
| CAPTURE-3C- | CAPTURE- | K562 FB-dCas9/BirA clone6 | GSM2635128 | This study |
| seq_K562_sgHS3-sg4- | 3C-seq | | | |
| rep3 | | | | |
| CAPTURE-3C- | CAPTURE- | K562 FB-dCas9/BirA clone6 | GSM2635129 | This study |
| seq_K562_sgHS3-sg4- | 3C-seq | | | |
| combined | | | | |
| CAPTURE-3C- | CAPTURE- | K562 FB-dCas9/BirA clone6 | GSM2635130 | This study |
| seq_K562_HBD- | 3C-seq | | | |
| 1k_Del_sgHS3-rep1 | | | | |
| CAPTURE-3C- | CAPTURE- | K562 FB-dCas9/BirA clone6 | GSM2635131 | This study |
| seq_K562_HBD- | 3C-seq | | | |
| 1k_Del_sgHS3-rep2 | | | | |
| CAPTURE-3C- | CAPTURE- | K562 FB-dCas9/BirA clone6 | GSM2635132 | This study |
| seq_K562_HBD- | 3C-seq | | | |
| 1k_Del_sgHS3- | | | | |
| combined | | | | |
| CAPTURE-3C- | CAPTURE- | K562 FB-dCas9/BirA clone6 | GSM2635133 | This study |
| seq_K562_HBD- | 3C-seq | | | |
| 1k_Del_sg3-HS1-rep1 | | | | |
| CAPTURE-3C- | CAPTURE- | K562 FB-dCas9/BirA clone6 | GSM2635134 | This study |
| seq_K562_HBD- | 3C-seq | | | |
| 1k_Del_sg3-HS1-rep2 | | | | |
| CAPTURE-3C- | CAPTURE- | K562 FB-dCas9/BirA clone6 | GSM2635135 | This study |
| seq_K562_HBD- | 3C-seq | | | |
| 1k_Del_sg3-HS1- | | | | |
| combined | | | | |
| CAPTURE-3C- | CAPTURE- | KH2 FB-dCas9-IRES-BirA | GSM2635136 | This study |
| seq_ESC-KH2_sgEsrrb- | 3C-seq | clone5 | | |
| SE | | | | |
| CAPTURE-3C- | CAPTURE- | KH2 FB-dCas9-IRES-BirA | GSM2635137 | This study |
| seq_ESC-KH2_sgUtf-1- | 3C-seq | clone5 | | |
| SE | | | | |
| CAPTURE-3C- | CAPTURE- | KH2 FB-dCas9-IRES-BirA | GSM2635138 | This study |
| seq_ESC-KH2_sgOct4- | 3C-seq | clone5 | | |
| SE | | | | |
| CAPTURE-3C- | CAPTURE- | KH2 FB-dCas9-IRES-BirA | GSM2635139 | This study |
| seq_ESC-KH2_sgSox2- | 3C-seq | clone5 | | |
| SE | | | | |
| CAPTURE-3C-seq_EB- | CAPTURE- | KH2 FB-dCas9-IRES-BirA | GSM2635140 | This study |
| KH2_sgEsrrb-SE | 3C-seq | clone5 | | |
| CAPTURE-3C-seq_EB- | CAPTURE- | KH2 FB-dCas9-IRES-BirA | GSM2635141 | This study |
| KH2_sgUtf-1-SE | 3C-seq | clone5 | | |
| CAPTURE-3C-seq_EB- | CAPTURE- | KH2 FB-dCas9-IRES-BirA | GSM2635142 | This study |
| KH2_sgOct4-SE | 3C-seq | clone5 | | |
| CAPTURE-3C-seq_EB- | CAPTURE- | KH2 FB-dCas9-IRES-BirA | GSM2635143 | This study |
| KH2_sgSox2-SE | 3C-seq | clone5 | | |
| RNA-seq_K562_FB- | RNA-seq | K562 FB-dCas9/BirA clone6 | GSM2635259 | This study |
| dCas9-BirA_sgGal4- | | | | |
| rep1 | | | | |
| RNA-seq_K562_FB- | RNA-seq | K562 FB-dCas9/BirA Clone6 | GSM2635260 | This study |
| dCas9-BirA_sgGal4- | | | | |
| rep2 | | | | |
| RNA-seq_K562_FB- | RNA-seq | K562 FB-dCas9/BirA clone6 | GSM2635261 | This study |
| dCas9-BirA_sgGal4- | | | | |
| rep3 | | | | |
| RNA-seq_K562_FB- | RNA-seq | K562 FB-dCas9/BirA clone6 | GSM2635262 | This study |
| dCas9-BirA_sgHBG- | | | | |
| rep1 | | | | |
| RNA-seq_K562_FB- | RNA-seq | K562 FB-dCas9/BirA clone6 | GSM2635263 | This study |
| dCas9-BirA_sgHBG- | | | | |
| rep2 | | | | |
| RNA-seq_K562_FB- | RNA-seq | K562 FB-dCas9/BirA clone6 | GSM2635264 | This study |
| dCas9-BirA_sgHBG- | | | | |
| rep3 | | | | |
| RNA-seq_K562_FB- | RNA-seq | K562 FB-dCas9/BirA clone6 | GSM2635265 | This study |
| dCas9-BirA_sgHS2- | | | | |
| rep1 | | | | |
| RNA-seq_K562_FB- | RNA-seq | K562 FB-dCas9/BirA clone6 | GSM2635266 | This study |
| dCas9-BirA_sgHS2- | | | | |
| rep2 | | | | |
| RNA-seq_K562_FB- | RNA-seq | K562 FB-dCas9/BirA clone6 | GSM2635267 | This study |
| dCas9-BirA_sgHS2- | | | | |
| rep3 | | | | |
| RNA-seq_K562_FB- | RNA-seq | K562 FB-dCas9/BirA clone6 | GSM2635268 | This study |
| dCas9-BirA_sgHS1-5- | | | | |
| rep1 | | | | |
| RNA-seq_K562_FB- | RNA-seq | K562 FB-dCas9/BirA clone6 | GSM2635269 | This study |
| dCas9-BirA_sgHS1-5- | | | | |
| rep2 | | | | |
| RNA-seq_K562_FB- | RNA-seq | K562 FB-dCas9/BirA clone6 | GSM2635270 | This study |
| dCas9-BirA_sgHS1-5- | | | | |
| rep3 | | | | |
| RNA-seq_K562_WT- | RNA-seq | K562 | GSM2635271 | This study |
| rep1 | | | | |
| RNA-seq_K562_WT- | RNA-seq | K562 | GSM2635272 | This study |
| rep2 | | | | |
| ATAC- | ATAC-seq | K562 | GSM2695560 | This study |
| seq_K562_Control-rep1 | | | | |
| ATAC- | ATAC-seq | K562 | GSM2695561 | This study |
| seq_K562_Control-rep2 | | | | |
| ATAC- | ATAC-seq | K562 | GSM2695562 | This study |
| seq_K562_Control-rep3 | | | | |
| ATAC- | ATAC-seq | K562-HBD-1K_Del_Clone12 | GSM2695563 | This study |
| seq_K562_HBD- | | | | |
| 1kb_KO-rep1 | | | | |
| ATAC- | ATAC-seq | K562-HBD-1K_Del_Clone14 | GSM2695564 | This study |
| seq_K562_HBD- | | | | |
| 1kb_KO-rep2 | | | | |
| ATAC- | ATAC-seq | K562-HBD-1K_Del_Clone48 | GSM2695565 | This study |
| seq_K562_HBD- | | | | |
| 1kb_KO-rep3 | | | | |
| ATAC-seq_ESC- | ATAC-seq | KH2 FB-dCas9-IRES-BirA | GSM2695566 | This study |
| KH2_rep1 | | clone5 | | |
| ATAC-seq_ESC- | ATAC-seq | KH2 FB-dCas9-IRES-BirA | GSM2695567 | This study |
| KH2_rep2 | | Clone5 | | |
| ATAC-seq_ESC- | ATAC-seq | KH2 FB-dCas9-IRES-BirA | GSM2695568 | This study |
| KH2_rep3 | | clone5 | | |
| ATAC-seq_ESC- | ATAC-seq | KH2 FB-dCas9-IRES-BirA | GSM2695569 | This study |
| KH2_rep4 | | clone5 | | |
| ATAC-seq_EB- | ATAC-seq | KH2 FB-dCas9-IRES-BirA | GSM2695570 | This study |
| KH2_rep1 | | clone5 | | |
| ATAC-seq_EB- | ATAC-seq | KH2 FB-dCas9-IRES-BirA | GSM2695571 | This study |
| KH2_rep2 | | clone5 | | |
| ATAC-seq_EB- | ATAC-seq | KH2 FB-dCas9-IRES-BirA | GSM2695572 | This study |
| KH2_rep3 | | clone5 | | |
| ATAC-seq_EB- | ATAC-seq | KH2 FB-dCas9-IRES-BirA | GSM2695573 | This study |
| KH2_rep4 | | clone5 | | |
| ChIP- | ChIP-seq | K562 | GSM2635249 | This study |
| seq_K562_DMSO_BRD4 | | | | |
| ChIP- | ChIP-seq | K562 | GSM2635250 | This study |
| seq_K562_DMSO_RNAPII | | | | |
| ChIP-seq_K562_JQ1- | ChIP-seq | K562 | GSM2635251 | This study |
| 2h_BRD4 | | | | |
| ChIP-seq_K562_JQ1- | ChIP-seq | K562 | GSM2635252 | This study |
| 2h_RNAPII | | | | |
| ChIP-seq_K562_JQ1- | ChIP-seq | K562 | GSM2635253 | This study |
| 6h_BRD4 | | | | |
| ChIP-seq_K562_JQ1- | ChIP-seq | K562 | GSM2635254 | This study |
| 6h_RNAPII | | | | |
| ChIP- | ChIP-seq | K562 | GSM2635255 | This study |
| seq_K562_NUP98 | | | | |
| ChIP- | ChIP-seq | K562 | GSM2635256 | This study |
| seq_K562_NUP153 | | | | |
| ChIP-seq_ESC- | ChIP-seq | KH2 FB-dCas9-IRES-BirA | GSM2695589 | This study |
| KH2_H3K27ac-rep1 | | clone5 | | |
| ChIP-seq_ESC- | ChIP-seq | KH2 FB-dCas9-IRES-BirA | GSM2695590 | This study |
| KH2_H3K27ac-rep2 | | clone5 | | |
| ChIP-seq_EB- | ChIP-seq | KH2 FB-dCas9-IRES-BirA | GSM2695591 | This study |
| KH2_H3K27ac | | clone5 | | |
| ChIP- | ChIP-seq | K562 | GSM2309710 | Liu X, et al. |
| seq_K562_H3K27ac | | | | Nature Cell |
| | | | | Biology |
| | | | | 2017 |
| ChIP- | ChIP-seq | K562 | GSM1003608 | Pope BD, et |
| seq_K562_GATA1 | | | | al. Nature |
| | | | | 2014 |
| ChIP- | ChIP-seq | K562 | GSM1003625 | Pope BD, et |
| seq_K562_HCFC1 | | | | al. Nature |
| | | | | 2014 |
| ChIP- | ChIP-seq | K562 | GSM1003448 | ENCODE |
| seq_K562_HDAC1 | | | | Project |
| | | | | Consortium, |
| | | | | et al. Nature |
| | | | | 2012 |
| ChIP- | ChIP-seq | K562 | GSM803471 | Gertz J, et |
| seq_K562_HDAC2 | | | | al. Mol. Cell |
| | | | | 2013 |
| ChIP- | ChIP-seq | K562 | GSM822275 | Pope BD, et |
| seq_K562_POLR2A | | | | al. Nature |
| | | | | 2014 |
| ChIP- | ChIP-seq | K562 | GSM935633 | Pope BD, et |
| seq_K562_SMARCA4 | | | | al. Nature |
| | | | | 2014 |
| ChIP- | ChIP-seq | K562 | GSM935487 | ENCODE |
| seq_K562_STAT1 | | | | Project |
| | | | | Consortium, |
| | | | | et al. Nature |
| | | | | 2012 |
| ChIP- | ChIP-seq | K562 | GSM1010877 | Gertz J, et |
| seq_K562_STAT5A | | | | al. Mol. Cell |
| | | | | 2013 |
| ChIP- | ChIP-seq | K562 | GSM935574 | Pope BD, et |
| seq_K562_TBL1XR1 | | | | al. Nature |
| | | | | 2014 |
| ChIP- | ChIP-seq | K562 | GSM1010849 | Gertz J, et |
| seq_K562_TRIM28 | | | | al. Mol. Cell |
| | | | | 2013 |
| ChIP- | ChIP-seq | K562 | GSM1003492 | ENCODE |
| seq_K562_WHSC1 | | | | Project |
| | | | | Consortium, |
| | | | | et al. Nature |
| | | | | 2012 |
| ChIP- | ChIP-seq | K562 | GSM803504 | Gertz J, et |
| seq_K562_ZBTB33 | | | | al. Mol. Cell |
| | | | | 2013 |
| ChIP-seq_K562_TAL1 | ChIP-seq | K562 | GSM935496 | Pope BD, et |
| | | | | al. Nature |
| | | | | 2014 |
| ChIA- | ChIA-PET | K562 | GSM970213 | ENCODE |
| PET_K562_RNAPII | | | | Project |
| | | | | Consortium, |
| | | | | et al. Nature |
| | | | | 2012 |
| ChIA- | ChIA-PET | K562 | GSM970216 | ENCODE |
| PET_K562_CTCF | | | | Project |
| | | | | Consortium, |
| | | | | et al. Nature |
| | | | | 2012 |
| UMI-4C_K562 | UMI-4C | K562 | GSM2037371 | Schwartzman O, |
| | | | | et al. |
| | | | | Nature |
| | | | | Methods |
| | | | | 2016 |
| 5C_K562 | 5C | K562 | GSM970500 | Naumova N, |
| | | | | et al. |
| | | | | Science |
| | | | | 2013 |
| In Situ Hi-C_K562 | In Situ Hi-C | K562 | GSM1551618 | Rao S, et al. |
| | | | | Cell 2014 |
| Genome- | DNase Hi-C | K562 | GSM1370434 | Ma W, et al. |
| Wide_DNase_Hi- | | | | Nature |
| C_K562 | | | | Methods |
| | | | | 2015 |
| LCR- | DNase Hi-C | K562 | GSM1370436 | Ma W, et al. |
| Targeted_DNase_Hi- | | | | Nature |
| C_K562 | | | | Methods |
| | | | | 2015 |

The peripheral NUPs including NUP98, NUP153 and NUP214 extend from the membrane-embedded NPC scaffold to regulate nuclear trafficking. While a few NUPs were found to be associated with transcriptionally active genes or regulatory elements (Capelson et al., 2010; Ibarra et al., 2016; Kalverda et al., 2010), their roles in erythroid enhancers remained unknown. Hence, the inventors performed NUP98 and NUP153 ChIP-seq in K562 cells, and identified 5,283 and 4,996 binding sites in gene-proximal promoters and distal elements (FIG. 4C). Notably, NUP98 and NUP153 binding sites are highly enriched at erythroid SEs (FIGS. 4D,4E), associated with gene activation (FIG. 4F), nucleosome organization and DNA packaging (FIG. 4G), highlighting their potential roles in regulating chromatin organization and/or enhancer activities. Moreover, NUP98/NUP153 binding sites are enriched for motifs associated with hematopoietic TFs, chromatin factors and homeobox proteins (FIG. 4H), suggesting that NUPs may cooperate with lineage TFs and chromatin regulators in gene transcription. Another identified protein BRD4 binds acetylated histones and plays a critical role in chromatin regulation. Inhibition of BRD4 by a small molecule JQ1 abrogates its function (Filippakopoulos et al., 2010). BRD4 and related BET proteins (BRD2 and BRD3) are required for globin gene transcription in mouse erythroid cells (Stonestrom et al., 2015). Consistently, inhibition of BET proteins by JQ1 in human erythroid cells significantly decreased β-globin mRNAs and BRD4 occupancy without apparent effects on erythroid differentiation (FIGS. 10C-10F). Together, these results not only establish new regulators of β-globin enhancers, but demonstrate the potential of the CAPTURE approach for unambiguous identification of protein complexes specifically associated with a single genomic locus, such as an enhancer, in situ.

TABLE 2
Sequences of sgRNAs, shRNAs
and Primers, Related STAR Methods.

| | | | SEQ | |
|---|---|---|---|---|
| Name | Forward | Reverse | ID NO | Application |
| sgHBG_sg1 | ggagaaCCACCTTGTTGGCT | ctagtaCTCGAGAAAAAAAGC | 1, 2 | Primers used |
| | AAACTCCACCCATGGGTG | ACCGACTCGGTGCCAC | | to clone |
| | TTTAAGAGCTATGCTGGA | | | sgRNA into |
| | AACAGCA | | | pSLQ1681 |
| sgHBG_sg2 | ggagaaCCACCTTGTTGGCT | ctagtaCTCGAGAAAAAAAGC | 3, 4 | for |
| | CCTAGTCCAGACGCCATG | ACCGACTCGGTGCCAC | | CAPTURE |
| | TTTAAGAGCTATGCTGGA | | | targeting |
| | AACAGCA | | | |
| sgHBB_sg1 | ggagaaCCACCTTGTTGGCC | ctagtaCTCGAGAAAAAAAGC | 5, 6 | |
| | CTGTGGAGCCACACCCTA | ACCGACTCGGTGCCAC | | |
| | GTTTAAGAGCTATGCTGG | | | |
| | AAACAGCA | | | |
| sgHBB_sg2 | ggagaaCCACCTTGTTGGTC | ctagtaCTCGAGAAAAAAAGC | 7, 8 | |
| | TGCCGTTACTGCCCTGTG | ACCGACTCGGTGCCAC | | |
| | TTTAAGAGCTATGCTGGA | | | |
| | AACAGCA | | | |
| sgHS1_sg1 | ggagaaCCACCTTGTTGGCA | ctagtaCTCGAGAAAAAAAGC | 9, 10 | |
| | ATAGGTATATGAGGAGA | ACCGACTCGGTGCCAC | | |
| | CGTTTAAGAGCTATGCTG | | | |
| | GAAACAGCA | | | |
| sgHS1_sg2 | ggagaaCCACCTTGTTGGTT | ctagtaCTCGAGAAAAAAAGC | 11, 12 | |
| | GTGTAGAAACCAAGCGT | ACCGACTCGGTGCCAC | | |
| | GGTTTAAGAGCTATGCTG | | | |
| | GAAACAGCA | | | |
| sgHS2_sg1 | ggagaaCCACCTTGTTGGAG | ctagtaCTCGAGAAAAAAAGC | 13, 14 | |
| | TCCAAGCATGAGCAGTTC | ACCGACTCGGTGCCAC | | |
| | GTTTAAGAGCTATGCTGG | | | |
| | AAACAGCA | | | |
| sgHS2_sg2 | ggagaaCCACCTTGTTGGTG | ctagtaCTCGAGAAAAAAAGC | 15, 16 | |
| | GCCTCTATACCTAGAAGG | ACCGACTCGGTGCCAC | | |
| | TTTAAGAGCTATGCTGGA | | | |
| | AACAGCA | | | |
| sgHS2_sg3 | ggagaaCCACCTTGTTGGTA | ctagtaCTCGAGAAAAAAAGC | 17, 18 | |
| | TAATGTGCTCTGTCCCCC | ACCGACTCGGTGCCAC | | |
| | GTTTAAGAGCTATGCTGG | | | |
| | AAACAGCA | | | |
| sgHS2_sg4 | ggagaaCCACCTTGTTGGAA | ctagtaCTCGAGAAAAAAAGC | 19, 20 | |
| | TAGTGTTTAGCATCCAGC | ACCGACTCGGTGCCAC | | |
| | GTTTAAGAGCTATGCTGG | | | |
| | AAACAGCA | | | |
| sgHS2_sg5 | ggagaaCCACCTTGTTGCTT | ctagtaCTCGAGAAAAAAAGC | 21, 22 | |
| | TATGATGCCGTTTGAGGG | ACCGACTCGGTGCCAC | | |
| | TTTAAGAGCTATGCTGGA | | | |
| | AACAGCA | | | |
| sgHS3_sg1 | ggagaaCCACCTTGTTGGAG | ctagtaCTCGAGAAAAAAAGC | 23, 24 | |
| | AGATAGACCATGAGTAG | ACCGACTCGGTGCCAC | | |
| | GTTTAAGAGCTATGCTGG | | | |
| | AAACAGCA | | | |
| sgHS3_sg2 | ggagaaCCACCTTGTTGGAG | ctagtaCTCGAGAAAAAAAGC | 25, 26 | |
| | GAATCATTCTGTGGATAA | ACCGACTCGGTGCCAC | | |
| | GTTTAAGAGCTATGCTGG | | | |
| | AAACAGCA | | | |
| sgHS3_sg3 | ggagaaCCACCTTGTTGGAA | ctagtaCTCGAGAAAAAAAGC | 27, 28 | |
| | GTCTATGACTGTAAATTG | ACCGACTCGGTGCCAC | | |
| | TTTAAGAGCTATGCTGGA | | | |
| | AACAGCA | | | |
| sgHS3_sg4 | ggagaaCCACCTTGTTGGCC | ctagtaCTCGAGAAAAAAAGC | 29, 30 | |
| | CCTAGCTGGGGGTATAGG | ACCGACTCGGTGCCAC | | |
| | TTTAAGAGCTATGCTGGA | | | |
| | AACAGCA | | | |
| sgHS4_sg1 | ggagaaCCACCTTGTTGGCC | ctagtaCTCGAGAAAAAAAGC | 31, 32 | |
| | CACTCAGCAGCTATGAGA | ACCGACTCGGTGCCAC | | |
| | GTTTAAGAGCTATGCTGG | | | |
| | AAACAGCA | | | |
| sgHS4_sg2 | ggagaaCCACCTTGTTGGTC | ctagtaCTCGAGAAAAAAAGC | 33, 34 | |
| | TCCCTCCCATTCCCGAGC | ACCGACTCGGTGCCAC | | |
| | GTTTAAGAGCTATGCTGG | | | |
| | AAACAGCA | | | |
| sgHS5_sg1 | ggagaaCCACCTTGTTGGTG | ctagtaCTCGAGAAAAAAAGC | 35, 36 | |
| | CCCCCACCTTACAGGGAC | ACCGACTCGGTGCCAC | | |
| | GTTTAAGAGCTATGCTGG | | | |
| | AAACAGCA | | | |
| sgHS5_sg2 | ggagaaCCACCTTGTTGGGA | ctagtaCTCGAGAAAAAAAGC | 37, 38 | |
| | GCCCTTTTGATTGAAGGG | ACCGACTCGGTGCCAC | | |
| | TTTAAGAGCTATGCTGGA | | | |
| | AACAGCA | | | |
| 3HS1_sgRNA | ggagaaCCACCTTGTTGGCT | ctagtaCTCGAGAAAAAAAGC | 39, 40 | |
| | TTAGTGTAAGCGAGGTCG | ACCGACTCGGTGCCAC | | |
| | TTTAAGAGCTATGCTGGA | | | |
| | AACAGCA | | | |
| HBD-1kb_sgRNA1 | ggagaaCCACCTTGTTGGTA | ctagtaCTCGAGAAAAAAAGC | 41, 42 | |
| | CAATAGTATAACCCCTTG | ACCGACTCGGTGCCAC | | |
| | TTTAAGAGCTATGCTGGA | | | |
| | AACAGCA | | | |
| HBD-1.5kb_sgRNA | ggagaaCCACCTTGTTGGCT | ctagtaCTCGAGAAAAAAAGC | 43, 44 | |
| | GGGCTTCTGTTGCAGTAG | ACCGACTCGGTGCCAC | | |
| | TTTAAGAGCTATGCTGGA | | | |
| | AACAGCA | | | |
| HBD-2kb_sgRNA | ggagaaCCACCTTGTTGGAG | ctagtaCTCGAGAAAAAAAGC | 45, 46 | |
| | ATCAAATAACAGTCCTCA | ACCGACTCGGTGCCAC | | |
| | GTTTAAGAGCTATGCTGG | | | |
| | AAACAGCA | | | |
| Telomere-sgRNA | ggagaaCCACCTTGTTGGTT | ctagtaCTCGAGAAAAAAAGC | 47, 48 | |
| | AGGGTTAGGGTTAGGGTT | ACCGACTCGGTGCCAC | | |
| | AGTTTAAGAGCTATGCTG | | | |
| | GAAACAGCA | | | |
| GAL4_sgRNA | ggagaaCCACCTTGTTGGAA | ctagtaCTCGAGAAAAAAAGC | 49, 50 | |
| | CGACTAGTTAGGCGTGTA | ACCGACTCGGTGCCAC | | |
| | GTTTAAGAGCTATGCTGG | | | |
| | AAACAGCA | | | |
| Esrrb_SE1_sgRNA1.fwd | ggagaaCCACCTTGTTGGTT | ctagtaCTCGAGAAAAAAAGC | 51, 52 | |
| | TCTCTATGAAGTGAAGCG | ACCGACTCGGTGCCAC | | |
| | TTTAAGAGCTATGCTGGA | | | |
| | AACAGCA | | | |
| Esrrb_SE1_sgRNA2.fwd | ggagaaCCACCTTGTTGgCT | ctagtaCTCGAGAAAAAAAGC | 53, 54 | |
| | CTCTACCCTCGGGGCGAT | ACCGACTCGGTGCCAC | | |
| | GTTTAAGAGCTATGCTGG | | | |
| | AAACAGCA | | | |
| Esrrb_SE1_sgRNA3.fwd | ggagaaCCACCTTGTTGgCT | ctagtaCTCGAGAAAAAAAGC | 55, 56 | |
| | CAAACTATGCCCACCTGC | ACCGACTCGGTGCCAC | | |
| | GTTTAAGAGCTATGCTGG | | | |
| | AAACAGCA | | | |
| Esrrb_SE1_sgRNA4.fwd | ggagaaCCACCTTGTTGGGA | ctagtaCTCGAGAAAAAAAGC | 57, 58 | |
| | CTTGAAAGATGCAGGGG | ACCGACTCGGTGCCAC | | |
| | GTTTAAGAGCTATGCTGG | | | |
| | AAACAGCA | | | |
| Esrrb_SE2_sgRNA1.fwd | ggagaaCCACCTTGTTGgTA | ctagtaCTCGAGAAAAAAAGC | 59, 60 | |
| | CTAATTAACTTATAGTTG | ACCGACTCGGTGCCAC | | |
| | GTTTAAGAGCTATGCTGG | | | |
| | AAACAGCA | | | |
| Esrrb_SE2_sgRNA2.fwd | ggagaaCCACCTTGTTGgCA | ctagtaCTCGAGAAAAAAAGC | 61, 62 | |
| | AAGGATGAATGTGTCGAC | ACCGACTCGGTGCCAC | | |
| | GTTTAAGAGCTATGCTGG | | | |
| | AAACAGCA | | | |
| Esrrb_SE3_sgRNA1.fwd | ggagaaCCACCTTGTTGgCA | ctagtaCTCGAGAAAAAAAGC | 63, 64 | |
| | CAAGGCTATAATGAACGC | ACCGACTCGGTGCCAC | | |
| | GTTTAAGAGCTATGCTGG | | | |
| | AAACAGCA | | | |
| Esrrb_SE3_sgRNA2.fwd | ggagaaCCACCTTGTTGGAG | ctagtaCTCGAGAAAAAAAGC | 65, 66 | |
| | AGTTTTCCTAGCGCAGAG | ACCGACTCGGTGCCAC | | |
| | TTTAAGAGCTATGCTGGA | | | |
| | AACAGCA | | | |
| Esrrb_SE3_sgRNA3.fwd | ggagaaCCACCTTGTTGgTA | ctagtaCTCGAGAAAAAAAGC | 67, 68 | |
| | AGAGTCGAGTATTGGCGA | ACCGACTCGGTGCCAC | | |
| | GTTTAAGAGCTATGCTGG | | | |
| | AAACAGCA | | | |
| Utf1_SE1_sgRNA1.fwd | ggagaaCCACCTTGTTGGAG | ctagtaCTCGAGAAAAAAAGC | 69, 70 | |
| | CGGCGGCGAACCCTCGG | ACCGACTCGGTGCCAC | | |
| | GTTTAAGAGCTATGCTGG | | | |
| | AAACAGCA | | | |
| Utf1_SE1_sgRNA2.fwd | ggagaaCCACCTTGTTGgCT | ctagtaCTCGAGAAAAAAAGC | 71, 72 | |
| | GGGCTTTGCTAAGTCCGT | ACCGACTCGGTGCCAC | | |
| | GTTTAAGAGCTATGCTGG | | | |
| | AAACAGCA | | | |
| Utf1_SE1_sgRNA3.fwd | ggagaaCCACCTTGTTGgTC | ctagtaCTCGAGAAAAAAAGC | 73, 74 | |
| | TCTCACAGAAGGGATCGC | ACCGACTCGGTGCCAC | | |
| | GTTTAAGAGCTATGCTGG | | | |
| | AAACAGCA | | | |
| Utf1_SE1_sgRNA4.fwd | ggagaaCCACCTTGTTGgTTT | ctagtaCTCGAGAAAAAAAGC | 75, 76 | |
| | CCCCTAGACAATGACGGG | ACCGACTCGGTGCCAC | | |
| | TTTAAGAGCTATGCTGGA | | | |
| | AACAGCA | | | |
| Utf1_SE2_sgRNA1.fwd | ggagaaCCACCTTGTTGgAC | ctagtaCTCGAGAAAAAAAGC | 77, 78 | |
| | CTGCCTCAGTCTTCAAAC | ACCGACTCGGTGCCAC | | |
| | GTTTAAGAGCTATGCTGG | | | |
| | AAACAGCA | | | |
| Utf1_SE2_sgRNA2.fwd | ggagaaCCACCTTGTTGgAG | ctagtaCTCGAGAAAAAAAGC | 79, 80 | |
| | ACACTGAATTGACTGTGT | ACCGACTCGGTGCCAC | | |
| | GTTTAAGAGCTATGCTGG | | | |
| | AAACAGCA | | | |
| Utf1_SE2_sgRNA3.fwd | ggagaaCCACCTTGTTGGGT | ctagtaCTCGAGAAAAAAAGC | 81, 82 | |
| | CTACAGAATGAGTTCTAG | ACCGACTCGGTGCCAC | | |
| | TTTAAGAGCTATGCTGGA | | | |
| | AACAGCA | | | |
| Utf1_SE3_sgRNA1.fwd | ggagaaCCACCTTGTTGGAA | ctagtaCTCGAGAAAAAAAGC | 83, 84 | |
| | GGCATAGAGCTTTGTACG | ACCGACTCGGTGCCAC | | |
| | TTTAAGAGCTATGCTGGA | | | |
| | AACAGCA | | | |
| Utf1_SE3_sgRNA2.fwd | ggagaaCCACCTTGTTGgAC | ctagtaCTCGAGAAAAAAAGC | 85, 86 | |
| | AAGGGTCGCTCGCCCTGC | ACCGACTCGGTGCCAC | | |
| | GTTTAAGAGCTATGCTGG | | | |
| | AAACAGCA | | | |
| Utf1_SE3_sgRNA3.fwd | ggagaaCCACCTTGTTGGTT | ctagtaCTCGAGAAAAAAAGC | 87, 88 | |
| | TAGTCCACCGCTAGCTAG | ACCGACTCGGTGCCAC | | |
| | TTTAAGAGCTATGCTGGA | | | |
| | AACAGCA | | | |
| Utf1_SE3_sgRNA4.fwd | ggagaaCCACCTTGTTGgTA | ctagtaCTCGAGAAAAAAAGC | 89, 90 | |
| | GCACTAGAACCTAACCTC | ACCGACTCGGTGCCAC | | |
| | GTTTAAGAGCTATGCTGG | | | |
| | AAACAGCA | | | |
| Oct4_SE1_sgRNA1.fwd | ggagaaCCACCTTGTTGgAC | ctagtaCTCGAGAAAAAAAGC | 91, 92 | |
| | TCACAGTAAGAAAGCTGT | ACCGACTCGGTGCCAC | | |
| | GTTTAAGAGCTATGCTGG | | | |
| | AAACAGCA | | | |
| Oct4_SE1_sgRNA2.fwd | ggagaaCCACCTTGTTGgAT | ctagtaCTCGAGAAAAAAAGC | 93, 94 | |
| | ATTGGGTGGTTTACAGCT | ACCGACTCGGTGCCAC | | |
| | GTTTAAGAGCTATGCTGG | | | |
| | AAACAGCA | | | |
| Oct4_SE1_sgRNA3.fwd | ggagaaCCACCTTGTTGGTG | ctagtaCTCGAGAAAAAAAGC | 95, 96 | |
| | GGCTTCTCTGCTGTCTTGT | ACCGACTCGGTGCCAC | | |
| | TTAAGAGCTATGCTGGAA | | | |
| | ACAGCA | | | |
| Oct4_SE2_sgRNA1.fwd | ggagaaCCACCTTGTTGgCA | ctagtaCTCGAGAAAAAAAGC | 97, 98 | |
| | GGCTCACAGCTCGGGACC | ACCGACTCGGTGCCAC | | |
| | GTTTAAGAGCTATGCTGG | | | |
| | AAACAGCA | | | |
| Oct4_SE2_sgRNA2.fwd | ggagaaCCACCTTGTTGGAG | ctagtaCTCGAGAAAAAAAGC | 99, 100 | |
| | TGCTGTCTAGGCCTTAGG | ACCGACTCGGTGCCAC | | |
| | TTTAAGAGCTATGCTGGA | | | |
| | AACAGCA | | | |
| Oct4_SE2_sgRNA3.fwd | ggagaaCCACCTTGTTGGAA | ctagtaCTCGAGAAAAAAAGC | 101, 102 | |
| | CAGTGCCATAGGTTAGTG | ACCGACTCGGTGCCAC | | |
| | TTTAAGAGCTATGCTGGA | | | |
| | AACAGCA | | | |
| Oct4_SE3_sgRNA1.fwd | ggagaaCCACCTTGTTGgAA | ctagtaCTCGAGAAAAAAAGC | 103, 104 | |
| | ACCACTCTAGGGAAGTTC | ACCGACTCGGTGCCAC | | |
| | GTTTAAGAGCTATGCTGG | | | |
| | AAACAGCA | | | |
| Oct4_SE3_sgRNA2.fwd | ggagaaCCACCTTGTTGGGG | ctagtaCTCGAGAAAAAAAGC | 105, 106 | |
| | TGGAGAAACCCAACGGG | ACCGACTCGGTGCCAC | | |
| | GTTTAAGAGCTATGCTGG | | | |
| | AAACAGCA | | | |
| Oct4_SE3_sgRNA3.fwd | ggagaaCCACCTTGTTGgCC | ctagtaCTCGAGAAAAAAAGC | 107, 108 | |
| | CCCACCAGGTGGGGGTG | ACCGACTCGGTGCCAC | | |
| | AGTTTAAGAGCTATGCTG | | | |
| | GAAACAGCA | | | |
| Sox2_SE1_sgRNA1.fwd | ggagaaCCACCTTGTTGgCA | ctagtaCTCGAGAAAAAAAGC | 109, 110 | |
| | GTGTACCTTGTATCCATA | ACCGACTCGGTGCCAC | | |
| | GTTTAAGAGCTATGCTGG | | | |
| | AAACAGCA | | | |
| Sox2_SE1_sgRNA2.fwd | ggagaaCCACCTTGTTGgTC | ctagtaCTCGAGAAAAAAAGC | 111, 112 | |
| | CTCGGAATGGTTGGCGAG | ACCGACTCGGTGCCAC | | |
| | GTTTAAGAGCTATGCTGG | | | |
| | AAACAGCA | | | |
| Sox2_SE1_sgRNA3.fwd | ggagaaCCACCTTGTTGgTG | ctagtaCTCGAGAAAAAAAGC | 113, 114 | |
| | CTTGGCAGTTAAGGCTTC | ACCGACTCGGTGCCAC | | |
| | GTTTAAGAGCTATGCTGG | | | |
| | AAACAGCA | | | |
| Sox2_SE1_sgRNA4.fwd | ggagaaCCACCTTGTTGgTT | ctagtaCTCGAGAAAAAAAGC | 115, 116 | |
| | AGGGGACTATGATGGTGT | ACCGACTCGGTGCCAC | | |
| | GTTTAAGAGCTATGCTGG | | | |
| | AAACAGCA | | | |
| Sox2_SE2_sgRNA1.fwd | ggagaaCCACCTTGTTGGTA | ctagtaCTCGAGAAAAAAAGC | 117, 118 | |
| | AAAGCAAGTCCACCAGC | ACCGACTCGGTGCCAC | | |
| | GTTTAAGAGCTATGCTGG | | | |
| | AAACAGCA | | | |
| Sox2_SE2_sgRNA2.fwd | ggagaaCCACCTTGTTGgCA | ctagtaCTCGAGAAAAAAAGC | 119, 120 | |
| | ATTTTTCTGGGTCTAAAG | ACCGACTCGGTGCCAC | | |
| | GTTTAAGAGCTATGCTGG | | | |
| | AAACAGCA | | | |
| Sox2_SE2_sgRNA3.fwd | ggagaaCCACCTTGTTGgAA | ctagtaCTCGAGAAAAAAAGC | 121, 122 | |
| | TGCACTTGGGTACAAAAG | ACCGACTCGGTGCCAC | | |
| | GTTTAAGAGCTATGCTGG | | | |
| | AAACAGCA | | | |
| Sox2_SE2_sgRNA4.fwd | ggagaaCCACCTTGTTGgCG | ctagtaCTCGAGAAAAAAAGC | 123, 124 | |
| | GACGTGGGGCTGTGGCTC | ACCGACTCGGTGCCAC | | |
| | GTTTAAGAGCTATGCTGG | | | |
| | AAACAGCA | | | |
| Sox2_SE3_sgRNA1.fwd | ggagaaCCACCTTGTTGgAA | ctagtaCTCGAGAAAAAAAGC | 125, 126 | |
| | CTGGCGGCGGCCGGTACT | ACCGACTCGGTGCCAC | | |
| | GTTTAAGAGCTATGCTGG | | | |
| | AAACAGCA | | | |
| Sox2_SE3_sgRNA2.fwd | ggagaaCCACCTTGTTGgTC | ctagtaCTCGAGAAAAAAAGC | 127, 128 | |
| | GTTTTTAGGGTAAGGTAC | ACCGACTCGGTGCCAC | | |
| | GTTTAAGAGCTATGCTGG | | | |
| | AAACAGCA | | | |
| Sox2_SE3_sgRNA3.fwd | ggagaaCCACCTTGTTGGAC | ctagtaCTCGAGAAAAAAAGC | 129, 130 | |
| | TCAGCCTCTCAACTTAAG | ACCGACTCGGTGCCAC | | |
| | TTTAAGAGCTATGCTGGA | | | |
| | AACAGCA | | | |
| Sox2_SE3_sgRNA4.fwd | ggagaaCCACCTTGTTGgTA | ctagtaCTCGAGAAAAAAAGC | 131, 132 | |
| | GTTGCTGAAATAGGGAA | ACCGACTCGGTGCCAC | | |
| | GGTTTAAGAGCTATGCTG | | | |
| | GAAACAGCA | | | |
| UpE1_deletion_L_sgRNA1 | CACCGTTTGGGACATGCG | AAACGTGCATCCGCATGTC | 133, 134 | Primers used |
| | GATGCAC | CCAAAC | | to clone |
| UpE1_deletion_L_sgRNA2 | CACCGCCTCCATCTGGTC | AAACGATAATGGACCAGAT | 135, 136 | sgRNA into |
| | CATTATC | GGAGGC | | pX458 for |
| UpE1_deletion_R_sgRNA1 | CACCGGTGTTCCATTGGT | AAACCTCTAAGACCAATGG | 137, 138 | enhancer |
| | CTTAGAG | AACACC | | deletion |
| UpE1_deletion_R_sgRNA2 | CACCGGTCACTCTCAAGT | AAACATGGAACACTTGAGA | 139, 140 | |
| | GTTCCAT | GTGACC | | |
| UpE2_deletion_L_sgRNA1 | CACCGTAGAAAATCAGTA | AAACGAGTCCCTACTGATT | 141, 142 | |
| | GGGACTC | TTCTAC | | |
| UpE2_deletion_L_sgRNA2 | CACCGAAGTTATTATTAC | AAACCTAACTAGTAATAAT | 143, 144 | |
| | TAGTTAG | AACTTC | | |
| UpE2_deletion_R_sgRNA1 | CACCGGAAGGATTTAAAG | AAACGTGCAGACTTTAAAT | 145, 146 | |
| | TCTGCAC | CCTTCC | | |
| UpE2_deletion_R_sgRNA2 | CACCGATGGTACACATTT | AAACACCATCAAAATGTGT | 147, 148 | |
| | TGATGGT | ACCATC | | |
| UpE3_deletion_L_sgRNA1 | CACCGTAATATATATTCC | AAACTGATGACGGAATATA | 149, 150 | |
| | GTCATCA | TATTAC | | |
| UpE3_deletion_L_sgRNA2 | CACCGTATCAATACTGTT | AAACTTGTGAGAACAGTAT | 151, 152 | |
| | CTCACAA | TGATAC | | |
| UpE3_deletion_R_sgRNA1 | CACCGCTGTTAACTTACT | AAACGACAAATAGTAAGTT | 153, 154 | |
| | ATTTGTC | AACAGC | | |
| UpE3_deletion_R_sgRNA2 | CACCGTGCCCCAAAGTCA | AAACGATATGGTGACTTTG | 155, 156 | |
| | CCATATC | GGGCAC | | |
| HBD-1K_L_sgRNA1 | CACCGCCAGAACCTATTT | AAACGTTATTGAAATAGGT | 157, 158 | |
| | CAATAAC | TCTGGC | | |
| HBD-1K_L_sgRNA2 | CACCGCCAACCTCTCAAA | AAACAGGGAATTTTGAGAG | 159, 160 | |
| | ATTCCCT | GTTGGC | | |
| HBD-1K_R_sgRNA1 | CACCGTCGAACTGTTGAT | AAACACCTCTAATCAACAG | 161, 162 | |
| | TAGAGGT | TTCGAC | | |
| HBD-1K_R_sgRNA2 | CACCGGGAAACAATGAGG | AAACGTCAGGTCCTCATTG | 163, 164 | |
| | ACCTGAC | TTTCCC | | |
| HBD-1K_L_sgRNA3 | CACCGAGTGTTTTAGGCT | AAACCTATATTAGCCTAAA | 165, 166 | |
| | AATATAG | ACACTC | | |
| HBD-1K_R_sgRNA3 | CACCGAAGAGTGGTGATT | AAACATCTATTAATCACCA | 167, 168 | |
| | AATAGAT | CTCTTC | | |
| DnE3_L_sgRNA1 | CACCGGTAGGTCAGTTTT | AAACTCTGATTAAAACTGA | 169, 170 | |
| | AATCAGA | CCTACC | | |
| DnE3_L_sgRNA2 | CACCGTATCCCCTCTGAG | AAACGACAGTGCTCAGAGG | 171, 172 | |
| | CACTGTC | GGATAC | | |
| DnE3_R_sgRNA1 | CACCGTCACCACAAAAAA | AAACTCCAACTTTTTTTGT | 173, 174 | |
| | AGTTGGA | GGTGAC | | |
| DnE3_R_sgRNA2 | CACCGATATGCACTTATT | AAACGGCAAATAATAAGTG | 175, 176 | |
| | ATTTGCC | CATATC | | |
| DnE2_R_sgRNA1 | CACCGACTATTCTTATTC | AAACCACAGTGGAATAAGA | 177, 178 | |
| | CACTGTG | ATAGTC | | |
| DnE3_R_sgRNA2 | CACCGAAGGCTTTACTAA | AAACTATCAAATTAGTAAA | 179, 180 | |
| | TTTGATA | GCCTTC | | |
| DnE1_R_sgRNA1 | CACCGCTCGTCAGGATAT | AAACAGCAATAATATCCTG | 181, 182 | |
| | TATTGCT | ACGAGC | | |
| DnE1_R_sgRNA2 | CACCGAAAAGAGTAGAC | AAACGTGGGGATGTCTACT | 183, 184 | |
| | ATCCCCAC | CTTTTC | | |
| UpE1_deletion_wt | AGCTGGGTGTGGTGGTGA | TCAACTTTGCTATCCTCTTA | 185, 186 | Genotyping |
| | GCGCC | CATCTGTGCCTGCT | | primer for |
| UpE1_deletion_del | TGGCAGAACTTATCTACC | AGACGAAAAGGTTTGGTGGT | 187, 188 | deletion |
| | GCCACAGGAGT | GGCTCAAGG | | |
| UpE2_deletion_wt | TGAGGATATACAAGGGCA | AGGGTACCTCTGCCTCTGGT | 189, 190 | |
| | CTGA | | | |
| UpE2_deletion_del | AGGGTGGTTGGGCCACCT | GGTGAGGGCCAGGGAAGGCC | 191, 192 | |
| | AGAGACA | CC | | |
| UpE3_deletion_wt | TGCTTCTTACAGGCAGAT | ACCTTCCACTGTGCTCCCAC | 193, 194 | |
| | TTCCTTGGGCATCA | TGCCT | | |
| UpE3_deletion_del | TGGTGACGAGGGTACCTC | GGGCAAAGCTCTACATTAGG | 195, 196 | |
| | CAAGGCA | CATTTTGAGGAGG | | |
| DnE3_wt | ACATTCCTATTTGCCAAG | AGACTCTTGAGGGCCTGACC | 197, 198 | |
| | GCAGTGGAGTTTTTGC | TCGCT | | |
| DnE3_del | AGGTGTGCCAGATGCTCC | GGGATGGGAAGGGAAAGA | 199, 200 | |
| | ACCTGT | AGTTGATCTTCAGTTAG | | |
| DnE2_wt | CTGTGTTCACTATGCAGT | CTAGCAGCCTAGGTATGGG | 201, 202 | |
| | GTGAGAG | TACTCG | | |
| DnE1_wt | CCAGACAACTGGTTAAGA | AGCATTACTGTTCACACAA | 203, 204 | |
| | GAGAGG | GGCAC | | |
| DnE1/2_del | CTATAAGAAACTGGTAAA | AAATCTAGGGTCGAAAGCC | 205, 206 | |
| | CACTGAATG | ACAGC | | |
| HBD-1K_wt | ATCAAGCATCCAGCATTT | GAAACGAAGAGAGGGGAA | 207, 208 | |
| | GT | GG | | |
| HBD-1K_del | TCCCTTAACTTGCCCTGA | AGGCACCTCAGACTCAGCA | 209, 210 | |
| | GA | T | | |
| Telomere-PCR | GGTTTTTGAGGGTGAGGG | TCCCGACTATCCCTATCCCT | 211, 212 | qPCR primers |
| | TGAGGGTGAGGGTGAGG | ATCCCTATCCCTATCCCTA | | for Telomere |
| | GT | | | |
| human 36B4 | CAGCAAGTGGGAAGGTG | CCCATTCTATCATCAACGG | 213, 214 | qPCR primers |
| | TAATCC | GTACAA | | for 36B4 |
| hHBE1_RT | GCAAGAAGGTGCTGACTT | ACCATCACGTTACCCAGGA | 215, 216 | qRT-PCR |
| | CC | G | | primer |
| hHBG_RT | TGGATGATCTCAAGGGCA | TCAGTGGTATCTGGAGGAC | 217, 218 | |
| | C | A | | |
| hHBB_RT | CTGAGGAGAAGTCTGCCG | AGCATCAGGAGTGGACAGA | 219, 220 | |
| | TTA | T | | |
| hGATA1_RT | CATGCGGAAGGATGGTAT | CTCCCCACAATTCCCGCTAC | 221, 222 | |
| | TCAG | | | |
| hGATA2_RT | GCAACCCCTACTATGCCA | CAGTGGCGTCTTGGAGAAG | 223, 224 | |
| | ACC | | | |
| hGAPDH_RT | ACCCAGAAGACTGTGGAT | TTCAGCTCAGGGATGACCTT | 225, 226 | |
| | GG | | | |
| mGapdh_RT | TGGTGAAGGTCGGTGTGA | CCATGTAGTTGAGGTCAAT | 227, 228 | |
| | AC | GAAGG | | |
| mOct4_RT | CTCCCGAGGAGTCCCAGG | GATGGTGGTCTGGCTGAAC | 229, 230 | |
| | ACAT | ACCT | | |
| mSox2_RT | AAGAAAGGAGAGAAGTT | GAGATCTGGCGGAGAATAG | 231, 232 | |
| | TGGAGCC | TTGG | | |
| mUtf1_RT | GGAAGAACTGAATCTGA | CTCTACTGGCCCTGGACG | 233, 234 | |
| | GCG | | | |
| mEsrrb_RT | ATGAAGGAGCCGCAACT | GAGGAGCCAAGCAACGAGT | 235, 236 | |
| | AGA | | | |
| Vimentin_RT | CGGCTGCGAGAGAAATT | CCACTTTCCGTTCAAGGTCA | 237, 238 | |
| | GC | AG | | |
| Gata4_RT | CACAAGATGAACGGCAT | CAGCGTGGTGGTGGTAGTC | 239, 240 | |
| | CAACC | TG | | |
| Gata6_RT | GGTCTCTACAGCAAGATG | TGGCACAGGACAGTCCAAG | 241, 242 | |
| | AATGG | | | |
| pGIPZ-ARID1A- | TGCTGTTGACAGTGAGCG | | 243 | Lentiviral |
| sh1_RHS4430-98818306 | CCCGCAGGAGCTATCTCA | | | shRNA in the |
| | AGATTAGTGAAGCCACA | | | pGIPZ vector |
| | GATGTAATCTTGAGATAG | | | |
| | CTCCTGCGGTTGCCTACT | | | |
| | GCCTCGGA | | | |
| pGIPZ-ARID1A- | TGCTGTTGACAGTGAGCG | | 244 | |
| sh2_RHS4430-98894847 | AGCATGTCCTATGAGCCA | | | |
| | AATATAGTGAAGCCACA | | | |
| | GATGTATATTTGGCTCAT | | | |
| | AGGACATGCGTGCCTACT | | | |
| | GCCTCGGA | | | |
| pGIPZ-ARID1B- | TGCTGTTGACAGTGAGCG | | 245 | |
| sh1_RHS4430-98715739 | ACGAAAGATTACCTCCAA | | | |
| | AGATTAGTGAAGCCACA | | | |
| | GATGTAATCTTTGGAGGT | | | |
| | AATCTTTCGCTGCCTACT | | | |
| | GCCTCGGA | | | |
| pGIPZ-ARID1B- | TGCTGTTGACAGTGAGCG | | 246 | |
| sh2_RHS4430-99157258 | CCCTCATTTCATGGAGAT | | | |
| | GAAATAGTGAAGCCACA | | | |
| | GATGTATTTCATCTCCAT | | | |
| | GAAATGAGGATGCCTACT | | | |
| | GCCTCGGA | | | |
| pGIPZ-ARID1B- | TGCTGTTGACAGTGAGCG | | 247 | |
| sh3_RHS4430-99161431 | CGGGCTTTGGACACTATT | | | |
| | AATATAGTGAAGCCACA | | | |
| | GATGTATATTAATAGTGT | | | |
| | CCAAAGCCCATGCCTACT | | | |
| | GCCTCGGA | | | |
| pGIPZ-EP400- | TGCTGTTGACAGTGAGCG | | 248 | |
| sh1_RHS4430-99151538 | ACCGTACTGGCAGGAACC | | | |
| | ATTATAGTGAAGCCACAG | | | |
| | ATGTATAATGGTTCCTGC | | | |
| | CAGTACGGCTGCCTACTG | | | |
| | CCTCGGA | | | |
| pGIPZ-EP400- | TGCTGTTGACAGTGAGCG | | 249 | |
| sh2_RHS4430-99167161 | ACCAGTCTCCCAGTTATC | | | |
| | AAATTAGTGAAGCCACA | | | |
| | GATGTAATTTGATAACTG | | | |
| | GGAGACTGGGTGCCTACT | | | |
| | GCCTCGGA | | | |
| pGIPZ-MATR3- | TGCTGTTGACAGTGAGCG | | 250 | |
| sh1_RHS4430-98910514 | CGGTTATTATGACAGAAT | | | |
| | GGATTAGTGAAGCCACA | | | |
| | GATGTAATCCATTCTGTC | | | |
| | ATAATAACCATGCCTACT | | | |
| | GCCTCGGA | | | |
| pGIPZ-MATR3- | TGCTGTTGACAGTGAGCG | | 251 | |
| sh2_RHS4430-98913492 | CGGTTGACCTGTCTGAGA | | | |
| | AATATAGTGAAGCCACA | | | |
| | GATGTATATTTCTCAGAC | | | |
| | AGGTCAACCTTGCCTACT | | | |
| | GCCTCGGA | | | |
| pGIPZ-NUP153- | TGCTGTTGACAGTGAGCG | | 252 | |
| sh1_RHS4430-98843172 | CGCTGTTAGACGCAGGAA | | | |
| | ATAATAGTGAAGCCACA | | | |
| | GATGTATTATTTCCTGCG | | | |
| | TCTAACAGCATGCCTACT | | | |
| | GCCTCGGA | | | |
| pGIPZ-NUP153- | TGCTGTTGACAGTGAGCG | | 253 | |
| sh2_RHS4430-99151347 | CCCTTAGGATTTGGAGAT | | | |
| | AAATTAGTGAAGCCACA | | | |
| | GATGTAATTTATCTCCAA | | | |
| | ATCCTAAGGTTGCCTACT | | | |
| | GCCTCGGA | | | |
| pGIPZ-NUP153- | TGCTGTTGACAGTGAGCG | | 254 | |
| sh3_RHS4430-99158692 | ACGCAACAAGCCCAGTA | | | |
| | GTTTATAGTGAAGCCACA | | | |
| | GATGTATAAACTACTGGG | | | |
| | CTTGTTGCGGTGCCTACT | | | |
| | GCCTCGGA | | | |
| pGIPZ-NUP214- | TGCTGTTGACAGTGAGCG | | 255 | |
| sh1_RHS4430-98704462 | AGCTTGCTAGTTCCTATG | | | |
| | AAATTAGTGAAGCCACA | | | |
| | GATGTAATTTCATAGGAA | | | |
| | CTAGCAAGCCTGCCTACT | | | |
| | GCCTCGGA | | | |
| pGIPZ-NUP214- | TGCTGTTGACAGTGAGCG | | 256 | |
| sh2_RHS4430-99150987 | CCCATAGAATCTCACACC | | | |
| | AAATTAGTGAAGCCACA | | | |
| | GATGTAATTTGGTGTGAG | | | |
| | ATTCTATGGTTGCCTACT | | | |
| | GCCTCGGA | | | |
| pGIPZ-NUP54- | TGCTGTTGACAGTGAGCG | | 257 | |
| sh1_RHS4430-98818214 | ACCAGTCCAACCAGCTGA | | | |
| | TAAATAGTGAAGCCACA | | | |
| | GATGTATTTATCAGCTGG | | | |
| | TTGGACTGGGTGCCTACT | | | |
| | GCCTCGGA | | | |
| pGIPZ-NUP98- | TGCTGTTGACAGTGAGCG | | 258 | |
| sh1_RHS4430-99139612 | CCCTGTTAATCGTGATTC | | | |
| | AGAATAGTGAAGCCACA | | | |
| | GATGTATTCTGAATCACG | | | |
| | ATTAACAGGATGCCTACT | | | |
| | GCCTCGGA | | | |
| pGIPZ-NUP98- | TGCTGTTGACAGTGAGCG | | 259 | |
| sh2_RHS4430-98709406 | CCCTCTCCCATCCTCCTC | | | |
| | GAAATAGTGAAGCCACA | | | |
| | GATGTATTTCGAGGAGGA | | | |
| | TGGGAGAGGTTGCCTACT | | | |
| | GCCTCGGA | | | |
| pGIPZ-SMC2- | TGCTGTTGACAGTGAGCG | | 260 | |
| sh1_RHS4430-98901433 | ACCAGATTTACTCAATGT | | | |
| | CAAATAGTGAAGCCACA | | | |
| | GATGTATTTGACATTGAG | | | |
| | TAAATCTGGCTGCCTACT | | | |
| | GCCTCGGA | | | |
| pGIPZ-SMC3- | TGCTGTTGACAGTGAGCG | | 261 | |
| sh1_RHS4430-98715413 | CGCAGTGCAACACAGAA | | | |
| | TTAAATAGTGAAGCCACA | | | |
| | GATGTATTTAATTCTGTG | | | |
| | TTGCACTGCTTGCCTACT | | | |
| | GCCTCGGA | | | |
| pGIPZ-SMC3- | TGCTGTTGACAGTGAGCG | | 262 | |
| sh2_RHS4430-98843956 | AGCAGAAATATTGAAAG | | | |
| | GATTATAGTGAAGCCACA | | | |
| | GATGTATAATCCTTTCAA | | | |
| | TATTTCTGCGTGCCTACT | | | |
| | GCCTCGGA | | | |
| pGIPZ-SMC3- | TGCTGTTGACAGTGAGCG | | 263 | |
| sh3_RHS4430-98902085 | CCCACACATGGTTAATTG | | | |
| | GAAATAGTGAAGCCACA | | | |
| | GATGTATTTCCAATTAAC | | | |
| | CATGTGTGGTTGCCTACT | | | |
| | GCCTCGGA | | | |
| pGIPZ-SMC3- | TGCTGTTGACAGTGAGCG | | 264 | |
| sh4_RHS4430-99168129 | CGGGCAGAAATGGATCT | | | |
| | GGAAATAGTGAAGCCAC | | | |
| | AGATGTATTTCCAGATCC | | | |
| | ATTTCTGCCCATGCCTAC | | | |
| | TGCCTCGGA | | | |
| pGIPZ-STAT1- | TGCTGTTGACAGTGAGCG | | 265 | |
| sh1_RHS4430-98484782 | CGGCCCTAAAGGAACTG | | | |
| | GATATTAGTGAAGCCACA | | | |
| | GATGTAATATCCAGTTCC | | | |
| | TTTAGGGCCATGCCTACT | | | |
| | GCCTCGGA | | | |
| pGIPZ-STAT1- | TGCTGTTGACAGTGAGCG | | 266 | |
| sh2_RHS4430-98521615 | ACCTGAAGTATCTGTATC | | | |
| | CAAATAGTGAAGCCACA | | | |
| | GATGTATTTGGATACAGA | | | |
| | TACTTCAGGGTGCCTACT | | | |
| | GCCTCGGA | | | |
| pGIPZ-STAT1- | TGCTGTTGACAGTGAGCG | | 267 | |
| sh3_RHS4430-98818169 | CGCAAGCGTAATCTTCAG | | | |
| | GATATAGTGAAGCCACA | | | |
| | GATGTATATCCTGAAGAT | | | |
| | TACGCTTGCTTGCCTACT | | | |
| | GCCTCGGA | | | |
| pGIPZ-STAT1- | TGCTGTTGACAGTGAGCG | | 268 | |
| sh4_RHS4430-98901335 | CCAGCTGTTACTCAAGAA | | | |
| | GATGTAGTGAAGCCACA | | | |
| | GATGTACATCTTCTTGAG | | | |
| | TAACAGCTGTTGCCTACT | | | |
| | GCCTCGGA | | | |
| pGIPZ-TBL1XR1- | TGCTGTTGACAGTGAGCG | | 269 | |
| sh1_RHS4430-98526016 | AGGCAGCATAAAGGCCC | | | |
| | TATATTAGTGAAGCCACA | | | |
| | GATGTAATATAGGGCCTT | | | |
| | TATGCTGCCCTGCCTACT | | | |
| | GCCTCGGA | | | |
| pGIPZ-TBL1XR1- | TGCTGTTGACAGTGAGCG | | 270 | |
| sh2_RHS4430-98893812 | CGGAGCACATACTATAGC | | | |
| | AAATTAGTGAAGCCACA | | | |
| | GATGTAATTTGCTATAGT | | | |
| | ATGTGCTCCATGCCTACT | | | |
| | GCCTCGGA | | | |
| pGIPZ-TBL1XR1- | TGCTGTTGACAGTGAGCG | | 271 | |
| sh3_RHS4430-99148532 | CCCATGATTTGCAAGCAC | | | |
| | ATAATAGTGAAGCCACA | | | |
| | GATGTATTATGTGCTTGC | | | |
| | AAATCATGGATGCCTACT | | | |
| | GCCTCGGA | | | |
| shNT | CCGGCAACAAGATGAAG | | 272 | Lentiviral |
| | AGCACCAACTCGAGTTGG | | | shRNA in the |
| | TGCTCTTCATCTTGTTGTT | | | pLKO vector |
| | TTT | | | |
| BCL11A- | CCGGCGCACAGAACACTC | | 273 | |
| sh49_TRCN0000033449 | ATGGATTCTCGAGAATCC | | | |
| | ATGAGTGTTCTGTGCGTT | | | |
| | TTTG | | | |
| BCL11A- | CCGGCCAGAGGATGACG | | 274 | |
| sh51_TRCN0000033451 | ATTGTTTACTCGAGTAAA | | | |
| | CAATCGTCATCCTCTGGT | | | |
| | TTTTG | | | |
| BCL11A- | CCGGGCATAGACGATGG | | 275 | |
| sh53_TRCN0000033453 | CACTGTTACTCGAGTAAC | | | |
| | AGTGCCATCGTCTATGCT | | | |
| | TTTTG | | | |
| CHD4- | CCGGGCTGACACAGTTAT | | 276 | |
| sh4_TRCN0000021362 | TATCTATCTCGAGATAGA | | | |
| | TAATAACTGTGTCAGCTT | | | |
| | TTT | | | |
| CHD4- | CCGGGCGGGAGTTCAGTA | | 277 | |
| sh5_TRCN0000021363 | CCAATAACTCGAGTTATT | | | |
| | GGTACTGAACTCCCGCTT | | | |
| | TTT | | | |
| EED- | CCGGGCAAACTTTATGTT | | 278 | |
| sh1_TRCN0000021204 | TGGGATTCTCGAGAATCC | | | |
| | CAAACATAAAGTTTGCTT | | | |
| | TTT | | | |
| EED- | CCGGCCAGAGACATACAT | | 279 | |
| sh2_TRCN0000021205 | AGGAATTCTCGAGAATTC | | | |
| | CTATGTATGTCTCTGGTTT | | | |
| | TT | | | |
| EED- | CCGGGCAGCATTCTTATA | | 280 | |
| sh3_TRCN0000021206 | GCTGTTTCTCGAGAAACA | | | |
| | GCTATAAGAATGCTGCTT | | | |
| | TTT | | | |
| EED- | CCGGCCTATAACAATGCA | | 281 | |
| sh4_TRCN0000021207 | GTGTATACTCGAGTATAC | | | |
| | ACTGCATTGTTATAGGTT | | | |
| | TTT | | | |
| EED- | CCGGCCAGTGAATCTAAT | | 282 | |
| sh5_TRCN0000021208 | GTGACTACTCGAGTAGTC | | | |
| | ACATTAGATTCACTGGTT | | | |
| | TTT | | | |
| HDAC1- | CCGGCGTTCTTAACTTTG | | 283 | |
| sh2_TRCN0000004814 | AACCATACTCGAGTATGG | | | |
| | TTCAAAGTTAAGAACGTT | | | |
| | TTT | | | |
| HDAC1- | CCGGGCCGGTCATGTCCA | | 284 | |
| sh3_TRCN0000004816 | AAGTAATCTCGAGATTAC | | | |
| | TTTGGACATGACCGGCTT | | | |
| | TTT | | | |
| HDAC1- | CCGGGCTGCTCAACTATG | | 285 | |
| sh5_TRCN0000004818 | GTCTCTACTCGAGTAGAG | | | |
| | ACCATAGTTGAGCAGCTT | | | |
| | TTT | | | |
| HDAC2- | CCGGGCAGACTCATTATC | | 286 | |
| sh1_TRCN0000004822 | TGGTGATCTCGAGATCAC | | | |
| | CAGATAATGAGTCTGCTT | | | |
| | TTT | | | |
| HDAC2- | CCGGGCAAATACTATGCT | | 287 | |
| sh2_TRCN0000004823 | GTCAATTCTCGAGAATTG | | | |
| | ACAGCATAGTATTTGCTT | | | |
| | TTT | | | |
| HDAC2- | CCGGCAGTCTCACCAATT | | 288 | |
| sh3_TRCN0000004819 | TCAGAAACTCGAGTTTCT | | | |
| | GAAATTGGTGAGACTGTT | | | |
| | TTT | | | |
| HDAC2- | CCGGCCAGCGTTTGATGG | | 289 | |
| sh4_TRCN0000004820 | ACTCTTTCTCGAGAAAGA | | | |
| | GTCCATCAAACGCTGGTT | | | |
| | TTT | | | |
| IKZF1- | CCGGGCGGAGGATTTACG | | 290 | |
| sh2_TRCN0000107871 | AATGCTTCTCGAGAAGCA | | | |
| | TTCGTAAATCCTCCGCTT | | | |
| | TTTG | | | |
| IKZF1- | CCGGCCGTTGGTAAACCT | | 291 | |
| sh3_TRCN0000107872 | CACAAATCTCGAGATTTG | | | |
| | TGAGGTTTACCAACGGTT | | | |
| | TTTG | | | |
| IKZF1- | CCGGGCCGAAGCTATAA | | 292 | |
| sh4_TRCN0000107873 | ACAGCGAACTCGAGTTCG | | | |
| | CTGTTTATAGCTTCGGCT | | | |
| | TTTTG | | | |
| IKZF1- | CCGGCGCCAAACGTAAG | | 293 | |
| sh5_TRCN0000107874 | AGCTCTATCTCGAGATAG | | | |
| | AGCTCTTACGTTTGGCGT | | | |
| | TTTTG | | | |
| KDM3A- | CCGGCCCAAGATGTATAA | | 294 | |
| sh1_TRCN0000021149 | TGCTTATCTCGAGATAAG | | | |
| | CATTATACATCTTGGGTT | | | |
| | TTT | | | |
| KDM3A- | CCGGCCCTAATAACTGTT | | 295 | |
| sh2_TRCN0000021150 | CAGGAAACTCGAGTTTCC | | | |
| | TGAACAGTTATTAGGGTT | | | |
| | TTT | | | |
| KDM3A- | CCGGGCTGGTATTTAGAC | | 296 | |
| sh3_TRCN0000021151 | CGATCATCTCGAGATGAT | | | |
| | CGGTCTAAATACCAGCTT | | | |
| | TTT | | | |
| KDM3A- | CCGGGCTTTGATTGTGAA | | 297 | |
| sh4_TRCN0000021152 | GCATTTACTCGAGTAAAT | | | |
| | GCTTCACAATCAAAGCTT | | | |
| | TTT | | | |
| KDM3A- | CCGGCCATACGTTTAACA | | 298 | |
| sh5_TRCN0000021153 | GCACAATCTCGAGATTGT | | | |
| | GCTGTTAAACGTATGGTT | | | |
| | TTT | | | |
| KDM3B- | CCGGCCCTAGTTCATCGC | | 299 | |
| sh1_TRCN0000017093 | AACCTTTCTCGAGAAAGG | | | |
| | TTGCGATGAACTAGGGTT | | | |
| | TTT | | | |
| KDM3B- | CCGGGCGATCTTTGTAGA | | 300 | |
| sh2_TRCN0000017095 | ATTTGATCTCGAGATCAA | | | |
| | ATTCTACAAAGATCGCTT | | | |
| | TTT | | | |
| KDM3B- | CCGGGCTGTTAATGTGAT | | 301 | |
| sh3_TRCN0000017096 | GGTGTATCTCGAGATACA | | | |
| | CCATCACATTAACAGCTT | | | |
| | TTT | | | |
| KDM3B- | CCGGCCTTGTAGATAAAC | | 302 | |
| sh4_TRCN0000017097 | TGGGTTTCTCGAGAAACC | | | |
| | CAGTTTATCTACAAGGTT | | | |
| | TTT | | | |
| KLF1- | CCGGTGCACATGAAGCGC | | 303 | |
| sh1_TRCN0000230814 | CACCTTTCTCGAGAAAGG | | | |
| | TGGCGCTTCATGTGCATT | | | |
| | TTTG | | | |
| KLF1- | CCGGCCCTCCTTCCTGAG | | 304 | |
| sh4_TRCN0000230812 | TTGTTTGCTCGAGCAAAC | | | |
| | AACTCAGGAAGGAGGGT | | | |
| | TTTTG | | | |
| KLF1- | CCGGCAGAGGATCCAGG | | 305 | |
| sh5_TRCN0000230813 | TGTGATAGCTCGAGCTAT | | | |
| | CACACCTGGATCCTCTGT | | | |
| | TTTTG | | | |
| NCOR1- | CCGGCGCAGTATTGTCCA | | 306 | |
| sh3_TRCN0000060655 | AATTATTCTCGAGAATAA | | | |
| | TTTGGACAATACTGCGTT | | | |
| | TTTG | | | |
| NCOR1- | CCGGGCCATCAAACACA | | 307 | |
| sh4_TRCN0000060656 | ATGTCAAACTCGAGTTTG | | | |
| | ACATTGTGTTTGATGGCT | | | |
| | TTTTG | | | |
| NCOR1- | CCGGGCTCTCAAAGTTCA | | 308 | |
| sh5_TRCN0000060657 | GACTCTTCTCGAGAAGAG | | | |
| | TCTGAACTTTGAGAGCTT | | | |
| | TTTG | | | |
| NCOR2- | CCGGCCTCTATTACTACC | | 309 | |
| sh2_TRCN0000060704 | TGACTAACTCGAGTTAGT | | | |
| | CAGGTAGTAATAGAGGTT | | | |
| | TTTG | | | |
| NCOR2- | CCGGGCAGTGTAAGAACT | | 310 | |
| sh5_TRCN0000060707 | TCTACTTCTCGAGAAGTA | | | |
| | GAAGTTCTTACACTGCTT | | | |
| | TTTG | | | |
| RBBP4- | CCGGCCCTTGTATCATCG | | 311 | |
| sh2_TRCN0000115869 | CAACAAACTCGAGTTTGT | | | |
| | TGCGATGATACAAGGGTT | | | |
| | TTTG | | | |
| RBBP4- | CCGGGCCTTTCTTTCAAT | | 312 | |
| sh3_TRCN0000115870 | CCTTATACTCGAGTATAA | | | |
| | GGATTGAAAGAAAGGCT | | | |
| | TTTTG | | | |
| RBBP4- | CCGGCGGCAGTAGTAGA | | 313 | |
| sh4_TRCN0000115868 | AGATGTTTCTCGAGAAAC | | | |
| | ATCTTCTACTACTGCCGT | | | |
| | TTTTG | | | |
| RBBP4- | CCGGGCAGACTGAATGTC | | 314 | |
| sh5_TRCN0000115871 | TGGGATTCTCGAGAATCC | | | |
| | CAGACATTCAGTCTGCTT | | | |
| | TTTG | | | |
| SMARCA4- | CCGGCCATATTTATACAG | | 315 | |
| sh1_TRCN0000015548 | CAGAGAACTCGAGTTCTC | | | |
| | TGCTGTATAAATATGGTT | | | |
| | TTT | | | |
| SMARCA4- | CCGGCCCGTGGACTTCAA | | 316 | |
| sh2_TRCN0000015549 | GAAGATACTCGAGTATCT | | | |
| | TCTTGAAGTCCACGGGTT | | | |
| | TTT | | | |
| SMARCA4- | CCGGCCGAGGTCTGATAG | | 317 | |
| sh4_TRCN0000015551 | TGAAGAACTCGAGTTCTT | | | |
| | CACTATCAGACCTCGGTT | | | |
| | TTT | | | |
| SMARCA4- | CCGGCGGCAGACACTGTG | | 318 | |
| sh5_TRCN0000015552 | ATCATTTCTCGAGAAATG | | | |
| | ATCACAGTGTCTGCCGTT | | | |
| | TTT | | | |
| SMARCC1- | CCGGGCAGGATATTAGCT | | 319 | |
| sh1_TRCN0000015628 | CCTTATACTCGAGTATAA | | | |
| | GGAGCTAATATCCTGCTT | | | |
| | TTT | | | |
| SMARCC1- | CCGGCCCACCACATTTAC | | 320 | |
| sh2_TRCN0000015629 | CCATATTCTCGAGAATAT | | | |
| | GGGTAAATGTGGTGGGTT | | | |
| | TTT | | | |
| SMARCC1- | CCGGGCTATGATACTTGG | | 321 | |
| sh3_TRCN0000015630 | GTCCATACTCGAGTATGG | | | |
| | ACCCAAGTATCATAGCTT | | | |
| | TTT | | | |
| SMARCC1- | CCGGCCTAGCTGTTTATC | | 322 | |
| sh5_TRCN0000015632 | GACGGAACTCGAGTTCCG | | | |
| | TCGATAAACAGCTAGGTT | | | |
| | TTT | | | |
| SUZ12- | CCGGGCTTACGTTTACTG | | 323 | |
| sh2_TRCN0000038725 | GTTTCTTCTCGAGAAGAA | | | |
| | ACCAGTAAACGTAAGCTT | | | |
| | TTTG | | | |
| SUZ12- | CCGGCCAAACCTCTTGCC | | 324 | |
| sh3_TRCN0000038726 | ACTAGAACTCGAGTTCTA | | | |
| | GTGGCAAGAGGTTTGGTT | | | |
| | TTTG | | | |
| SUZ12- | CCGGCGGAATCTCATAGC | | 325 | |
| sh4_TRCN0000038727 | ACCAATACTCGAGTATTG | | | |
| | GTGCTATGAGATTCCGTT | | | |
| | TTTG | | | |
| SUZ12- | CCGGGCTGACAATCAAAT | | 326 | |
| sh5_TRCN0000038728 | GAATCATCTCGAGATGAT | | | |
| | TCATTTGATTGTCAGCTTT | | | |
| | TTG | | | |
| TRIM28- | CCGGCCTGGCTCTGTTCT | | 327 | |
| sh2_TRCN0000017999 | CTGTCCTCTCGAGAGGAC | | | |
| | AGAGAACAGAGCCAGGT | | | |
| | TTTT | | | |
| TRIM28- | CCGGCTGAGACCAAACCT | | 328 | |
| sh3_TRCN0000018001 | GTGCTTACTCGAGTAAGC | | | |
| | ACAGGTTTGGTCTCAGTT | | | |
| | TTT | | | |
| ZBTB33- | CCGGCCCTTCCATGTTAG | | 329 | |
| sh1_TRCN0000017838 | CACTTTACTCGAGTAAAG | | | |
| | TGCTAACATGGAAGGGTT | | | |
| | TTT | | | |
| ZBTB33- | CCGGCGGTGAAGATACTT | | 330 | |
| sh2_TRCN0000017840 | ATGATATCTCGAGATATC | | | |
| | ATAAGTATCTTCACCGTT | | | |
| | TTT | | | |
| | MDYKDDDDK | FLAG sequence | 331 | |
| | SGLNDIFEAQKIEWH | Biotinylation | 332 | |
| | | site in dCas9 | | |

The recombinant modified nuclease-deficient Cas9 (dCas9), with the biotinylation site is provided herein, the nucleic acid is SEQ ID NO:333, and the amino acid SEQ ID NO:334.

```
GGC CTG AAC GAC ATC TTC GAG GCT CAG AAA ATC GAA TGG CAC GAA GGC GCG CCG AGC TCG
< 60
G L N D I F E A Q K I E W H E G A P S S
                        10 20 30 40 50
AGG ATC CTT GCT AGC CCC AAA AAG AAG AGG AAA GTG GAC AAG AAG TAT TCT ATC GGA CTG
< 120
R I L A S P K K K R K V D K K Y S I G L
                        70 80 90 100 110
GCC ATC GGG ACT AAT AGC GTC GGG TGG GCC GTG ATC ACT GAC GAG TAC AAG GTG CCC TCT
< 180
A I G T N S V G W A V I T D E Y K V P S
                        130 140 150 160 170
AAG AAG TTC AAG GTG CTC GGG AAC ACC GAC CGG CAT TCC ATC AAG AAA AAT CTG ATC GGA
< 240
K K F K V L G N T D R H S I K K N L I G
                        190 200 210 220 230
GCT CTC CTC TTT GAT TCA GGG GAG ACC GCT GAA GCA ACC CGC CTC AAG CGG ACT GCT AGA
< 300
A L L F D S G E T A E A T R L K R T A R
                        250 260 270 280 290
CGG CGG TAC ACC AGG AGG AAG AAC CGG ATT TGT TAC CTT CAA GAG ATA TTC TCC AAC GAA
< 360
R R Y T R R K N R I C Y L Q E I F S N E
                        310 320 330 340 350
ATG GCA AAG GTC GAC GAC AGC TTC TTC CAT AGG CTG GAA GAA TCA TTC CTC GTG GAA GAG
< 420
M A K V D D S F F H R L E E S F L V E E
                        370 380 390 400 410
GAT AAG AAG CAT GAA CGG CAT CCC ATC TTC GGT AAT ATC GTC GAC GAG GTG GCC TAT CAC
< 480
D K K H E R H P I F G N I V D E V A Y H
                        430 440 450 460 470
GAG AAA TAC CCA ACC ATC TAC CAT CTT CGC AAA AAG CTG GTG GAC TCA ACC GAC AAG GCA
< 540
E K Y P T I Y H L R K K L V D S T D K A
                        490 500 510 520 530
GAC CTC CGG CTT ATC TAC CTG GCC CTG GCC CAC ATG ATC AAG TTC AGA GGC CAC TTC CTG
< 600
D L R L I Y L A L A H M I K F R G H F L
                        550 560 570 580 590
ATC GAG GGC GAC CTC AAT CCT GAC AAT AGC GAT GTG GAT AAA CTG TTC ATC CAG CTG GTG
< 660
I E G D L N P D N S D V D K L F I Q L V
                        610 620 630 640 650
CAG ACT TAC AAC CAG CTC TTT GAA GAG AAC CCC ATC AAT GCA AGC GGA GTC GAT GCC AAG
< 720
Q T Y N Q L F E E N P I N A S G V D A K
                        670 680 690 700 710
GCC ATT CTG TCA GCC CGG CTG TCA AAG AGC CGC AGA CTT GAG AAT CTT ATC GCT CAG CTG
< 780
A I L S A R L S K S R R L E N L I A Q L
                        730 740 750 760 770
CCG GGT GAA AAG AAA AAT GGA CTG TTC GGG AAC CTG ATT GCT CTT TCA CTT GGG CTG ACT
< 840
P G E K K N G L F G N L I A L S L G L T
                        790 800 810 820 830
CCC AAT TTC AAG TCT AAT TTC GAC CTG GCA GAG GAT GCC AAG CTG CAA CTG TCC AAG GAC
< 900
P N F K S N F D L A E D A K L Q L S K D
                        850 860 870 880 890
ACC TAT GAT GAC GAT CTC GAC AAC CTC CTG GCC CAG ATC GGT GAC CAA TAC GCC GAC CTT
< 960
T Y D D D L D N L L A Q I G D Q Y A D L
                        910 920 930 940 950
TTC CTT GCT GCT AAG AAT CTT TCT GAC GCC ATC CTG CTG TCT GAC ATT CTC CGC GTG AAC
< 1020
F L A A K N L S D A I L L S D I L R V N
                        970 980 990 1000 1010
ACT GAA ATC ACC AAG GCC CCT CTT TCA GCT TCA ATG ATT AAG CGG TAT GAT GAG CAC CAC
< 1080
T E I T K A P L S A S M I K R Y D E H H
                        1030 1040 1050 1060 1070
CAG GAC CTG ACC CTG CTT AAG GCA CTC GTC CGG CAG CAG CTT CCG GAG AAG TAC AAG GAA
< 1140
Q D L T L L K A L V R Q Q L P E K Y K E
                        1090 1100 1110 1120 1130
ATC TTC TTT GAC CAG TCA AAG AAT GGA TAC GCC GGC TAC ATC GAC GGA GGT GCC TCC CAA
< 1200
I F F D Q S K N G Y A G Y I D G G A S Q
                        1150 1160 1170 1180 1190
GAG GAA TTT TAT AAG TTT ATC AAA CCT ATC CTT GAG AAG ATG GAC GGC ACC GAA GAG CTC
< 1260
E E F Y K F I K P I L E K M D G T E E L
                        1210 1220 1230 1240 1250
CTC GTG AAA CTG AAT CGG GAG GAT CTG CTG CGG AAG CAG CGC ACT TTC GAC AAT GGG AGC
< 1320
L V K L N R E D L L R K Q R T F D N G S
                        1270 1280 1290 1300 1310
ATT CCC CAC CAG ATC CAT CTT GGG GAG CTT CAC GCC ATC CTT CGG CGC CAA GAG GAC TTC
< 1380
I P H Q I H L G E L H A I L R R Q E D F
                        1330 1340 1350 1360 1370
TAC CCC TTT CTT AAG GAC AAC AGG GAG AAG ATT GAG AAA ATT CTC ACT TTC CGC ATC CCC
< 1440
Y P F L K D N R E K I E K I L T F R I P
                        1390 1400 1410 1420 1430
TAC TAC GTG GGA CCC CTC GCC AGA GGA AAT AGC CGG TTT GCT TGG ATG ACC AGA AAG TCA
< 1500
Y Y V G P L A R G N S R F A W M T R K S
                        1450 1460 1470 1480 1490
GAA GAA ACT ATC ACT CCC TGG AAC TTC GAA GAG GTG GTG GAC AAG GGA GCC AGC GCT CAG
< 1560
E E T I T P W N F E E V V D K G A S A Q
                        1510 1520 1530 1540 1550
TCA TTC ATC GAA CGG ATG ACT AAC TTC GAT AAG AAC CTC CCC AAT GAG AAG GTC CTG CCG
< 1620
S F I E R M T N F D K N L P N E K V L P
                        1570 1580 1590 1600 1610
AAA CAT TCC CTG CTC TAC GAG TAC TTT ACC GTG TAC AAC GAG CTG ACC AAG GTG AAA TAT
< 1680
K H S L L Y E Y F T V Y N E L T K V K Y
                        1630 1640 1650 1660 1670
GTC ACC GAA GGG ATG AGG AAG CCC GCA TTC CTG TCA GGC GAA CAA AAG AAG GCA ATT GTG
< 1740
V T E G M R K P A F L S G E Q K K A I V
                        1690 1700 1710 1720 1730
GAC CTT CTG TTC AAG ACC AAT AGA AAG GTG ACC GTG AAG CAG CTG AAG GAG GAC TAT TTC
< 1800
D L L F K T N R K V T V K Q L K E D Y F
                        1750 1760 1770 1780 1790
AAG AAA ATT GAA TGC TTC GAC TCT GTG GAG ATT AGC GGG GTC GAA GAT CGG TTC AAC GCA
< 1860
K K I E C F D S V E I S G V E D R F N A
                        1810 1820 1830 1840 1850
AGC CTG GGT ACC TAC CAT GAT CTG CTT AAG ATC ATC AAG GAC AAG GAT TTT CTG GAC AAT
< 1920
S L G T Y H D L L K I I K D K D F L D N
                        1870 1880 1890 1900 1910
GAG GAG AAC GAG GAC ATC CTT GAG GAC ATT GTC CTG ACT CTC ACT CTG TTC GAG GAC CGG
< 1980
E E N E D I L E D I V L T L T L F E D R
                        1930 1940 1950 1960 1970
GAA ATG ATC GAG GAG AGG CTT AAG ACC TAC GCC CAT CTG TTC GAC GAT AAA GTG ATG AAG
< 2040
E M I E E R L K T Y A H L F D D K V M K
                        1990 2000 2010 2020 2030
CAA CTT AAA CGG AGA AGA TAT ACC GGA TGG GGA CGC CTT AGC CGC AAA CTC ATC AAC GGA
< 2100
Q L K R R R Y T G W G R L S R K L I N G
                        2050 2060 2070 2080 2090
ATC CGG GAC AAA CAG AGC GGA AAG ACC ATT CTT GAT TTC CTT AAG AGC GAC GGA TTC GCT
< 2160
I R D K Q S G K T I L D F L K S D G F A
                        2110 2120 2130 2140 2150
AAT CGC AAC TTC ATG CAA CTT ATC CAT GAT GAT TCC CTG ACC TTT AAG GAG GAC ATC CAG
< 2220
N R N F M Q L I H D D S L T F K E D I Q
                        2170 2180 2190 2200 2210
AAG GCC CAA GTG TCT GGA CAA GGT GAC TCA CTG CAC GAG CAT ATC GCA AAT CTG GCT GGT
< 2280
K A Q V S G Q G D S L H E H I A N L A G
                        2230 2240 2250 2260 2270
TCA CCC GCT ATT AAG AAG GGT ATT CTC CAG ACC GTG AAA GTC GTG GAC GAG CTG GTC AAG
< 2340
S P A I K K G I L Q T V K V V D E L V K
                        2290 2300 2310 2320 2330
GTG ATG GGT CGC CAT AAA CCA GAG AAC ATT GTC ATC GAG ATG GCC AGG GAA AAC CAG ACT
< 2400
V M G R H K P E N I V I E M A R E N Q T
                        2350 2360 2370 2380 2390
ACC CAG AAG GGA CAG AAG AAC AGC AGG GAG CGG ATG AAA AGA ATT GAG GAA GGG ATT AAG
< 2460
T Q K G Q K N S R E R M K R I E E G I K
                        2410 2420 2430 2440 2450
GAG CTC GGG TCA CAG ATC CTT AAA GAG CAC CCG GTG GAA AAC ACC CAG CTT CAG AAT GAG
< 2520
E L G S Q I L K E H P V E N T Q L Q N E
                        2470 2480 2490 2500 2510
AAG CTC TAT CTG TAC TAC CTT CAA AAT GGA CGC GAT ATG TAT GTG GAC CAA GAG CTT GAT
< 2580
K L Y L Y Y L Q N G R D M Y V D Q E L D
                        2530 2540 2550 2560 2570
ATC AAC AGG CTC TCA GAC TAC GAC GTG GAC GCC ATC GTC CCT CAG AGC TTC CTC AAA GAC
< 2640
I N R L S D Y D V D A I V P Q S F L K D
                        2590 2600 2610 2620 2630
GAC TCA ATT GAC AAT AAG GTG CTG ACT CGC TCA GAC AAG AAC CGG GGA AAG TCA GAT AAC
< 2700
D S I D N K V L T R S D K N R G K S D N
                        2650 2660 2670 2680 2690
GTG CCC TCA GAG GAA GTC GTG AAA AAG ATG AAG AAC TAT TGG CGC CAG CTT CTG AAC GCA
< 2760
V P S E E V V K K M K N Y W R Q L L N A
                        2710 2720 2730 2740 2750
AAG CTG ATC ACT CAG CGG AAG TTC GAC AAT CTC ACT AAG GCT GAG AGG GGC GGA CTG AGC
< 2820
K L I T Q R K F D N L T K A E R G G L S
                        2770 2780 2790 2800 2810
GAA CTG GAC AAA GCA GGA TTC ATT AAA CGG CAA CTT GTG GAG ACT CGG CAG ATT ACT AAA
< 2880
E L D K A G F I K R Q L V E T R Q I T K
                        2830 2840 2850 2860 2870
CAT GTC GCC CAA ATC CTT GAC TCA CGC ATG AAT ACC AAG TAC GAC GAA AAC GAC AAA CTT
< 2940
H V A Q I L D S R M N T K Y D E N D K L
                        2890 2900 2910 2920 2930
ATC CGC GAG GTG AAG GTG ATT ACC CTG AAG TCC AAG CTG GTC AGC GAT TTC AGA AAG GAC
< 3000
I R E V K V I T L K S K L V S D F R K D
                        2950 2960 2970 2980 2990
TTT CAA TTC TAC AAA GTG CGG GAG ATC AAT AAC TAT CAT CAT GCT CAT GAC GCA TAT CTG
< 3060
F Q F Y K V R E I N N Y H H A H D A Y L
                        3010 3020 3030 3040 3050
AAT GCC GTG GTG GGA ACC GCC CTG ATC AAG AAG TAC CCA AAG CTG GAA AGC GAG TTC GTG
< 3120
N A V V G T A L I K K Y P K L E S E F V
                        3070 3080 3090 3100 3110
TAC GGA GAC TAC AAG GTC TAC GAC GTG CGC AAG ATG ATT GCC AAA TCT GAG CAG GAG ATC
< 3180
Y G D Y K V Y D V R K M I A K S E Q E I
                        3130 3140 3150 3160 3170
GGA AAG GCC ACC GCA AAG TAC TTC TTC TAC AGC AAC ATC ATG AAT TTC TTC AAG ACC GAA
< 3240
G K A T A K Y F F Y S N I M N F F K T E
                        3190 3200 3210 3220 3230
ATC ACC CTT GCA AAC GGT GAG ATC CGG AAG AGG CCG CTC ATC GAG ACT AAT GGG GAG ACT
< 3300
I T L A N G E I R K R P L I E T N G E T
                        3250 3260 3270 3280 3290
GGC GAA ATC GTG TGG GAC AAG GGC AGA GAT TTC GCT ACC GTG CGC AAA GTG CTT TCT ATG
< 3360
G E I V W D K G R D F A T V R K V L S M
                        3310 3320 3330 3340 3350
CCT CAA GTG AAC ATC GTG AAG AAA ACC GAG GTG CAA ACC GGA GGC TTT TCT AAG GAA TCA
< 3420
P Q V N I V K K T E V Q T G G F S K E S
                        3370 3380 3390 3400 3410
ATC CTC CCC AAG CGC AAC TCC GAC AAG CTC ATT GCA AGG AAG AAG GAT TGG GAC CCT AAG
< 3480
I L P K R N S D K L I A R K K D W D P K
                        3430 3440 3450 3460 3470
AAG TAC GGC GGA TTC GAT TCA CCA ACT GTG GCT TAT TCT GTC CTG GTC GTG GCT AAG GTG
< 3540
K Y G G F D S P T V A Y S V L V V A K V
                        3490 3500 3510 3520 3530
GAA AAA GGA AAG TCT AAG AAG CTC AAG AGC GTG AAG GAA CTG CTG GGT ATC ACC ATT ATG
< 3600
E K G K S K K L K S V K E L L G I T I M
                        3550 3560 3570 3580 3590
GAG CGC AGC TCC TTC GAG AAG AAC CCA ATT GAC TTT CTC GAA GCC AAA GGT TAC AAG GAA
< 3660
E R S S F E K N P I D F L E A K G Y K E
                        3610 3620 3630 3640 3650
GTC AAG AAG GAC CTT ATC ATC AAG CTC CCA AAG TAT AGC CTG TTC GAA CTG GAG AAT GGG
< 3720
V K K D L I I K L P K Y S L F E L E N G
                        3670 3680 3690 3700 3710
CGG AAG CGG ATG CTC GCC TCC GCT GGC GAA CTT CAG AAG GGT AAT GAG CTG GCT CTC CCC
< 3780
R K R M L A S A G E L Q K G N E L A L P
                        3730 3740 3750 3760 3770
TCC AAG TAC GTG AAT TTC CTC TAC CTT GCA AGC CAT TAC GAG AAG CTG AAG GGG AGC CCC
< 3840
S K Y V N F L Y L A S H Y E K L K G S P
                        3790 3800 3810 3820 3830
GAG GAC AAC GAG CAA AAG CAA CTG TTT GTG GAG CAG CAT AAG CAT TAT CTG GAC GAG ATC
< 3900
E D N E Q K Q L F V E Q H K H Y L D E I
                        3850 3860 3870 3880 3890
ATT GAG CAG ATT TCC GAG TTT TCT AAA CGC GTC ATT CTC GCT GAT GCC AAC CTC GAT AAA
< 3960
I E Q I S E F S K R V I L A D A N L D K
                        3910 3920 3930 3940 3950
GTC CTT AGC GCA TAC AAT AAG CAC AGA GAC AAA CCA ATT CGG GAG CAG GCT GAG AAT ATC
< 4020
V L S A Y N K H R D K P I R E Q A E N I
                        3970 3980 3990 4000 4010
ATC CAC CTG TTC ACC CTC ACC AAT CTT GGT GCC CCT GCC GCA TTC AAG TAC TTC GAC ACC
< 4080
I H L F T L T N L G A P A A F K Y F D T
                        4030 4040 4050 4060 4070
ACC ATC GAC CGG AAA CGC TAT ACC TCC ACC AAA GAA GTG CTG GAC GCC ACC CTC ATC CAC
< 4140
T I D R K R Y T S T K E V L D A T L I H
                        4090 4100 4110 4120 4130
CAG AGC ATC ACC GGA CTT TAC GAA ACT CGG ATT GAC CTC TCA CAG CTC GGA GGG GAT GAG
< 4200
Q S I T G L Y E T R I D L S Q L G G D E
                        4150 4160 4170 4180 4190
GGA GCT CCC AAG AAA AAG CGC AAG GTA GGT AGT TCC TAA < 4239
G A P K K K R K V G S S*
                        4210 4220 4230
```

TABLE 3
List of Identified Human Telomere-Associated Proteins, Related to
FIG. 1. Bolded portions are known telomere associated proteins

| | | | Average | # of |
|---|---|---|---|---|
| | Protein | | Log2- | Unique |
| Function | ID | Description | Ratio | Peptides |
| Telomere | APEX1 | apurinic/apyrimidinic endodeoxyribonuclease 1 | 2.41407 | 6 |
| Mainteinance | AURKB | aurora kinase B | 1.361768359 | 2 |
| | GAR1 | GAR1 ribonucleoprotein | 1.06101 | 3 |
| | NAT10 | N-acetyltransferase 10 | 0.82009 | 7 |
| | NBN | nibrin | 1.66482 | 4 |
| | POLD1 | DNA polymerase delta 1, catalytic subunit | 1.48504 | 3 |
| | POT1 | protection of telomeres 1 | 0.99276 | 1 |
| | RFC2 | replication factor C subunit 2 | 1.15374 | 5 |
| | RPA1 | replication protein A1 | 1.98550043 | 1 |
| | RPA2 | replication protein A2 | 2.91346 | 1 |
| | TERF2 | telomeric repeat binding factor 2 | 2.12737 | 4 |
| | TERF2IP | TERF2 interacting protein | 1.0625 | 2 |
| | UPF1 | UPF1, RNA helicase and ATPase | 0.584962501 | 10 |
| Chromatin | ATRX | ATRX, chromatin remodeler | 3.424922088 | 2 |
| Modulation | CBX5 | chromobox 5 | 1.209973162 | 5 |
| | DNAJC2 | DnaJ heat shock protein family (Hsp40) member C2 | 4.59865 | 2 |
| | H3F3A | H3 histone family member 3A | 0.59081 | 2 |
| | KDM1A | lysine demethylase 1A | 0.97819563 | 5 |
| | NOC2L | NOC2 like nucleolar associated transcriptional | 4.18059 | 5 |
| | | repressor | | |
| | SIRT1 | sirtuin 1 | 3.25938 | 3 |
| | SRPK1 | SRSF protein kinase 1 | 2.91954 | 2 |
| Cell Cycle | ASNS | asparagine synthetase (glutamine-hydrolyzing) | 0.63539 | 11 |
| | CDC73 | cell division cycle 73 | 0.91754 | 1 |
| | CDK1 | cyclin dependent kinase 1 | 0.92485 | 6 |
| | DDB1 | damage specific DNA binding protein 1 | 0.50589093 | 10 |
| | MCM5 | minichromosome maintenance complex | 1.14787 | 19 |
| | | component 5 | | |
| | MCM6 | minichromosome maintenance complex | 0.78711 | 15 |
| | | component 6 | | |
| | NOLC1 | nucleolar and coiled-body phosphoprotein 1 | 1.95093 | 3 |
| | NPM1 | nucleophosmin | 1.04433 | 5 |
| | ORC2 | solute carrier family 25 member 2 | 0.82374936 | 2 |
| | ORC5 | origin recognition complex subunit 5 | 0.847996907 | 2 |
| | PA2G4 | proliferation-associated 2G4 | 0.74259 | 13 |
| | PRIM1 | primase (DNA) subunit 1 | 3.361768359 | 1 |
| DNA | COPS4 | COP9 signalosome subunit 4 | 1.71288 | 6 |
| Damage | COPS5 | COP9 signalosome subunit 5 | 1.93392 | 2 |
| Repair | MSH2 | mutS homolog 2 | 3.26642 | 10 |
| | RAD50 | RAD50 double strand break repair protein | 1.25777 | 16 |
| | RFC3 | replication factor C subunit 3 | 1.86393845 | 2 |
| | TP53BP1 | tumor protein p53 binding protein 1 | 0.65043 | 11 |
| Transcription | ALYREF | Aly/REF export factor | 0.86393845 | 4 |
| | HDGFRP2 | HDGF like 2 | 5.44536 | 3 |
| | PPP1R10 | protein phosphatase 1 regulatory subunit 10 | 0.70197 | 1 |
| | RDBP | negative elongation factor complex member E | 4.32604 | 2 |
| | SNW1 | SNW domain containing 1 | 0.62601 | 8 |
| | TBL1XR1 | transducin beta like 1 X-linked receptor 1 | 1.90515 | 3 |
| | TCEB1 | elongin C | 3.77521 | 4 |
| | UBTF | upstream binding transcription factor, RNA | 2.58048 | 4 |
| | | polymerase I | | |
| Transport | APOE | apolipoprotein E | 1.11208 | 10 |
| | BSG | basigin (Ok blood group) | 3.16416 | 3 |
| | EXOC7 | exocyst complex component 7 | 4.31036 | 3 |
| | KHSRP | KH-type splicing regulatory protein | 0.63193 | 17 |
| | TOMM40 | translocase of outer mitochondrial membrane 40 | 0.80199 | 4 |
| | UQCRC1 | ubiquinol-cytochrome c reductase core protein I | 2.09936 | 22 |
| | UQCRC2 | ubiqumol-cytochrome c reductase core protein II | 1.10402 | 18 |
| | VDAC1 | voltage dependent anion channel 1 | 0.74959 | 11 |
| RNA Binding | AIMP2 | aminoacyl tRNA synthetase complex interacting | 0.64044 | 4 |
| | | multifunctional protein 2 | | |
| | ANP32B | acidic nuclear phosphoprotein 32 family member B | 0.61466 | 2 |
| | ANXA11 | annexin A11 | 1.24527 | 12 |
| | BYSL | bystin like | 5.24039 | 3 |
| | C4BPA | complement component 4 binding protein alpha | 1.07586 | 1 |
| | DDX21 | DEAD-box helicase 56 | 0.60164 | 14 |
| | ELAVL1 | ELAV like RNA binding protein 1 | 0.72909 | 4 |
| | HNRNPH3 | heterogeneous nuclear ribonucleoprotein H3 | 0.66352 | 3 |
| | IMP3 | signal peptide peptidase like 2A | 3.24628 | 1 |
| | NCL | nucleolin | 0.7417 | 13 |
| | NHP2L1 | small nuclear ribonucleoprotein 13 | 1.23717 | 3 |
| | NOL12 | nucleolar protein 12 | 3.75821 | 2 |
| | NOP56 | NOP56 ribonucleoprotein | 0.73285 | 12 |
| | NPM3 | nucleophosmin/nucleoplasmin 3 | 4.85971 | 2 |
| | PRPF6 | pre-mRNA processing factor 6 | 1.00303 | 12 |
| | PTBP1 | polypyrimidine tract binding protein 1 | 1.09584 | 12 |
| | PUS7 | pseudouridylate synthase 7 (putative) | 1.22331 | 3 |
| | RTCA | RNA 3′-terminal phosphate cyclase | 3.02666 | 2 |
| | SERBP1 | SERPINE1 mRNA binding protein 1 | 1.30581 | 13 |
| | SNRPE | small nuclear ribonucleoprotein polypeptide E | 0.748461233 | 2 |
| | SRSF1 | serine and arginine rich splicing factor 1 | 1.16969 | 10 |
| | THOC3 | THO complex 3 | 0.90364 | 1 |
| | THUMPD1 | THUMP domain containing 1 | 5.06122 | 3 |
| | WDR46 | WD repeat domain 46 | 4.33947 | 1 |
| Other | GLT25D1 | collagen beta(1-O)galactosyltransferase 1 | 1.05759 | 7 |
| | HSPG2 | heparan sulfate proteoglycan 2 | 1.44946 | 9 |
| | NME2P1 | NME/NM23 nucleoside diphosphate kinase 2 | 0.93276 | 5 |
| | | pseudogene 1 | | |
| | OPLAH | 5-oxoprolinase (ATP-hydrolysing) | 1.15576 | 4 |
| | RDH13 | retinol dehydrogenase 13 | 4.90304 | 2 |
| | SUN2 | Sad 1 and UNC84 domain containing 2 | 3.64546 | 3 |
| | TUBG2 | tubulin gamma 2 | 4.47379 | 1 |
| | UGGT1 | UDP-glucose glycoprotein glucosyltransferase 1 | 2.08017 | 8 |
| | ZC3HC1 | zinc finger C3HC-type containing 1 | 6.34168 | 4 |

Capture of Long-Range DNA Interactions by Biotinylated dCas9. Enhancers regulate designated promoters over distances by long-range DNA interactions, or chromatin loops. Long-range chromatin interactions have been observed by chromosome conformation capture (3C) (Dekker et al., 2002) and derivative methods including 4C (Simonis et al., 2006; Zhao et al., 2006), 5C (Dostie et al., 2006), and Hi-C (Lieberman-Aiden et al., 2009), as well as fluorescence in situ hybridization (FISH) (Osborne et al., 2004). However, these methods are either limited to pre-defined chromatin domains or of low-resolution and lacking functional details. For large-scale, de novo analysis of chromatin interactions, the ChIA-PET approach has been developed (Fullwood et al., 2009; Li et al., 2012). While this method provides unprecedented insight into the principles of 3D genomic architectures, the reliance on specific target proteins and antibodies limits its application in studying a single genomic locus.

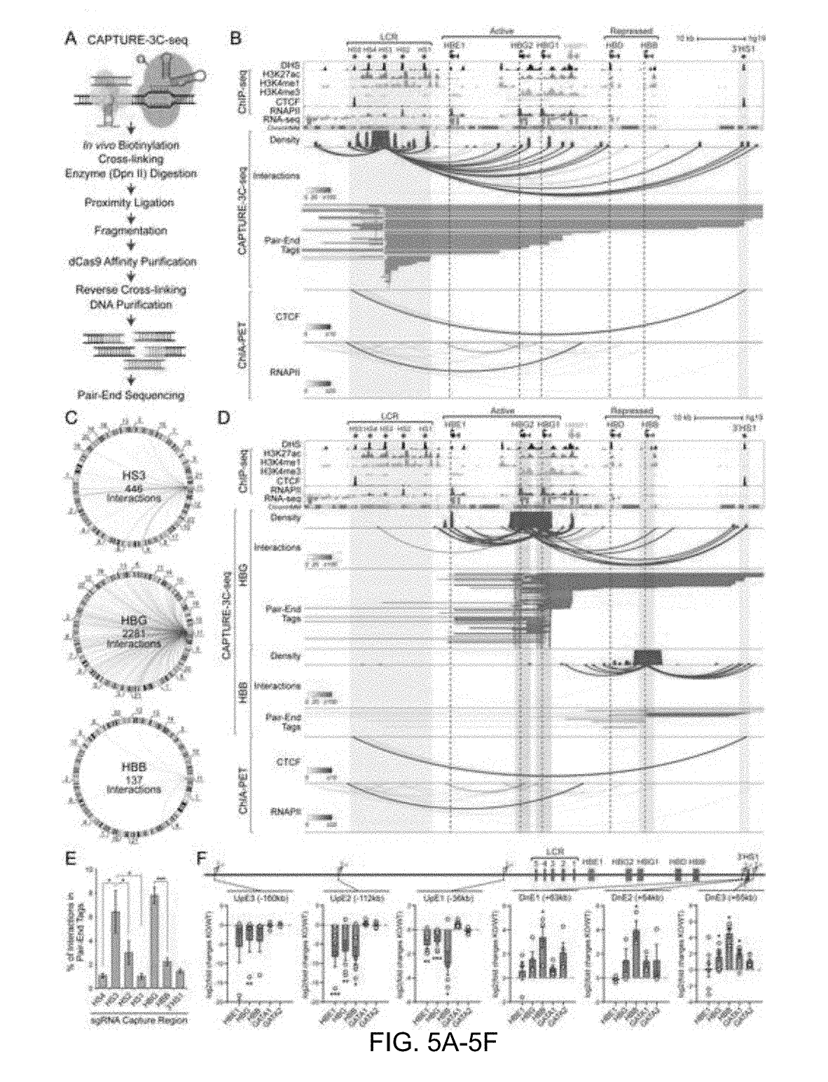

To overcome these limitations, the inventors sought to combine chromatin interaction assays with the high affinity dCas9 capture to unbiasedly identify single genomic locus-associated long-range interactions (‘CAPTURE-3C-seq’; FIG. 5A). Specifically, upon co-expression of dCas9 and sgRNAs, long-range chromatin interactions were cross-linked, followed by DpnII digestion and proximity ligation of distant DNA fragments. After fragmentation, locus-specific interactions were captured by dCas9 and analyzed by pair-end sequencing to identify the tethered long-range interactions. Of note, this approach does not involve any pre-selection steps such as PCR-based amplification (Simonis et al., 2006; Zhao et al., 2006) or oligonucleotide-based capture (Hughes et al., 2014), and all interactions brought together by dCas9-tethered DNA were captured in a single experiment.

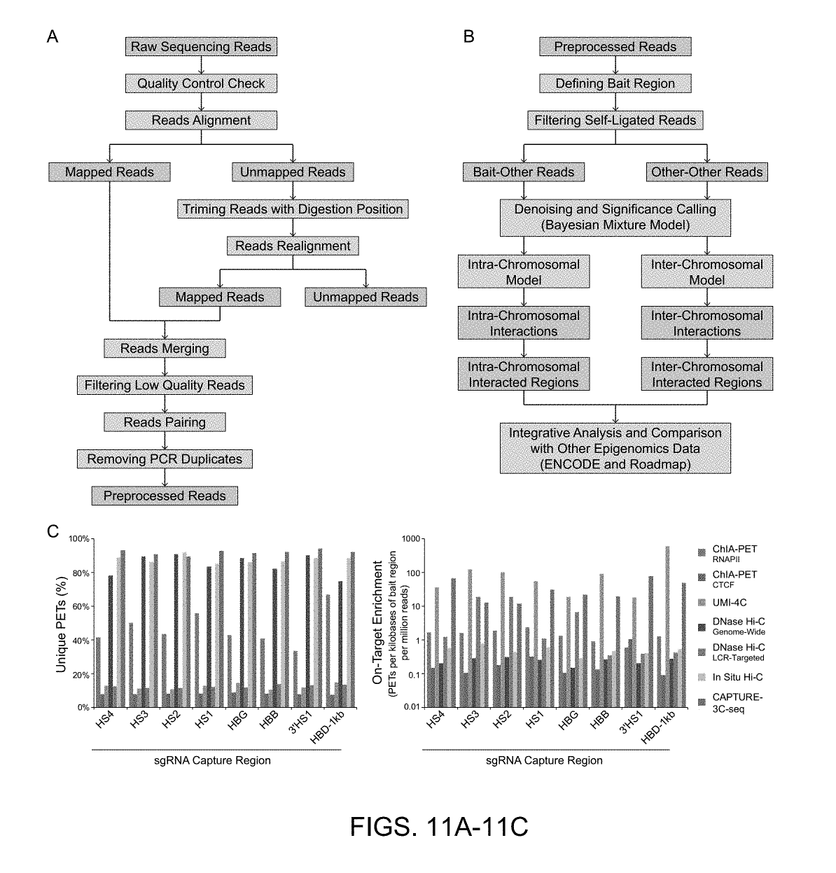

CAPTURE-chromosome conformation capture (3C)-seq (CAPTURE-3C-seq) of locus-specific DNA Interactions at β-Globin cluster. Using this approach, the inventors first identified long-range interactions at β-globin LCR by targeting dCas9 to HS3 (FIGS. 5B, 5C; Table 1). From 6,074 pair-end tags (PETs), the inventors identified 446 long-range interactions, including 232 (52.0%) intra-chromosomal interactions, 208 (46.6%) interactions within 1 Mb from HS3, and 126 (28.3%) within the β-globin cluster. To quantitatively analyze interactions, the inventors employed the FDR-controlled Bayes factor (BF) to identify ‘high-confidence interactions’ (FIGS. 11A, 11B; Method Details). Notably, the interaction frequencies were significantly higher between HS3 and the active genes (HBG1 and HBG2) than the repressed gene (HBB), suggesting that the enhancer-promoter loop formation correlates with transcriptional activities. By comparing with CTCF and RNAPII ChIA-PET data (Consortium, 2012; Li et al., 2012), the inventors identified CTCF or RNAPII-mediated interactions and many new interactions (FIG. 5B). By comparing the normalized number and frequency of interactions captured by CAPTURE-3C-seq, ChIA-PET and Hi-C, the inventors observed that CAPTURE-3C-seq displayed the highest % of unique PETs and on-target enrichment (FIG. 11C). Compared to 4C-based approach (Schwartzman et al., 2016), CAPTURE-3C-seq displayed higher % of unique PETs but comparable or slightly lower on-target enrichment (FIG. 11C).

The inventors then compared the long-range interactions at the active (HBG) and repressed (HBB) genes (FIG. 5D). CAPTURE-3C-seq of HBG revealed 215 long-range interactions connecting with most of the β-globin CREs including HS3, HBE1 and 3′HS1. Notably, 164 of 215 (76.3%) interactions were between the active HBG and HBE1 genes, whereas no interactions were detected between HBG and the repressed HBB or HBD gene, suggesting that the active genes are inter-connected and coregulated through long-range DNA interactions. By contrast, the interactions at HBB were predominantly with the proximal HBD and 3′HS1.

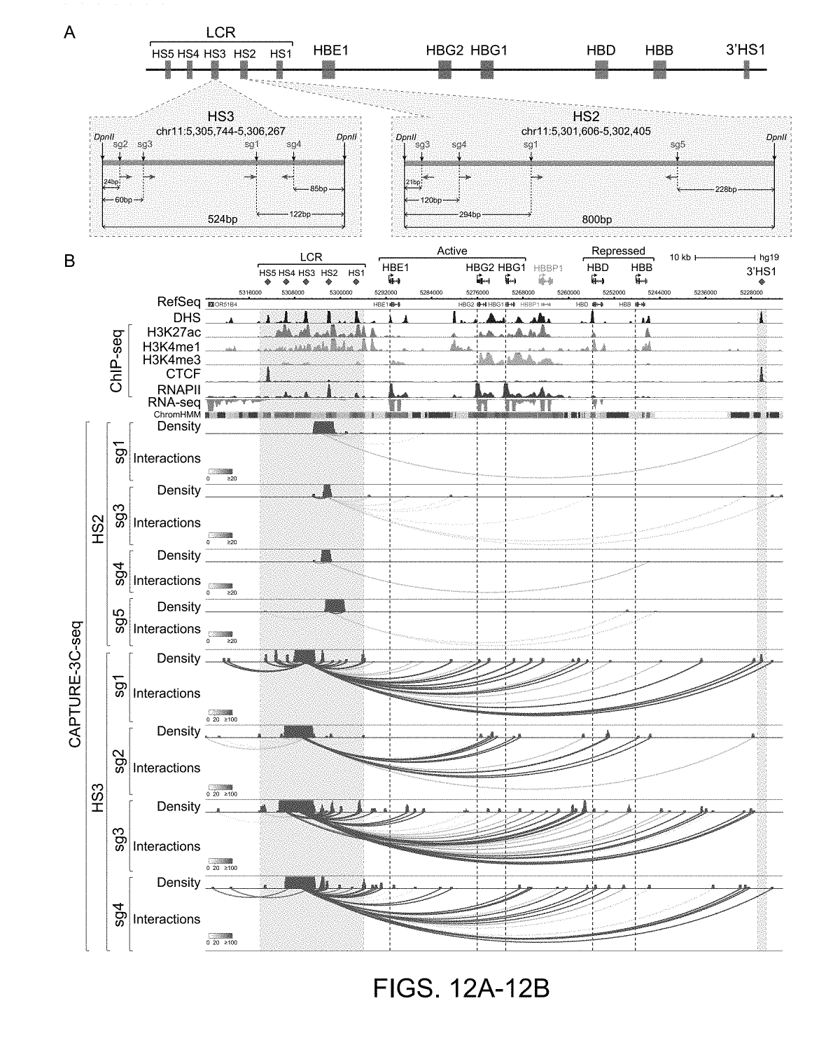

In CAPTURE-3C-seq, it is critical to rule out that the difference in the position of sgRNA target sites may cause variations in capture efficiency. Therefore, the inventors designed sgRNAs with varying distance to the DpnII site at HS2 or HS3 enhancer (FIG. 12A). Importantly, sgRNAs at various positions consistently showed higher frequency of DNA interactions at HS3 than the neighboring HS2 enhancer (FIG. 12B). Finally, the inventors compared the interactions captured at discrete β-globin CREs and identified a high-resolution, locus-specific interaction map (FIGS. 5E, 12). While some interactions were shared, most were specific to individual elements. Of note, while HS2, HS3 and HS4 are all required for β-globin gene activation (Fraser et al., 1993; Morley et al., 1992; Navas et al., 1998), HS2 and HS4 contained many fewer interactions than HS3 (FIGS. 5E, 12, 13), showing that they may cooperate through distinct regulatory composition.

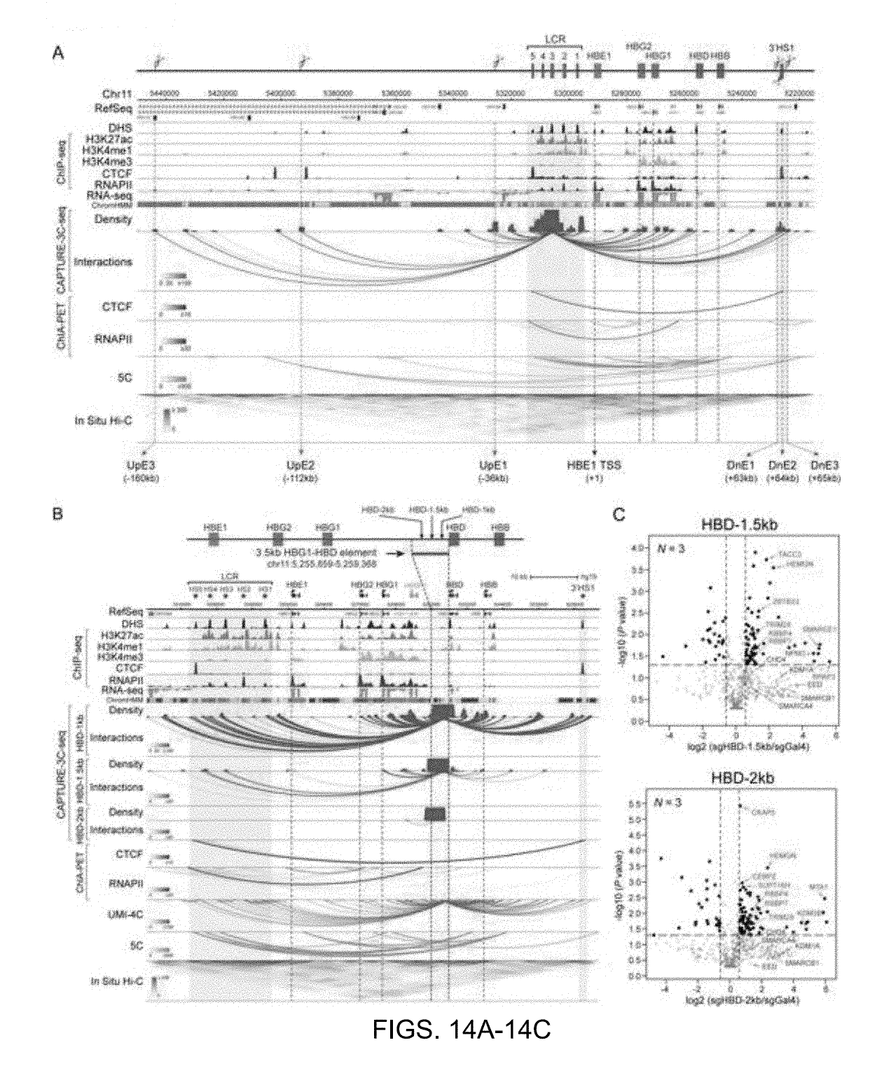

Identification of De Novo CREs for β-Globin Genes. Through unbiased capture of HS3, the inventors identified several de novo CREs with unknown roles in globin gene regulation (FIGS. 5F, 14A). By CRISPR-mediated knockout (KO) using paired sgRNAs, the inventors observed that deletion of the UpE3 element located 160 kb upstream of HBE1 led to significant downregulation of β-globin mRNAs (FIG. 5F). Similarly, KO of UpE2 (−112 kb) and UpE1 (−36 kb) resulted in significant downregulation of β-globin genes. By contrast, KO of three downstream elements (DnE1, DnE2 and DnE3) overlapping with the CTCF-associated insulator resulted in significant upregulation of the repressed HBB gene, whereas the expression of HBE1, HBG, GATA1 and GATA2 remained largely unaffected. The identification of new β-globin CREs illustrates the presence of additional distal cis-elements not recapitulated in studies using mouse models (Hardison et al., 1997; Navas et al., 1998; Peterson et al., 1998).

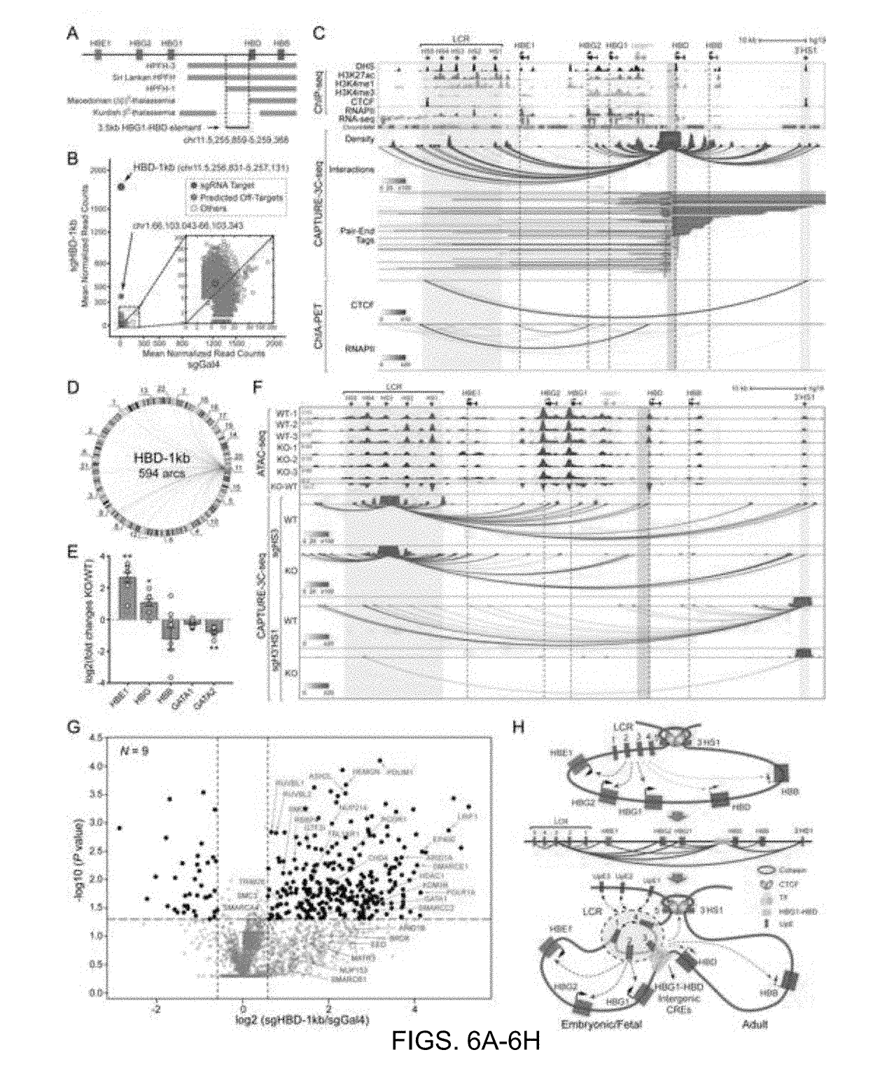

In Situ CAPTURE of A Disease-Associated CRE. Disease-associated CREs are commonly recognized by correlative chromatin features, yet limited insight has been gained into their regulatory composition. One example is the 3.5 kb HBG1-HBD intergenic region required for the silencing of fetal β-globin genes (FIG. 6A). Genetic mapping studies showed that deletion of this region in humans, including in hereditary persistence of fetal hemoglobin 1 (HPFH-1), HFPH-3 and Sri Lankan HPFH patients, led to reactivation of HBG. By contrast, in patients that retained the intergenic region, including Macedonian (δβ)⁰-thalassemia and Kurdish β⁰-thalassemia, HBG silencing was maintained (Sankaran et al., 2011). While these studies established the HBG1-HBD intergenic region as a critical disease-associated CRE, the underlying regulatory components remained unclear.

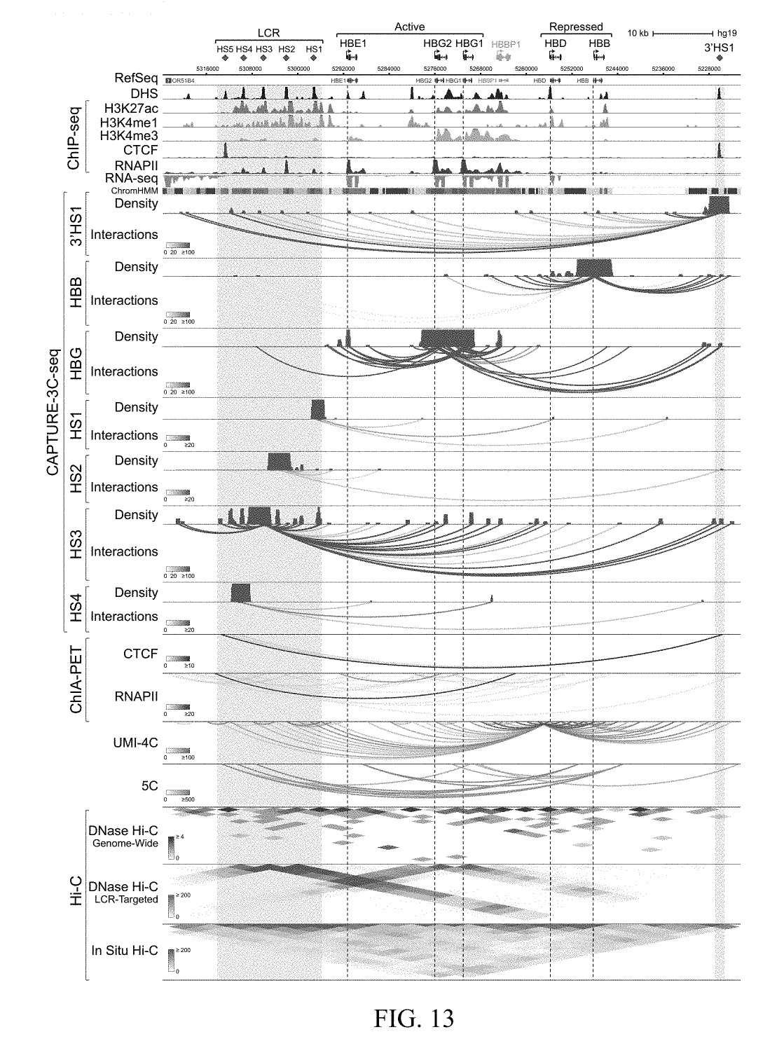

FIG. 13 shows the CAPTURE-3C-seq of Locus-Specific DNA Interactions at Multiple β-Globin CREs, Related to FIG. 5. Browser view of the long-range DNA interaction profiles at dCas9-captured β-globin CREs is shown (chr11:5,222,500-5,323,700; hg19). Contact profiles compiled from two or three CAPTURE-3C-seq experiments including the density map and interactions (or loops) are shown. ChIA-PET (Consortium, 2012; Li et al., 2012), UMI-4C (Schwartzman et al., 2016), 5C (Naumova et al., 2013), DNase Hi-C (Ma et al., 2015), in situ Hi-C(Rao et al., 2014), DHS, ChIP-seq, RNA-seq, and ChromHMM data are shown for comparison.

Therefore, the inventors designed three sgRNAs targeting the 3.5 kb HBG1-HBD intergenic element (HBD-1kb, HBD-1.5kb and HBD-2kb; FIG. 14B). The specificity of the sgRNAs was confirmed by CAPTURE-ChIP-seq (FIG. 6B). By CAPTURE-3C-seq, the inventors observed that the HBD-1kb region contained significantly higher frequency of long-range interactions than the neighboring HBD-1.5kb and HBD-2kb regions (FIG. 14B). These interactions connected HBD-1kb with most β-globin CREs, including the HS1 to HS4 enhancers, β-globin genes and insulators (FIGS. 6C,6D). Notably, KO of HBD-1kb in K562 cells resulted in upregulation of HBE1 and HBG, whereas HBB was largely unaffected (FIG. 6E). HBD-1kb KO also led to marked decreases in chromatin accessibility at the HBG and HBD promoters, HS1, HS2, and HS4 enhancers, and 3′HS1 (FIG. 6F). Furthermore, by CAPTURE-3C-seq, the inventors observed significant changes in the frequency of long-range interactions at several CREs (FIG. 6F), suggesting that the HBG1-HBD intergenic region is required for the proper chromatin configuration and the expression of β-globin genes.

By CAPTURE-Proteomics of the HBG1-HBD intergenic region, the inventors identified components of the SWI/SNF and NuRD complexes, transcriptional co-activators (EP400, KDM3B and ASH2L), co-repressors (RCOR1, TBL1XR1, LRIF1 and TRIM28/KAP1), cohesin (SMC3), nucleoporins (NUP153 and NUP214) and TFs (GATA1 and STAT1) (FIG. 6G). The identification of the SWI/SNF and cohesin proteins is consistent with their function in regulating chromatin looping (Kagey et al., 2010; Kim et al., 2009b). The presence of co-activators and co-repressors may be related to the interactions with both active and repressed β-globin genes (FIG. 6C). Notably, most of the HBD-1kb-associated proteins were not identified at the neighboring HBD-1.5kb or HBD-2kb region (FIG. 14C).

Together, these studies show a refined model for the spatial organization of the (3-globin CREs (FIG. 6H). The β-globin genes are coordinately regulated in an insulated neighborhood between HS5 and 3′HS1. The HBG1-HBD intergenic region functions as a major interaction hub linking enhancers and insulators to establish two subdomains: an embryonic/fetal subdomain containing HBE1, HBG1 and HBG2 genes, and an adult subdomain containing HBD and HBB. HS2 and other LCR enhancers cooperate with associated regulators to activate the embryonic/fetal or adult genes in a developmental stage-specific manner. Thus, the in-depth analyses of locus-specific interactions at the β-globin cluster by in situ CAPTURE not only reveal new spatial features for the composition-based hierarchical control of a lineage-specific enhancer cluster, but establish new approaches for molecular dissection of disease-associated CREs.

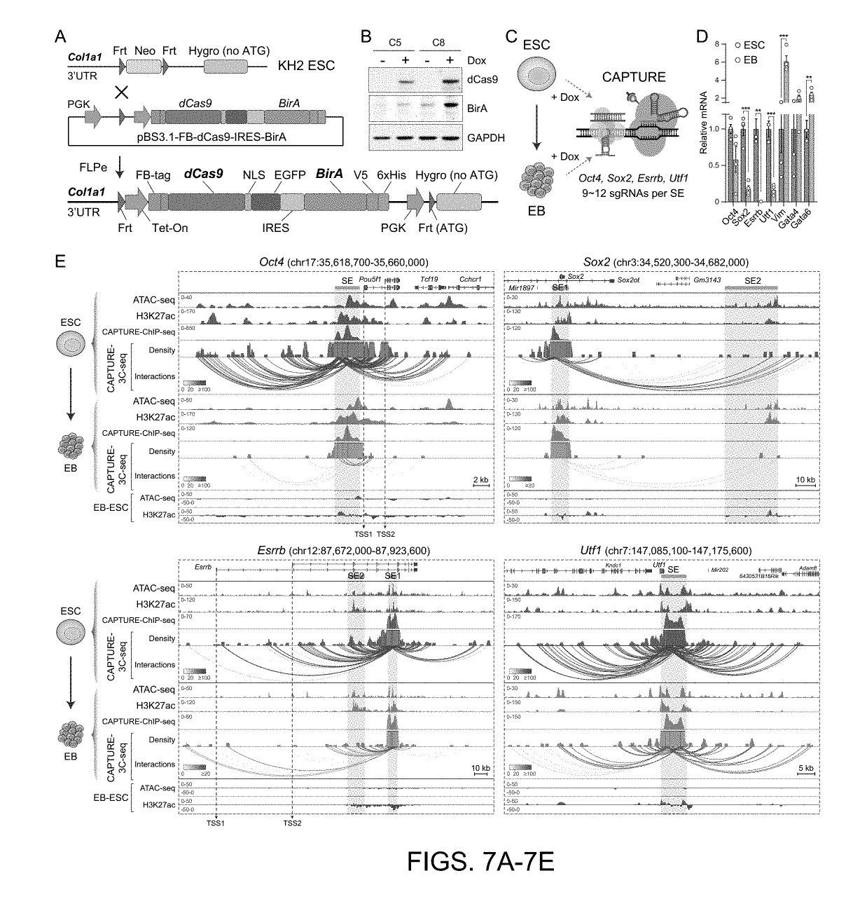

In Situ CAPTURE of Developmentally Regulated SEs. To demonstrate the utility of CAPTURE across cell models, the inventors analyzed lineage-specific SEs during mouse ESC differentiation. The inventors generated a site-specific knock-in allele containing FB-dCas9-EGFP and BirA through FLPe-mediated recombination (Beard et al., 2006) (FIG. 7A). After confirming the doxycycline (Dox)-inducible expression of dCas9 and BirA proteins (FIG. 7B), ESCs were differentiated to embryoid bodies (EBs). The inventors designed multiplexed sgRNAs targeting four ESC-specific SEs (Oct4, Sox2, Esrrb and Utf1; FIG. 7C). Upon differentiation, the expression of the SE-linked genes was significantly downregulated (FIG. 7D). The inventors then analyzed SE-associated long-range interactions and chromatin features (FIG. 7E). Strikingly, in situ CAPTURE of distinct SEs revealed frequent long-range interactions between SEs and their gene targets in ESCs, whereas the interactions were significantly less or absent in EBs. More importantly, the significant changes in SE-mediated long-range interactions, together with minimal or no changes in chromatin accessibility or H3K27ac, demonstrate that the loss of enhancer-promoter contacts precedes changes in chromatin landscape during differentiation. These findings show a model in which enhancer-promoter loop formation causally underlies gene activation (Deng et al., 2012; Deng et al., 2014). Many long-range interactions were between different SEs (Sox2 and Esrrb; FIG. 7E) or between SEs and promoters of transcript variants (Oct4 and Esrrb). Furthermore, while most long-range interactions were absent or weakened in EBs, some were maintained, indicating a dynamic and hierarchal regulation of SE interactions in response to cellular differentiation. Taken together, these studies demonstrate that the CAPTURE approaches work effectively in human cells and transgenic mouse ESCs, raising the prospect of using biotinylated dCas9 in purification of CRE-associated chromatin interactions across cellular conditions in situ and in developing tissues in vivo.

In Situ CAPTURE of Locus-Specific Interactions. Current technologies in studying chromatin structure rely on 3D genome mapping approaches. The basic principle is nuclear proximity ligation that allows detection of distant interacting DNA tethered together by higher order architectures. ChIA-PET was designed to detect genome-wide chromatin interactions mediated by specific protein factors. Hi-C was developed to capture all chromatin contacts particularly large-scale structures including the topologically associated domains (TADs) (Dixon et al., 2012); however, it lacked the level of resolution required for locus-specific interactions as well as the information of the trans-acting factors mediating such interactions. Hence, the CAPTURE method provides a complementary approach for high-resolution, unbiased analysis of locus-specific proteome and 3D interactome that is not dependent on predefined proteins, available reagents, or a priori knowledge of the target loci. The CAPTURE approach has several unique features, including the ability to specifically detect macromolecules at an endogenous locus with minimal off-targets, to identify combinatorial protein-DNA interactions, and to dissect the disease-associated or developmentally regulated cis-elements.

Important Considerations for In Situ CAPTURE. For selective capture of locus-specific chromatin interactions, the following parameters need to be carefully evaluated. First, the sgRNA target sequences should locate in close proximity to the captured element to maximize the capture efficiency, but not overlap with TF binding sites to avoid interference with protein-DNA interactions. Second, the on-target enrichment and genome-wide specificity by independent sgRNAs should be evaluated to minimize off-targets. Third, the study of locus-specific proteome requires the identification of non-specific proteins in control cells for quantitative and statistical analysis. Fourth, the analysis of CRE-mediated long-range DNA interactions requires the design of sgRNAs in close proximity to DpnII sites. Finally, the use of multiplexed sgRNAs targeting multiple CREs at the same enhancer or multiple enhancers helps distinguish consistent interactions from rare interactions of individual sgRNAs; however, the selection of multiplexed sgRNAs requires comparable on-target enrichment for each sgRNA to minimize variation in capture efficiency.

Multiplexed CAPTURE of SE Composition. Intensively marked clusters of enhancers or SEs have been described, yet the underlying principles of enhancer clustering remained unclear. Here the inventors focus on an erythroid-specific SE, or LCR, controlling the expression of β-globin genes. The β-globin LCR consists of five DHS, three of which display enhancer activities. Specifically, HS2 behaves as a classical enhancer in reporter assays (Fraser et al., 1993; Morley et al., 1992), whereas the enhancer activities of HS3 and HS4 can only be detected in the context of chromatin (Hardison et al., 1997; Navas et al., 1998). By in situ capture of β-globin CREs, these studies uncover distinguishing features in the regulatory composition of SE constituents. Importantly, the HBG and HBB promoters shared many interacting proteins and clustered closely, whereas the HS1, HS3 and HS4 enhancers clustered to form a distinct subdomain. HS2 shared interacting proteins with both subdomains. Furthermore, HS3 contains significantly more long-range interactions than the nearby enhancers. Hence, these results show a model for the hierarchical organization of the β-globin LCR, in which HS2 functions as a conventional enhancer by providing binding sites for trans-acting factors, whereas HS3 mediates long-range chromatin looping. Hence, the SE constituents cooperate through distinct regulatory composition to function within the same SE cluster. These findings also help explain the distinct requirement of HS2 and HS3 for the transgenic versus endogenous β-globin gene expression. Thus, the CAPTURE approach provides a platform for the systematic dissection of SE constituents and the underlying formative composition controlling enhancer structure-function.

Finally, the CAPTURE system can be adapted for multiplexed analysis of multiple CREs at the same enhancer or multiple enhancers, thus allowing for high-throughput capture of locus-specific interactions. High-resolution, multiplexed analysis of chromatin interactions at developmentally regulated enhancers provides evidence for the causality of chromatin looping and enhancer activities. Conversely, unbiased analysis of promoter-associated interactions will help identify the complete set of constitutive or tissue-specific distal CREs, thus allowing for comprehensive analysis of regulatory CREs of any gene. The vast majority of disease-associated variants reside within non-coding elements and exert effects through long-range regulation of gene expression. The unbiased analysis of chromatin-templated hierarchical events will help define the underlying regulatory principles, thus advancing the mechanistic understanding of the non-coding genome in human disease.

Cells and Cell Culture. Human female K562 cells were obtained from ATCC and cultured in IMDM medium containing 10% FBS and 1% penicillin/streptomycin. pEF1α-FB-dCas9 and pEF1α-BirA-V5 vectors were co-transfected into K562 cells by nucleofection using the ECM 830 Square Wave Electroporation System (Harvard Apparatus, Holliston, Mass.). Cells were plated in 96-well plates and treated with 1 μg/ml of puromycin (Sigma) and 600 μg/ml of G418 (Sigma) 48-72 hour post-transfection. Single-cell-derived clones were isolated and examined by Western blot analysis to screen for FB-dCas9 and BirA-expressing stable clones. Human primary adult erythroid progenitor cells were generated ex vivo from CD34+ HSPCs as previously described (Huang et al., 2016). Primary HSPCs from both sexes were used in this study. For inhibition of BRD4, K562 or primary human erythroid progenitor cells were treated with the vehicle control (DMSO), JQ1 (0.25 μM or 1 μM) for 2 or 6 hours before harvesting for ChIP-seq or qRT-PCR analyses. Mouse male embryonic stem cells (ESCs) were cultured on primary embryonic fibroblasts and differentiated to embryoid bodies (EBs) by LIF withdrawal for 8 days. All cultures were incubated at 37° C. in 5% CO2. All cell lines were tested for mycoplasma contamination. No cell lines used in this study were found in the database of commonly misidentified cell lines that is maintained by ICLAC and NCBI BioSample.

sgRNA Cloning and Transduction. Single guide RNAs (sgRNAs) for site-specific targeting of genomic regions were designed to minimize off-target cleavage based on publicly available filtering tools (crispr.genome-engineering.org/crispr/). To minimize potential interference between dCas9 and trans-acting factors, sgRNAs were designed to target the proximity of cis-elements. The inventors also adapted an optimized sgRNA design by including the A-U pair flip and a 5 bp extension of the hairpin as previously described (Chen et al., 2013). The sgRNAs were cloned into the lentiviral U6-driven expression vector by amplifying the insertions using a common reverse primer and unique forward primers containing the protospacer sequence, as previously described (Chen et al., 2013). Briefly, the forward primers were mixed with equal amount of reverse primer to PCR amplify sgRNA fragments using pSLQ1651 vector as the template. The PCR amplicon and the sgRNA vector containing a mCherry reporter gene were digested by restriction enzymes BstXI and XhoI for 3 hours. The digestion DNA were then purified, and ligated to the digested sgRNA vector using T4 DNA ligase. Insertion of sgRNA was validated by Sanger sequencing. Lentiviruses containing sgRNAs were packaged in HEK293T cells as previously described (Huang et al., 2016). Briefly, 2 μg of pΔ8.9, 1 μg of VSV-G and 3 μg sgRNA vectors were co-transfected into HEK293T cells seeded in 10 cm petri dish. Lentiviruses were harvested from the supernatant 48-72 hours post-transfection. FB-dCas9 and BirA-expressing K562 stable cells were then transduced with sgRNA-expressing lentiviruses in 6-well plates. To maximize sgRNA expression, the top 1% of mCherry-positive cells were FACS sorted 48 hours post-transfection. The sequences for all sgRNAs used in this study are listed in Table 2.

CAPTURE-ChIP-seq. Streptavidin Affinity Purification of dCas9-Captured DNA and Sequencing. 1×10⁷ FB-dCas9/BirA-expressing K562 stable cells transduced with sequence-specific or non-targeting sgRNAs were harvested, cross-linked with 1% formaldehyde for 10 min, and quenched with 0.125 M of glycine for 5 minutes. Cells were lysed in 1 ml RIPA buffer (10 mM Tris-HCl, 1 mM EDTA, 0.1% sodium deoxycholate, 0.1% SDS, 1% Triton X-100, pH 8.0), and rotated for 15 minutes at 4° C. Cell lysates were centrifuged at 2,300×g for 5 minutes at 4° C. to isolate the nuclei. Nuclei were suspended in 500 μl of 0.5% SDS lysis buffer (0.5% SDS, 10 mM EDTA, 50 mM Tris-HCl, pH 8.0) and subjected for sonication to shear chromatin fragments to an average size between 200 bp and 500 bp on the Branson Sonifier 450 ultrasonic processor (20% amplitude, 0.5 second on 1 second off for 30 seconds). Fragmented chromatin was centrifuged at 16,100×g for 10 minutes at 4° C. 450 μl of supernatant was transferred to a new Eppendorf tube and added final concentration 300 mM NaCl. Supernatant was then incubated with 10 μl of MyOne Streptavidin Ti Dynabeads (Thermo-Fisher Scientific) at 4° C. overnight. After overnight incubation, Dynabeads were washed twice with 1 ml of 2% SDS, twice with 1 ml of RIPA buffer with 0.5 M NaCl, twice with 1 ml of LiCl buffer (250 mM LiCl, 0.5% NP-40, 0.5% sodium deoxycholate, 1 mM EDTA and 10 mM Tris-HCl, pH 8.0), and twice with 1 ml of TE buffer (10 mM Tris-HCl, 1 mM EDTA, pH 8.0). The chromatin was eluted in SDS elution buffer (1% SDS, 10 mM EDTA, 50 mM Tris-HCl, pH 8.0) followed by reverse cross-linking at 65° C. overnight. The ChIP DNA was treated with RNase A (5 μg/ml) and protease K (0.2 mg/ml) at 37° C. for 30 minutes, and purified using QIAquick Spin columns (Qiagen). 1 ng of ChIP DNA was processed for library generation using the NEBNext ChIP-seq Library Prep Master Mix (New England Biolabs or NEB) following the manufacturer's protocol. Libraries were pooled and sequenced on an Illumina Nextseq500 system using the 75 bp high output sequencing kit.

CAPTURE-ChIP-seq Data Analysis.

ChIP-seq raw reads were aligned to human (hg19) or mouse (mm9) genome assembly using Bowtie1 (Langmead et al., 2009) with default parameters. The first 10 nucleotides and the last 3 nucleotides from each read were excluded from alignment. For all ChIP-seq samples except sgHBG, only reads that can be uniquely mapped to the genome were used for further analysis. For sgHBG samples, since the sequences of HBG1 and HBG2 genes are highly similar, the inventors kept reads with two alignments. MACS was applied to each sample to perform peak calling using the “--nomodel” parameter (Zhang et al., 2008). Peaks that overlap with the blacklist regions annotated by the ENCODE project (Consortium, 2012), the repeat masked region (chr2:33,141,250-33,142,690; hg19), or the validated non-targeting control sgRNA (sgGal4) enriched regions (chr6:119,558,373-119,558,873, chr17:42,074,844-42,075,323, chr21:15,457,141-15,457,641, chr20:26,188,800-26,190,400, chr17:42,074,844-42,075,323 and chr1111:192,110-192,410; hg19) were removed. To compare ChIP-seq signal intensities in samples prepared from cells expressing the target-specific sgRNAs versus the non-targeting sgGal4, MAnorm (Shao et al., 2012) was applied to remove systematic bias between samples and then calculate the normalized ChIP-seq read densities of each peak for all samples. The window size was 300 bp which matched the average width of the identified ChIP-seq peaks.

CAPTURE-ChIP-qPCR.

For CAPTURE-ChIP-qPCR analysis, 0.5 to 1×10⁷ FB-dCas9/BirA K562 stable cells transduced with sgTelomere were used. The captured DNA was isolated using the protocol described for CAPTURE-ChIP-seq except was analyzed by quantitative PCR (qPCR). For input samples, 80 μl of SDS elution buffer was added into 20 μl of the sheared chromatin. The samples were incubated at 65° C. overnight to reverse cross-linking. DNA fragments were purified with the QIAquick PCR Purification Kit and eluted with 100 μl of EB buffer. Primers targeting human telomere sequences or a single copy gene 34B11 as a control were used for qPCR analysis. Primer sequences are listed in Table 2.

CAPTURE-Proteomics. The inventors performed multiplexed isobaric tag for relative and absolute quantitation (iTRAQ)-based quantitative proteomic analysis of the isolated protein complexes. Briefly, the trypsin-digested peptides were labeled with 4-plex iTRAQ reagents (AB Sciex). After labelling, all peptides were mixed and loaded into an online three dimensional chromatography platform for in-depth proteome quantification as previously described (Zhou et al., 2013) with the following modifications. First, the inventors performed in-solution, on-bead digestion of the purified samples to minimize sample loss associated with gel-based protocols. Second, the inventors used the high-pH reversed phase (RP) and strong anion exchange separation stages coupled with a narrow-bore low-pH RP analytical column to achieve extreme separation of peptides in a nanoflow regime. Third, the inventors chose the final dimension column geometry to maintain the integrity of chromatographic separation at ultra-low effluent flow rates to maximize electrospray ionization efficiency. Finally, the inventors implemented all separation stages in microcapillary format coupled to the spectrometer, thus providing automated, efficient capture and transfer of peptides.

dCas9 Affinity Purification.

0.25 to 1×10⁹ FB-dCas9/BirA K562 stable cells transduced with sequence-specific sgRNAs or non-targeting sgRNA (sgGal4) were harvested, cross-linked with 2% formaldehyde for 10 minutes, and quenched with 0.25 M of glycine for 5 minutes. Cells were washed twice with PBS, lysed with 10 ml of cell lysis buffer (25 mM Tris-HCl, 85 mM KCl, 0.1% Triton X-100, pH 7.4, freshly added 1 mM DTT and 1:200 proteinase inhibitor cocktail (Sigma)), and rotated for 15 minutes at 4° C. Cell lysates were centrifuged at 2,300×g for 5 minutes at 4° C. to isolate the nuclei. The nuclei were resuspended in 5 ml nuclear lysis buffer (50 mM Tris-HCl, 10 mM EDTA, 4% SDS, pH 7.4, freshly added 1 mM DTT and 1:200 proteinase inhibitor cocktail) and incubated for 10 minutes at room temperature. Nuclei suspension was then mixed with 15 ml of 8 M urea buffer and centrifuged at 16,100×g for 25 minutes at room temperature. Nuclei pellets were then resuspended in 5 ml nuclear lysis buffer and mixed with 15 ml of 8 M urea buffer, and centrifuged at 16,100×g for 25 minutes at room temperature. The samples were washed twice more in 5 ml nuclear lysis buffer and mixed with 15 ml of 8 M urea buffer, followed by centrifugation at 16,100×g for 25 minutes at room temperature. Pelleted chromatin was then washed twice with 5 ml cell lysis buffer. Chromatin pellet was resuspended in 5 ml of IP binding buffer without NaCl (20 mM Tris-HCl, 1 mM EDTA, 0.1% NP-40, 10% glycerol, pH 7.5, freshly added proteinase inhibitor) and aliquoted into Eppendorf tubes. Chromatin suspension was then subjected to sonication to an average size ˜500 bp on the Branson Sonifier 450 ultrasonic processor (10% amplitude, 0.5 second on 1 second off for 1 minute). Fragmented chromatin was centrifuged at 16,100×g for 25 minutes at 4° C. Supernatant was combined and final concentration 150 mM NaCl was added to the sheared chromatin. To prepare the streptavidin beads for affinity purification, 250 μl to 1 ml of streptavidin agarose slurry (Life Technologies) was washed 3 times in 1 ml of IP binding buffer and added to soluble chromatin. After overnight incubation at 4° C., streptavidin beads were collected by centrifugation at 800×g for 3 minutes at 4° C. The beads were then washed 5 times with 1 ml of IP binding buffer (20 mM Tris-HCl, 1 mM EDTA, 0.1% NP-40, 10% glycerol, 150-300 mM NaCl, pH 7.5, freshly added proteinase inhibitor) and resuspended in 100 μl of 1× XT sample loading buffer (Bio-Rad) containing 1.25% 2-mercaptoethanol followed by incubation at 100° C. for 20 minutes. The proteins were separated by SDS-PAGE and analyzed by Western blot.

In-Solution Digestion and Peptide Isolation. To improve the sensitivity and minimize sample loss associated with in-gel digestion, the inventors performed in-solution on-beads trypsin digestion. Briefly, after overnight incubation of streptavidin beads with chromatin, the beads were washed 5 times with detergent-free IP binding buffer (20 mM Tris-HCl, 1 mM EDTA, 150 mM NaCl, 10% glycerol, pH 7.5). The beads were resuspended in 500 μl of 0.5 M Tris (pH 8.5) and incubated with final concentration 20 mM TCEP (tris(2-carboxyethyl)phosphine, Sigma, made freshly as 0.5M stock in 2M NaOH) at room temperature for 1 hour. The beads were then mixed with 4 μl of MMTS (S-Methyl methanethiosulfonate, Sigma) and incubated for 20 minutes at room temperature. The beads suspension was then digested with 20 μg of Trypsin (Promega) at 37° C. overnight. After trypsin digestion, the beads were loaded to the cellulose acetate filter spin cup (0.45 μm pore size, Pierce) and centrifuged at 12,000×g for 2 minutes at room temperature to collect flow-through containing peptides. The peptide solution was mixed with final concentration 3 M NaCl and boiled at 95° C. for 1 hour to reverse formaldehyde cross-linking. Digested peptides were dried using a SpeedVac (Thermo-Fisher Scientific), reconstituted in 200 μl of 0.1% trifluoroacetic acid (TFA) and loaded onto a pre-equilibrated Oasis HLB elute plate (Waters Corporation). After discarding the flow-through, the columns were washed with 800 μl of 0.1% TFA, followed by another wash with 200 μl of ddH₂O. The desalted peptides were then eluted with 50 μl of 70% acetonitrile and labeled with multiplexed isobaric tags using the iTRAQ Reagents-4Plex Multiplex Kit (SCIEX) according to the manufacturer's protocol.

Multi-Dimension Separation and Data Acquisition.

Nanoscale three dimensional online chromatography platform consists of first dimension reversed phase (RP) column (100 μm I.D. capillary packed with 10 cm of 5 μm dia. XBridge (Waters Corp., Milford, Mass.) C18 resin), second dimension strong anion exchange (SAX) column (100 μm I.D. 10 cm of 10 μm dia. POROS10HQ (AB Sciex, Foster City, Calif.) resin) and third dimension reversed phase column (15 μm I.D. 50 cm of 3 μm dia. Monitor C18 (Column Engineering, Ontario, Calif.), integrated 1 μm dia. emitter tip). The final dimension ran at 1-2 nL/min with a ˜280 min gradient from 2% B to 50% B (A=0.1% formic acid, B=acetonitrile with 0.1% formic acid). The downstream TripleTOF 5600+(AB Sciex, Foster City, Calif.) was set in data-dependent acquisition (DDA) mode for data acquisition. Top 50 precursors (charge state +2 to +4, >70 counts) in each MS scan (800 ms, scan range 550-1500 m/z) were subjected to MS/MS (maximum time 250 ms, scan range 100-1400 m/z). Electrospray voltage was 2.4 kV.

Data Processing and Protein Quantification.

The mass spectrometry data was subjected to search against SwissProt database (downloaded on Oct. 2, 2016) with ProteinPilot V4.5 (AB Sciex, Framingham, Mass.). Official HGNC Gene Symbols were included in the database. The search parameter was set to “iTRAQ 4-plex (peptides labeling) with 5600 TripleTOF”. In this study, the inventors also removed peptides that can be assigned to more than one gene. The peptide spectra match (PSM) false discovery rate (FDR) was used to filter the peptides identified for further analysis. Specifically, FDR is the statistical model used to evaluate the confidence level of peptide identification based on the well-established target-decoy search strategy (Elias and Gygi, 2007). The target-decoy search strategy requires repeated search using identical parameters against a ‘decoy’ database in which the target sequences have been reversed or randomized. The number of matches found in ‘decoy’ database is used as an estimate of the number of false positives (FP) that are present in the ‘target’ database. The number of true positive (TP) matches in the ‘target’ database and the number of FP matches in the ‘decoy’ database are then used to calculate the False Discovery Rate (FDR)=FP/(FP+TP). Only those peptides with scores at or below a PSM FDR threshold of 1% were kept for data analysis. After that, the inventors summed the intensity of each iTRAQ reporter ion for the peptides that can only be assigned to single gene to generate the iTRAQ intensity value for each gene. The inventors then removed genes with weak quantification signal (total signal intensity of iTRAQ reporter ions ≤50). To compare between independent experiments and individual samples, the ion intensity of iTRAQ mass spectrometry signal was normalized based on the cumulative intensity of the high-confidence non-specific proteins (FIG. 9B) identified from four control cell lines expressing the non-targeting sgRNAs (sgGal4) and/or dCas9 and the bait protein (dCas9). Specifically, for each individual target-specific sgRNA and the corresponding control samples, the log 2 ratios of iTRAQ reporter ion intensities of all detected non-specific proteins were plotted against the average intensities between two profiles. The principal component analysis (PCA) was applied to the plot to not only rescale the average log 2 ratios of these proteins to zero, but also minimize the total variation of observed log 2 ratios. Then the principal components were applied to the log 2 ratios and the average intensities of all detected proteins, and the projection of their log 2 ratios to the second principal component was taken as the normalized log 2 ratios of iTRAQ intensities between two profiles. After the global normalization of each sample, the ratios of the iTRAQ reporter ion intensity for each protein in target-specific sgRNA samples relative to the non-targeting sgGal4 sample were collected across replicate experiments. Only proteins detected in at least 3 replicates (at least 2 replicates for sgHBD-1.5kb and sgHBD-2kb) were subjected to statistical analysis, in which a P value was calculated to measure the statistical significance of the log 2 iTRAQ ratios of each identified protein in the replicate experiments by paired t-test. After removing the non-specific proteins identified from control experiments, the iTRAQ ratio and P value for the remaining proteins were calculated in each replicate experiment. To determine the ratio and P value cutoffs used to identify significantly enriched locus-specific proteins, the inventors surveyed the distribution of the “high-confidence non-specific proteins” in all proteomic experiments, and observed that 78.3% and 79.8% of the ‘high-confidence non-specific proteins’ displayed iTRAQ ratio less than 1.5-fold and P value more than 0.05 (FIG. 9C). Based on these analyses, a protein was considered to be significantly enriched if the iTRAQ ratio ≥1.5 and P value ≤0.05 in samples prepared from cells expressing sequence-specific sgRNAs versus the non-targeting sgGal4 control.

Connectivity Network Analysis.

The connectivity network was built by Gephi (version 0.9.1) using all interactions between the dCas9-captured locus-specific proteins and the 3-globin CREs (HBG and HBB promoters, and HS1-HS4 enhancers). Colored nodes represent proteins significantly enriched at single or multiple promoter and/or enhancer regions. Size of the circles represents the frequency of interactions.

CAPTURE-3C-seq. 3C Library Preparation and Sequencing. 1 to 5×10⁷ cells were cross-linked with 2 mM EGS (ethylene glycol bis(succinimidyl succinate)) (Thermo-Fisher Scientific) for 45 minutes and 1% formaldehyde for 15 minutes at room temperature. Cross-linking was quenched with 0.25 mM of glycine for 10 minutes at room temperature, followed by two washes with PBS. Cells were resuspended in ice-cold 1 ml of RIPA buffer (10 mM Tris-HCl, 1 mM EDTA, 0.1% sodium deoxycholate, 0.1% SDS, 1% Triton X-100, pH 8.0, freshly added 1 mM DTT, and 1:200 proteinase inhibitor cocktail) and rotated for 15 minutes at 4° C. Cell lysates were centrifuged at 2,300×g for 5 minutes at 4° C. to isolate the nuclei. Nuclei were then resuspended in 500 μl of 1.2× NEBuffer DpnII buffer containing 0.25% SDS and incubated for 10 minutes at 65° C., followed by 1 hour incubation after adding 100 μl of 10% Triton X-100 (final concentration 1.67%). Nuclei were digested using 300 U of DpnII (NEB) on a Thermomixer (Eppendorf) overnight at 37° C. DpnII digestion was quenched by adding 44 μl of 20% SDS (final concentration 1.6%) and vortexed for 20 minutes at 65° C. The digested nuclei were diluted with 2.041 ml of 1.5× T4 ligation buffer (300 μl of 10×NEB T4 ligase buffer, 1.741 ml of ddH₂O, freshly added 1:200 proteinase inhibitor cocktail). SDS was sequestered by adding 700 μl of 10% Triton X-100 and incubating at 37° C. for 1 hour at 400 RPM. Nuclei were then ligated overnight by adding 15 μl of NEB T4 DNA ligase (final concentration 30 weiss U/ml) with rotation overnight at 16° C. The nuclei were collected by centrifuge at 2,300 g for 5 minutes at 4° C., and resuspended in 500 μl 0.5% SDS lysis buffer (0.5% SDS, 10 mM EDTA, 50 mM Tris-HCl, pH 8.0) followed by sonication to shear chromatin fragments to an average size ˜500 bp on the Branson Sonifier 450 ultrasonic processor (10% amplitude, 0.5 second on 1 second off for 30 seconds). Chromatin fragments were centrifuged at 16,100×g for 10 minutes at 4° C. Final concentration 300 mM NaCl was added to the supernatant followed by incubation with 50 μl of MyOne Streptavidin Ti Dynabeads (Thermo-Fisher Scientific) overnight at 4° C. After overnight incubation, the Dynabeads were washed twice with 1 ml of 2% SDS, twice with 1 ml of RIPA buffer with 0.5 M NaCl, twice with 1 ml of LiCl buffer, and twice with 1 ml of TE buffer. The chromatin was resuspended in SDS elution buffer (1% SDS, 10 mM EDTA, 50 mM Tris-HCl, pH 8.0, 0.2 mg/ml proteinase K) followed by reverse cross-linking and proteinase K digestion at 65° C. overnight. The DNA was purified using QIAquick Spin columns (Qiagen). 5 ng of CAPTURE-3C DNA was processed for library generation using the NEBNext ChIP-seq Library Prep Kit (New England Biolabs). Libraries were pooled and 38 bp pair-end sequencing was performed on an Illumina Nextseq500 platform using the 75 bp high output sequencing kit. To determine the specificity of CAPTURE-3C-seq, the inventors performed two control experiments: 1) CAPTURE-3C-seq using the non-targeting sgGal4 control, and 2) CAPTURE-3C-seq using the purified, DpnII-digested genomic DNA (naked gDNA) control. The sgGal4 control was performed in parallel with other target-specific sgRNAs following the same CAPTURE-3C-seq protocol, whereas the gDNA control was performed in the absence of dCas9 affinity purification step to determine the probabilities of ligation of any DpnII-digested DNA fragments due to random collision in the ligation reaction.

CAPTURE-3C-Seq Data Analysis.

To identify significant interactions from sequenced read pairs, the inventors developed a customized data processing pipeline for the mapping of raw reads and statistical analysis. All sequencing reads were mapped to human (hg19) or mouse (mm9) genome assembly. Raw reads from all replicate experiments for each sgRNA sample were merged. Pair-end reads were mapped as single-end reads by using Bowtie2 (Langmead and Salzberg, 2012) with the default parameters to avoid the build-in assumption of the relative positioning of pair-end sequences in the alignment program. Unmapped reads were tested if they contained a DpnII restriction site. The reads with digestion position were trimmed and the longer fragment with length ≥20 bp was collected and remapped. The mapped reads from both procedures were combined and the reads with low mapping quality were removed by using the cutoff of MAPQ ≥30. The mapped reads from pair-end sequencing were then paired. PCR duplicates were removed by discarding the reads with the same positions at both paired ends.

The preprocessed read pairs were used to define the interactions at each sgRNA-targeted (or bait) region to other chromosomal regions. Previous studies of 4C and Capture-C used fixed sizes of sliding window (typically +1 kb of targeted sites) to define the interacting regions (Hughes et al., 2014; van de Werken et al., 2012). However, the peaks of local read pairs (or self-ligations) are different from each experiment and skewness of peaks can be observed from the sgRNA-targeted regions. Hence, fixed window sizes with 2 kb would have hard cutoff of bait regions and may lead to inaccurate positioning of bait regions. Therefore, the inventors defined the bait region as the local peaks surrounding the sgRNA target site by using MACS2 with default parameters (Zhang et al., 2008). The read pairs located within the bait region were considered as self-ligated reads and filtered. After preprocessing and filtering, the resulting data is a list of count numbers of read pairs from the bait region to any chromosomal regions. A pair of reads that located within two different regions is considered an interaction. The inventors then applied separate background models to calculate the significance for intra- and inter-chromosomal interactions.

Intra-chromosomal model:

To understand the statistical significance of enrichment for x_{d}(i) that denotes the interaction numbers from the bait region to the chromosomal region i with distance d*l, the inventors need to know the bias/noise background of x_{d}(i). Here d is the indicator of the region that is with distance of d*l to the bait region, where 1 is the size of bait region. The inventors used interaction values X_{d} of any two regions in the same chromosome as the background (excluding the bait region). The inventors found (1) the means/medians of X_{d} were decreased when distances increased; (2) the mean and variance showed proportional relationship revealed by linear regression analysis. To better fit the underlying observations, the Bayesian mixture model was used to describe the interaction background and presented multiple models for different distance d. The count of interactions X_{d} is assumed to have been drawn from a Poisson distribution with mean λ_{d}, which follows a Gamma distribution with parameters α_{d} and β_{d}. e.g X_{d}˜Poisson(λ_{d}), λ_{d}˜Gamma(α_{d}, β_{d}), yielding:

$$Pr ( {X}_{d} \vert {α}_{d} , {β}_{d} ) = {\int }_{0}^{∞} Pr ( {X}_{d} ∼ Poisson ( {λ}_{d} ) ) Pr ( {λ}_{d} ∼ Gamma ( {α}_{d} , {β}_{d} ) ) d {λ}_{d} = \frac{{β}_{d}^{{α}_{d}} Γ ( {α}_{d} + {X}_{d} )}{{( {β}_{d} + 1 )}^{{α}_{d} + {X}_{d}} Γ ( {α}_{d} ) {X}_{d} !}$$

Thus, the user can get X_{d} follows a negative binomial distribution with parameters α_{d} and

$$\frac{{β}_{d}}{{β}_{d} + 1} .$$

A Maximum Likelihood Estimator (MLE) was used to estimate the parameters α_{d} and β_{d}. Since negative binomial distribution has a closed form of expected value, a great practical advantage can be achieved to estimate parameters by using simple mean and variance. Thus, X_{d} models the random collision frequency between any two chromosomal regions (with distance of d). Thus, the user can therefore calculate P values by using negative binomial distribution to reflect the significance of x_{d}(i) as P_{d}(i)=P(X_{d}<x_{d}(i)). Specifically, the bigger P_{d}(i) indicates lower possibility of random collisions that are bigger than X_{d}(i), suggesting higher confidence of interactions between the bait region and the chromosomal region i. Instead of calculating P values, the Bayes factor (BF) was used to compare the hypothesis H₀ that specific interactions have occurred between the bait region and a given chromosomal region (Pr(H₀|x_{d}(i))=P(X_{d}<x_{d}(i)), e.g. the probability that random collisions are less than observed interaction x_{d}(i)), against the alternative hypothesis H₁, representing no interactions between them. The BF is defined as

$$BF = \frac{Pr ( {x}_{d} ( i ) \vert {H}_{0} )}{Pr ( {x}_{d} ( i ) \vert {H}_{1} )} = \frac{Pr ( {H}_{0} \vert {x}_{d} ( i ) )}{Pr ( {H}_{1} \vert {x}_{d} ( i ) )} \frac{Pr ( {H}_{1} )}{Pr ( {H}_{0} )} ,$$

a strength measure for comparing two hypotheses, which provides a natural way to consider the uncertainty in hypothesis testing and controlling false discovery rate (FDR). Here, the prior odds

$$\frac{Pr ( {H}_{1} )}{Pr ( {H}_{0} )}$$

were assigned as 0.001, indicating that random collision bigger than true interactions is a rare event. According to the scale for BF, 3≤BF<20 is considered ‘positive’ and 20≤BF is considered ‘strong’ evidence of supporting H₀ (Kass and Raftery, 1995). Here, the inventors considered paired regions with BF of interactions more than 20 as the ‘high-confidence interactions’. The inventors set up 11 different models for different distance d, including 10 models for paired regions with distances ranged from 1*l to 10*l and one for paired regions with distances bigger than 10*l, where l is the size of the bait region.

Inter-Chromosomal Model:

To test the significance of interactions between the bait region to the interacting regions on a different chromosome, the inventors developed the background model by using the random collisions among inter-chromosomal region pairs (regions located on different chromosomes). Specifically, the inventors first extended the bait region to 1 Mb and split all chromosomes into 1 Mb regions. For a region j of other chromosomes (excluding chr11), the inventors counted the numbers from the bait region to region j. The inventors randomly selected 1000 regions from chr11 and counted interactions from them to region j as the background (negative binomial distribution). Similar to the intra-chromosomal model, the inventors also used the Bayes factor (BF) to test if interactions from the bait region and other regions were significant. All scripts are tested on Linux operating system and available on request.

Comparison of chromatin interactions defined by CAPTURE-3C-seq, 4C, 5C, ChIA-PET and Hi-C. RNAPII and CTCF ChIA-PET (GSM970213 and GSM970216), UMI-4C (GSM2037371), 5C (GSM970500), DNase Hi-C (GSM1370434 and GSM1370436), and in situ Hi-C data (GSM1551618) were downloaded from GEO (Table S1). The raw reads from all samples were mapped by Bowtie2 using the same parameters as in CAPTURE-3C-seq. The unique read pairs with one end in bait region (PETs) were collected. The inventors then calculated the normalized PETs of a bait region as

$$\frac{PETs \cdot {10}^{9}}{Bait\_Length \cdot Total\_reads .} ,$$

which represents the on-target enrichment as the number of PETs per kilobases of bait region per million mapped reads. The unique PETs were defined as pair-end sequence tags with distinct genomic locations at one or both sides of the pair-end reads.

CRISPR Imaging of Human Telomeres. CRISPR imaging of human telomeres was performed as described (Chen et al., 2013). Briefly, human MCF7 cells were transduced with lentiviruses expressing a dCas9-EGFP fusion protein driven by a TRE3G promoter and the Tet-on-3G trans-activator protein. After confirming the expression of the dCas9-EGFP fusion protein by induction with doxycycline (100 ng/ml), the cells were transduced with lentiviruses expressing the telomere-specific sgRNA (sgTelomere) in an 8-well chambered coverglass. The nuclear location of dCas9-EGFP was determined on a 2-photon fluorescence microscope (Zeiss LSM780 Inverted) with 40× and 60× objective lens. The images were acquired and analyzed on the ZEN software (Zeiss).

RNA-seq and qRT-PCR Analysis. Total RNA was isolated using RNeasy Plus Mini Kit (Qiagen) following manufacturer's protocol. RNA-seq library was prepared using the Truseq v2 LT Sample Prep Kit (Illumina) or the Ovation RNA-seq system (NuGEN). Sequencing reads from all RNA-seq experiments were aligned to human (hg19) reference genome by TopHat v2.0.13 (Trapnell et al., 2009) with the parameters: --solexaquals --no-novel-juncs. Quantitative RT-PCR (qRT-PCR) was performed using the iQ SYBR Green Supermix (Bio-Rad). Primer sequences are listed in Table 2.

ChIP-seq Analysis. ChIP-seq was performed as described (Huang et al., 2016) using the antibodies for BRD4 (A301-985A, Bethyl, lot: A301-985A-1), RNAPII (MMS-126R, Covance, lot: D12LF03144) and H3K27ac (ab4729, Abcam) in K562 erythroid cells treated with DMSO (control), or 1 μM of JQ1 for 6 hours. Antibodies for NUP98 (2598, Cell Signaling Technology, lot: 4) or NUP153 (906201, BioLegend, lot: B215613) were used. Cross-linked K562 chromatin was sonicated in RIPA 0 buffer (10 mM Tris-HCl, 1 mM EDTA, 0.1% sodium deoxycholate, 0.1% SDS, 1% Triton X-100, 0.25% Sarkosyl, pH 8.0) to 200-500 bp. Final concentration 150 mM NaCl was added to the chromatin and antibody mixture before incubation overnight at 4° C. ChIP-seq libraries were generated using NEBNext ChIP-seq Library Prep Master Mix following the manufacturer's protocol (New England Biolabs), and sequenced on an Illumina NextSeq500 system using the 75 bp high output sequencing kit. ChIP-seq raw reads were aligned to the hg19 or mm9 genome assembly using Bowtie (Langmead et al., 2009) with the default parameters. Only tags that uniquely mapped to the genome were used for further analysis. ChIP-seq peaks were identified using MACS (Zhang et al., 2008). Gene ontology (GO) analysis was performed using GREAT (McLean et al., 2010).

ATAC-seq Analysis. 5×10⁴ cells were washed twice in PBS and resuspended in 500 μl lysis buffer (10 mM Tris-HCl, 10 mM NaCl, 3 mM MgCl, 0.1% NP-40, pH 7.4). Nuclei were harvested by centrifuge at 500×g for 10 minutes at 4° C. Nuclei were suspended in 50 μl of tagmentation mix (10 mM TAPS (Sigma), 5 mM MgCl, pH 8.0 and 2.5 μl Tn5) and incubated at 37° C. for 30 minutes. Tagmentation reaction was terminated by incubating nuclei at room temperature for 2 minutes followed by incubation at 55° C. for 7 minutes after adding 10 μl of 0.2% SDS. Tn5 tranposase-tagged DNA was purified using QIAquick MinElute PCR Purification kit (Qiagen), amplified using KAPA HiFi Hotstart PCR Kit (KAPA), and sequenced on an Illumina Nextseq500 system using the 75 bp high output sequencing kit. ATAC-seq raw reads were trimmed to remove adaptor sequence and aligned to hg19 or mm9 genome assembly using Bowtie2 (Langmead et al., 2009) with k=1 and m=1. Only tags that uniquely mapped to the genome were used for further analysis.

Flow Cytometry. Human erythroid cell differentiation was analyzed by flow cytometry using FACSCanto. Live cells were identified and gated by exclusion of 7-amino-actinomycin D (7-AAD; BD Pharmingen). The cells were analyzed for expression of cell surface receptors with antibodies specific for CD71 and CD235a conjugated to phycoerythrin (PE) and fluorescein isothiocyanate (FITC), respectively. Data were analyzed using FlowJo software (Ashland, Oreg.).

Cytospin. Cytospin preparations from cells at various stages of erythroid differentiation were stained with May-Grunwald-Giemsa as described previously (Xu et al., 2011).

CRISPR/Cas9-Mediated Knockout of Cis-Regulatory Elements. The CRISPR/Cas9 system was used to introduce deletion mutations of the cis-regulatory elements in K562 cells following published protocols (Cong et al., 2013; Mali et al., 2013). Briefly, sequence-specific sgRNAs for site-specific cleavage of genomic targets were designed following described guidelines, and sequences were selected to minimize off-target cleavage based on publicly available filtering tools (http://crispr.mit.edu/). Oligonucleotides were annealed in the following reaction: 10 μM guide sequence oligo, 10 μM reverse complement oligo, T4 ligation buffer (1×), and 5 U of T4 polynucleotide kinase with the cycling parameters of 37° C. for 30 minutes; 95° C. for 5 minutes and then ramp down to 25° C. at 5° C./minutes. The annealed oligos were cloned into the pSpCas9(BB) (pX458) vector (Addgene #48138) using a Golden Gate Assembly strategy including: 100 ng of circular pX458 plasmid, 0.2 LM annealed oligos, 2.1 buffer (1×) (New England Biolabs), 20 U of BbsI restriction enzyme, 0.2 mM ATP, 0.1 mg/ml BSA, and 750 U of T4 DNA ligase (New England Biolabs) with the cycling parameters of 20 cycles of 37° C. for 5 minutes, 20° C. for 5 minutes; followed by 80° C. incubation for 20 minutes. To induce deletions of candidate regulatory DNA regions, two CRISPR/Cas9 constructs were co-transfected into K562 cells by nucleofection using the ECM 830 Square Wave Electroporation System (Harvard Apparatus). Each construct was directed to flanking the target genomic regions. To enrich for deletion, the top 1-5% of GFP-positive cells were FACS sorted 48-72 hours post-transfection and plated in 96-well plates. Single-cell-derived clones were isolated and screened for CRISPR-mediated deletion of target genomic sequences. PCR amplicons were subcloned and analyzed by Sanger DNA sequencing to confirm non-homologous end-joining (NHEJ)-mediated repair upon double-strand break formation. The positive single-cell-derived clones containing deletion of the targeted sequences were expanded and processed for analysis.

Generation of Tetracycline-Inducible dCas9 Knock-in ESCs. Site-specific knock-in of tetracycline-inducible FLAG-biotin-acceptor-site (FB)-tagged dCas9-EGFP and BirA transgenes was generated through flippase (FLPe)-mediated recombination (Beard et al., 2006). Briefly, KH2 mouse embryonic stem cells (ESCs) harboring a targeted M2rtTA tetracycline-responsive trans-activator in the Rosa26 locus and a modified Collal locus with an frt site and ATG-less hygromycin resistance gene were used. A targeting construct pBS3.1-FB-dCas9-IRES-BirA containing the PGK promoter, an frt site, a tetracycline-inducible minimal CMV promoter, the FB-dCas9-EGFP-IRES-BirA transgenes, and an ATG initiation codon was co-electroporated with the pCAGGS-FLPe-puro into KH2 ESCs at 500V and 25 μF using a Gene Pulser II (Bio-Rad). The cells were selected with hygromycin (140 μg/ml) after 24 hours. The positive clones were expanded and analyzed by genotyping PCR. The correctly targeted ESCs were cultured in the absence or presence of doxycycline (0.1-1 μg/ml) for 48 hours and harvested for CAPTURE experiments.

Quantification and Statistical Analysis. Statistical details including N, mean and statistical significance values are indicated in the text, figure legends, or Method Details. Error bars in the experiments represent standard error of the mean (SEM) from either independent experiments or independent samples. All statistical analyses were performed using GraphPad Prism, and the detailed information about statistical methods is specified in figure legends or Methods Details.

Data and Software Availability. All raw and processed RNA-seq, ChIP-seq, CAPTURE-ChIP-seq, CAPTURE-3C-seq and ATAC-seq data are available in the Gene Expression Omnibus (GEO): GSE88817.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Beard, C., Hochedlinger, K., Plath, K., Wutz, A., and Jaenisch, R. (2006). Efficient method to generate single-copy transgenic mice by site-specific integration in embryonic stem cells. Genesis (New York, N.Y.: 2000) 44, 23-28.
Capelson, M., Liang, Y., Schulte, R., Mair, W., Wagner, U., and Hetzer, M. W. (2010). Chromatin-bound nuclear pore components regulate gene expression in higher eukaryotes. Cell 140, 372-383.
Chen, B., Gilbert, L. A., Cimini, B. A., Schnitzbauer, J., Zhang, W., Li, G. W., Park, J., Blackburn, E. H., Weissman, J. S., Qi, L. S., et al. (2013). Dynamic imaging of genomic loci in living human cells by an optimized CRISPR/Cas system. Cell 155, 1479-1491.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science (New York, N.Y.) 339, 819-823.
Consortium, T. E. P. (2012). An integrated encyclopedia of DNA elements in the human genome. Nature 489, 57-74.
Dejardin, J., and Kingston, R. E. (2009). Purification of proteins associated with specific genomic Loci. Cell 136, 175-186.
Dekker, J., Rippe, K., Dekker, M., and Kleckner, N. (2002). Capturing chromosome conformation. Science (New York, N.Y.) 295, 1306-1311.
Deng, W., Lee, J., Wang, H., Miller, J., Reik, A., Gregory, P. D., Dean, A., and Blobel, G. A. (2012). Controlling long-range genomic interactions at a native locus by targeted tethering of a looping factor. Cell 149, 1233-1244.
Deng, W., Rupon, J. W., Krivega, I., Breda, L., Motta, I., Jahn, K. S., Reik, A., Gregory, P. D., Rivella, S., Dean, A., et al. (2014). Reactivation of developmentally silenced globin genes by forced chromatin looping. Cell 158, 849-860.
Dixon, J. R., Selvaraj, S., Yue, F., Kim, A., Li, Y., Shen, Y., Hu, M., Liu, J. S., and Ren, B. (2012). Topological domains in mammalian genomes identified by analysis of chromatin interactions. Nature 485, 376-380.
Dostie, J., Richmond, T. A., Arnaout, R. A., Selzer, R. R., Lee, W. L., Honan, T. A., Rubio, E. D., Krumm, A., Lamb, J., Nusbaum, C., et al. (2006). Chromosome Conformation Capture Carbon Copy (5C): a massively parallel solution for mapping interactions between genomic elements. Genome research 16, 1299-1309.
Elias, J. E., and Gygi, S. P. (2007). Target-decoy search strategy for increased confidence in large-scale protein identifications by mass spectrometry. Nature methods 4, 207-214.
Filippakopoulos, P., Qi, J., Picaud, S., Shen, Y., Smith, W. B., Fedorov, O., Morse, E. M., Keates, T., Hickman, T. T., Felletar, I., et al. (2010). Selective inhibition of BET bromodomains. Nature 468, 1067-1073.
Fraser, P., Pruzina, S., Antoniou, M., and Grosveld, F. (1993). Each hypersensitive site of the human beta-globin locus control region confers a different developmental pattern of expression on the globin genes. Genes & development 7, 106-113.
Fujita, T., Asano, Y., Ohtsuka, J., Takada, Y., Saito, K., Ohki, R., and Fujii, H. (2013). Identification of telomere-associated molecules by engineered DNA-binding molecule-mediated chromatin immunoprecipitation (enChIP). Scientific reports 3, 3171.
Fujita, T., and Fujii, H. (2013). Efficient isolation of specific genomic regions and identification of associated proteins by engineered DNA-binding molecule-mediated chromatin immunoprecipitation (enChIP) using CRISPR. Biochemical and biophysical research communications 439, 132-136.
Fullwood, M. J., Liu, M. H., Pan, Y. F., Liu, J., Xu, H., Mohamed, Y. B., Orlov, Y. L., Velkov, S., Ho, A., Mei, P. H., et al. (2009). An oestrogen-receptor-alpha-bound human chromatin interactome. Nature 462, 58-64.
Hardison, R., Slightom, J. L., Gumucio, D. L., Goodman, M., Stojanovic, N., and Miller, W. (1997). Locus control regions of mammalian beta-globin gene clusters: combining phylogenetic analyses and experimental results to gain functional insights. Gene 205, 73-94.
Huang, J., Liu, X., Li, D., Shao, Z., Cao, H., Zhang, Y., Trompouki, E., Bowman, T. V., Zon, L. I., Yuan, G. C., et al. (2016). Dynamic Control of Enhancer Repertoires Drives Lineage and Stage-Specific Transcription during Hematopoiesis. Developmental Cell 36, 9-23.
Hughes, J. R., Roberts, N., McGowan, S., Hay, D., Giannoulatou, E., Lynch, M., De Gobbi, M., Taylor, S., Gibbons, R., and Higgs, D. R. (2014). Analysis of hundreds of cis-regulatory landscapes at high resolution in a single, high-throughput experiment. Nature genetics 46, 205-212.
Ibarra, A., Benner, C., Tyagi, S., Cool, J., and Hetzer, M. W. (2016). Nucleoporin-mediated regulation of cell identity genes. Genes & development 30, 2253-2258.
Kagey, M. H., Newman, J. J., Bilodeau, S., Zhan, Y., Orlando, D. A., van Berkum, N. L., Ebmeier, C. C., Goossens, J., Rahl, P. B., Levine, S. S., et al. (2010). Mediator and cohesin connect gene expression and chromatin architecture. Nature 467, 430-435.
Kalverda, B., Pickersgill, H., Shloma, V. V., and Fornerod, M. (2010). Nucleoporins directly stimulate expression of developmental and cell-cycle genes inside the nucleoplasm. Cell 140, 360-371.
Kass, R. E., and Raftery, A. E. (1995). Bayes Factors. Journal of the American Statistical Association 90, 773-795.
Kim, J., Cantor, A. B., Orkin, S. H., and Wang, J. (2009a). Use of in vivo biotinylation to study protein-protein and protein-DNA interactions in mouse embryonic stem cells. Nature protocols 4, 506-517.
Kim, S. I., Bultman, S. J., Kiefer, C. M., Dean, A., and Bresnick, E. H. (2009b). BRG1 requirement for long-range interaction of a locus control region with a downstream promoter. Proceedings of the National Academy of Sciences of the United States of America 106, 2259-2264.
Langmead, B., and Salzberg, S. L. (2012). Fast gapped-read alignment with Bowtie 2. Nature methods 9, 357-359.
Langmead, B., Trapnell, C., Pop, M., and Salzberg, S. L. (2009). Ultrafast and memory-efficient alignment of short DNA sequences to the human genome. Genome biology 10, R25.
Lewis, K. A., and Wuttke, D. S. (2012). Telomerase and telomere-associated proteins: structural insights into mechanism and evolution. Structure (London, England: 1993) 20, 28-39.
Li, G., Ruan, X., Auerbach, R. K., Sandhu, K. S., Zheng, M., Wang, P., Poh, H. M., Goh, Y., Lim, J., Zhang, J., et al. (2012). Extensive promoter-centered chromatin interactions provide a topological basis for transcription regulation. Cell 148, 84-98.
Lieberman-Aiden, E., van Berkum, N. L., Williams, L., Imakaev, M., Ragoczy, T., Telling, A., Amit, I., Lajoie, B. R., Sabo, P. J., Dorschner, M. O., et al. (2009). Comprehensive mapping of long-range interactions reveals folding principles of the human genome. Science (New York, N.Y.) 326, 289-293.
Ma, W., Ay, F., Lee, C., Gulsoy, G., Deng, X., Cook, S., Hesson, J., Cavanaugh, C., Ware, C. B., Krumm, A., et al. (2015). Fine-scale chromatin interaction maps reveal the cis-regulatory landscape of human lincRNA genes. Nature methods 12, 71-78.
Mali, P., Yang, L., Esvelt, K. M., Aach, J., Guell, M., DiCarlo, J. E., Norville, J. E., and Church, G. M. (2013). RNA-guided human genome engineering via Cas9. Science (New York, N.Y.) 339, 823-826.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nature biotechnology 28, 495-501.
Miccio, A., and Blobel, G. A. (2010). Role of the GATA-1/FOG-1/NuRD pathway in the expression of human beta-like globin genes. Molecular and cellular biology 30, 3460-3470.
Morley, B. J., Abbott, C. A., Sharpe, J. A., Lida, J., Chan-Thomas, P. S., and Wood, W. G. (1992). A single beta-globin locus control region element (5′ hypersensitive site 2) is sufficient for developmental regulation of human globin genes in transgenic mice. Molecular and cellular biology 12, 2057-2066.
Naumova, N., Imakaev, M., Fudenberg, G., Zhan, Y., Lajoie, B. R., Mirny, L. A., and Dekker, J. (2013). Organization of the mitotic chromosome. Science (New York, N.Y.) 342, 948-953.
Navas, P. A., Peterson, K. R., Li, Q., Skarpidi, E., Rohde, A., Shaw, S. E., Clegg, C. H., Asano, H., and Stamatoyannopoulos, G. (1998). Developmental specificity of the interaction between the locus control region and embryonic or fetal globin genes in transgenic mice with an HS3 core deletion. Molecular and cellular biology 18, 4188-4196.
Osborne, C. S., Chakalova, L., Brown, K. E., Carter, D., Horton, A., Debrand, E., Goyenechea, B., Mitchell, J. A., Lopes, S., Reik, W., et al. (2004). Active genes dynamically colocalize to shared sites of ongoing transcription. Nature genetics 36, 1065-1071.
Palstra, R. J., Tolhuis, B., Splinter, E., Nijmeijer, R., Grosveld, F., and de Laat, W. (2003). The beta-globin nuclear compartment in development and erythroid differentiation. Nature genetics 35, 190-194.
Peterson, K. R., Navas, P. A., Li, Q., and Stamatoyannopoulos, G. (1998). LCR-dependent gene expression in beta-globin YAC transgenics: detailed structural studies validate functional analysis even in the presence of fragmented YACs. Hum Mol Genet 7, 2079-2088.
Rao, S. S., Huntley, M. H., Durand, N. C., Stamenova, E. K., Bochkov, I. D., Robinson, J. T., Sanborn, A. L., Machol, I., Omer, A. D., Lander, E. S., et al. (2014). A 3D map of the human genome at kilobase resolution reveals principles of chromatin looping. Cell 159, 1665-1680.
Sankaran, V. G., Xu, J., Byron, R., Greisman, H. A., Fisher, C., Weatherall, D. J., Sabath, D. E., Groudine, M., Orkin, S. H., Premawardhena, A., et al. (2011). A functional element necessary for fetal hemoglobin silencing. The New England journal of medicine 365, 807-814.
Schatz, P. J. (1993). Use of peptide libraries to map the substrate specificity of a peptide-modifying enzyme: a 13 residue consensus peptide specifies biotinylation in Escherichia coli. Bio/technology (Nature Publishing Company) 11, 1138-1143.
Schwartzman, O., Mukamel, Z., Oded-Elkayam, N., Olivares-Chauvet, P., Lubling, Y., Landan, G., Izraeli, S., and Tanay, A. (2016). UMI-4C for quantitative and targeted chromosomal contact profiling. Nature methods 13, 685-691.
Shao, Z., Zhang, Y., Yuan, G. C., Orkin, S. H., and Waxman, D. J. (2012). MAnorm: a robust model for quantitative comparison of ChIP-Seq data sets. Genome biology 13, R16.
Simonis, M., Klous, P., Splinter, E., Moshkin, Y., Willemsen, R., de Wit, E., van Steensel, B., and de Laat, W. (2006). Nuclear organization of active and inactive chromatin domains uncovered by chromosome conformation capture-on-chip (4C). Nature genetics 38, 1348-1354.
Stonestrom, A. J., Hsu, S. C., Jahn, K. S., Huang, P., Keller, C. A., Giardine, B. M., Kadauke, S., Campbell, A. E., Evans, P., Hardison, R. C., et al. (2015). Functions of BET proteins in erythroid gene expression. Blood 125, 2825-2834.
Thurman, R. E., Rynes, E., Humbert, R., Vierstra, J., Maurano, M. T., Haugen, E., Sheffield, N. C., Stergachis, A. B., Wang, H., Vernot, B., et al. (2012). The accessible chromatin landscape of the human genome. Nature 489, 75-82.
Tolhuis, B., Palstra, R. J., Splinter, E., Grosveld, F., and de Laat, W. (2002). Looping and interaction between hypersensitive sites in the active beta-globin locus. Molecular cell 10, 1453-1465.
Trapnell, C., Pachter, L., and Salzberg, S. L. (2009). TopHat: discovering splice junctions with RNA-Seq. Bioinformatics (Oxford, England) 25, 1105-1111.
van de Werken, H. J., Landan, G., Holwerda, S. J., Hoichman, M., Klous, P., Chachik, R., Splinter, E., Valdes-Quezada, C., Oz, Y., Bouwman, B. A., et al. (2012). Robust 4C-seq data analysis to screen for regulatory DNA interactions. Nature methods 9, 969-972.
Waldrip, Z. J., Byrum, S. D., Storey, A. J., Gao, J., Byrd, A. K., Mackintosh, S. G., Wahls, W. P., Taverna, S. D., Raney, K. D., and Tackett, A. J. (2014). A CRISPR-based approach for proteomic analysis of a single genomic locus. Epigenetics 9, 1207-1211.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Xu, J., Bauer, D. E., Kerenyi, M. A., Vo, T. D., Hou, S., Hsu, Y. J., Yao, H., Trowbridge, J. J., Mandel, G., and Orkin, S. H. (2013). Corepressor-dependent silencing of fetal hemoglobin expression by BCL11A. Proceedings of the National Academy of Sciences of the United States of America 110, 6518-6523.
Xu, J., Peng, C., Sankaran, V. G., Shao, Z., Esrick, E. B., Chong, B. G., Ippolito, G. C., Fujiwara, Y., Ebert, B. L., Tucker, P. W., et al. (2011). Correction of sickle cell disease in adult mice by interference with fetal hemoglobin silencing. Science (New York, N.Y.) 334, 993-996.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome biology 9, R137.
Zhao, Z., Tavoosidana, G., Sjolinder, M., Gondor, A., Mariano, P., Wang, S., Kanduri, C., Lezcano, M., Sandhu, K. S., Singh, U., et al. (2006). Circular chromosome conformation capture (4C) uncovers extensive networks of epigenetically regulated intra- and interchromosomal interactions. Nature genetics 38, 1341-1347.
Zhou, F., Lu, Y., Ficarro, S. B., Adelmant, G., Jiang, W., Luckey, C. J., and Marto, J. A. (2013). Genome-scale proteome quantification by DEEP SEQ mass spectrometry. Nature communications 4, 2171.

What is claimed is:

1. A method for detecting or isolating one or more specific genomic target regions and molecules interacting therewith comprising:
contacting a recombinant nuclease-deficient Cas9 fusion protein (dCas9) modified to comprise a biotinylation sequence and one or more sequence-specific guide RNAs, with one or more specific genomic DNA targets in cells to form a CRISPR complex; and
detecting or isolating the CRISPR complex with a streptavidin or an avidin to detect or isolate the one or more specific genomic target regions and molecules in the CRISPR complex.

2. The method of claim 1, further comprising at least one of: (1) fragmenting a genomic DNA in a cell under conditions in which the genomic DNA and molecules interacting therewith are maintained in the CRISPR complex, isolating the CRISPR complex after fragmentation of the genomic DNA; (2) identifying one or more of proteins, peptides, nucleic acids, genomic DNA, or molecules in the CRISPR complex; or (3) detecting the CRISPR complex in situ with the streptavidin or avidin bound to a detectable label.

3. The method of claim 1, wherein the one or more sequence-specific guide RNAs are programmable sequence-specific guide RNAs.

4. The method of claim 1, wherein the recombinant nuclease-deficient Cas9 fusion protein has been: (1) modified to comprise a biotinylation sequence that is biotinylatable in vivo; (2) further comprises an isolatable peptide tag at the N- or C-terminus, or other regions of the dCas9 protein; or (3) is biotinylated in vivo by BirA enzyme or endogenous biotin ligases in prokaryotic cells, eukaryotic cells, or both.

5. The method of claim 4, wherein the isolatable peptide tags are selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both.

6. The method of claim 1, wherein the recombinant nuclease-deficient dCas9 fusion protein is bound with the streptavidin or avidin that has been conjugated to a detectable label selected from at least one of an electrochemiluminescence label, an enzyme label, a fluorophore, a latex particle, a magnetic particle, a radioactive element, a phosphorescent dye, a dye, a gold, silver, or selenium particle, or a ruthenium or osmium metal chelate, wherein the streptavidin or avidin is optionally bound to a solid support, a chip, a substrate, a column, a well, or beads.

7. The method of claim 1, further comprising performing a chemical treatment that maintains the interaction of the genomic DNA and molecules interacting therewith in the CRISPR complex.

8. The method of claim 1, wherein the recombinant nuclease-deficient Cas9 fusion protein is SEQ ID NO:334.

9. The method of claim 1, further comprising expressing in the cells a biotin ligase capable of biotinylating the recombinant nuclease-deficient Cas9 fusion protein.

10. The method of claim 1, further comprising at least one of: (1) capturing in situ one or more locus-specific chromatin interactions by biotinylated dCas9 fusion protein; (2) using biotinylated dCas9-mediated capture of the binding cluster at or around the sequence-specific guide RNA; (3) identifying cis-regulatory elements (CRE)-associated protein complexes to identify proteins or nucleic acids of the CRISPR complex; (4) using the CRISPR complex for CRISPR affinity purification in situ of regulatory elements (CAPTURE)-proteomics to identify known and new regulators of at least one of genes, promoters, or enhancers by: cross-linking the CRISPR complex, fragmenting the complex, dCas9 fusion protein affinity purification, and sequencing the nucleic acids isolated therewith, western blot, or peptide digestion with multiplex identification by proteomic profiling; (5) using CAPTURE-3C-seq to identify locus-specific long-range DNA interactions by crosslinking of the CRISPR complex, enzymatic digestion of nucleic acids, proximity ligation of the nucleic acids, fragmentation of the genomic DNA, dCas9 fusion protein affinity purification, and pair-end sequencing to identify tethered long-range interactions; (6) using biotinylated dCas9-mediated in situ capture of a disease-associated cis-regulatory elements (CRE) to measure cis-transcription factors, RNA complexes, and long-range DNA interactions that contribute to the disease phenotypes; (7) detecting the CRISPR complex in situ; (8) using multiplexed CAPTURE of developmentally regulated super-enhancers during differentiation; (9) identifying nucleic acids, peptides, proteins, by at least one of mass spectrometry (MS)-based proteomics, MS-MS, MALDI, MALDI-TOF, multiplex proteomic identification, immunoblot, ELISA, nucleotide sequence analysis, microarray analysis, or PCR; or (10) using multiplexed CAPTURE using 2, 3, 4, 5, 6, 7, 8, 9, 10, 15, 20, 25, 30, 35, 40, 45, 50, 60, 70, 75, 80, 90, 100, 200, 300, 400, 500, 600, 700, 800, 900, 1,000, 2,000, 3,000, 4000, 5,000, 6,000, 7,000, 8,000, 9,000, 10,000, or more sgRNAs in a pool to target multiple genomic regions, including multiple cis-elements at the same enhancer cluster or multiple independent enhancers.

11. The method of claim 10, wherein the enzymatic digestion is by at least one of AatI, AatII, AauI, Acc113I, Acc16I, Acc65I, AccB1I, AccB7I, AccBSI, AccI, AccII, AccIII, AceIII, AciI, AclI, AclNI, AclWI, AcsI, AcyI, AdeI, AfaI, AfeI, AflII, AflIII, AgeI, AhaIII, AhdI, AluI, Alw21I, Alw26I, Alw44I, AlwI, AlwNI, Ama87I, AocI, Aor51HI, ApaBI, Apal, ApaLI, ApoI, AscI, Asel, AsiAI, AsnI, Asp700I, Asp718I, AspEI, AspHI, AspI, AspLEI, AspS9I, AsuC2I, AsuHPI, AsuI, AsuII, AsuNHI, AvaI, AvaII, AvaIII, AviII, AvrII, AxyI, BaeI, BalI, BamHI, BanI, BanII, BanIII, BbeI, BbiII, BbrPI, BbsI, BbuI, Bbv12I, BbvCI, BbvI, BbvII, BccI, Bce83I, BcefI, BcgI, BciVI, BclI, BcnI, BcoI, BcuI, BetI, BfaI, BfiI, BfmI, BfrI, BglI, BglII, BinI, BlnI, BlpI, Bme18I, BmgI, BmrI, BmyI, BpiI, BplI, BpmI, Bpu10I, Bpu1102I, Bpu14I, BpuAI, Bsa29I, BsaAI, BsaBI, BsaHI, BsaI, BsaJI, BsaMI, BsaOI, BsaWI, BsaXI, BsbI, Bsc4I, BscBI, BscCI, BscFI, BscGI, BscI, Bse118I, Bse1I, Bse21I, Bse3DI, Bse8I, BseAI, BseCI, BseDI, BseGI, BseLI, BseMII, BseNI, BsePI, BseRI, BseX3I, BsgI, Bsh1236I, Bsh1285I, Bsh1365I, BshI, BshNI, BsiBI, BsiCI, BsiEI, BsiHKAI, BsiI, BsiLI, BsiMI, BsiQI, BsiSI, BsiWI, BsiXI, BsiYI, BsiZI, BslI, BsmAI, BsmBI, BsmFI, BsmI, BsoBI, Bsp106I, Bsp19I, Bsp120I, Bsp1286I, Bsp13I, Bsp1407I, Bsp143I, Bsp143II, Bsp1720I, Bsp19I, Bsp24I, Bsp68I, BspA2I, BspCI, BspDI, BspEI, BspGI, BspHI, BspLI, BspLU11I, BspMI, BspMII, BspTI, BspXI, BsrBI, BsrBRI, BsrDI, BsrFI, BsrGI, BsrI, BsrSI, BssAI, BssHII, BssKI, BssNAI, BssSI, BssT1I, Bst1107I, Bst2BI, Bst2UI, Bst4CI, Bst71I, Bst98I, BstACI, BstAPI, BstBAI, BstBI, BstDEI, BstDSI, BstEII, BstFSI, BstH2I, BstHPI, BstMCI, BstNI, BstNSI, BstOI, BstPI, BstSFI, BstSNI, BstUI, BstX2I, BstXI, BstYI, BstZ17, BstZI, Bsu15I, Bsu36I, Bsu6I, BsuRI, BtgI, BtsI, Cac8I, CauII, CbiI, CciNI, CelII, CfoI, Cfr10I, Cfr13I, Cfr42I, Cfr9I, CfrI, CjeI, CjePI, ClaI, CpoI, Csp45I, Csp6I, CspI, CviJI, CviRI, CvnI, DdeI, DpnI, DpnII, DraI, DraII, DraIII, DrdI, DrdII, DsaI, DseDI, EaeI, EagI, Eam1104I, Eaml 11051, EarI, EciI, Ec136II, EclHKI, EclXI, Eco105I, Eco130I, Eco147I, Eco24I, Eco255I, Eco31I, Eco32I, Eco47I, Eco47III, Eco52I, Eco57I, Eco64I, Eco72I, Eco81I, Eco88I, Eco91I, EcoICRI, EcoNI, EcoO109I, EcoO65I, EcoRI, EcoRII, EcoRV, EcoT14I, EcoT22I, EcoT38I, EgeI, EheI, ErhI, Esp1396I, Esp3I, EspI, FauI, FauNDI, FbaI, FinI, Fnu4HI, FnuDUII, FokI, FriOI, FseI, Fsp4HI, FspI, GdiII, GsuI, HaeI, HaeII, HaeIII, HaeIV, HapII, HgaI, HgiAI, HgiCI, HgiEI, HgiEII, HgiJII, HhaI, Hin1I, Hin2I, Hin4I, Hin6I, HincII, HindII, HindIII, HinfI, HinP1I, HpaI, HpaII, HphI, Hsp92I, Hsp92II, HspAI, ItaI, KasI, Kpn2I, KpnI, Ksp22I, Ksp632I, KspAI, KspI, Kzo9I, LspI, MaeI, MaeII, MaeIII, MamI, MbiI, MboI, MboII, McrI, MfeI, MflI, MlsI, MluI, MluNI, Mly113I, MmeI, MnlI, Mph1103I, MroI, MroNI, MroXI, MscI, MseI, MslI, Msp171, MspA1I, MspCI, MspI, MspR9I, MstI, MunI, Mva1269I, MvaI, MvnI, MwoI, NaeI, NarI, NciI, Ncol, NdeI, NdeII, NgoAIV, NgoMIV (previously known as NgoMI), NheI, NlaIII, NlaIV, NotI, NruGI, NruI, NsbI, NsiI, NspBII, NspI, NspV, PacI, PaeI, PaeR7I, PagI, PalI, PauI, Pfl1108I, Pfl23II, PflFI, PflMI, PinAI, Ple19I, PleI, PmaCI, Pme55I, PmeI, PmlI, Ppu10I, PpuMI, PshAI, PshBI, Psp124BI, Psp1406I, Psp5II, PspAI, PspEI, PspLI, PspN4I, PspOMI, PspPPI, PstI, PvuI, PvuII, RcaI, RleAI, RsaI, RsrII, SacI, SacII, SalI, SanDI, SapI, Sau3AI, Sau96I, SauI, SbfI, ScaI, SchI, ScrFI, SdaI, SduI, SecI, SexAI, SfaNI, SfcI, SfeI, SfiI, SfoI, Sfr274I, Sfr303I, SfuI, SgfI, SgrAI, SimI, SinI, SmaI, SmiI, SmlII, SnaBI, SnaI, SpeI, SphI, SplI, SrfI, Sse8387I, Sse8647I, Sse9I, SseBI, SspBI, SspI, SstI, SstII, StuI, StyI, SunI, SwaI, TaiI, TaqI, TaqI, TatI, TauI, TfiI, ThaI, TruII, Tru9I, TscI, TseI, Tsp45I, Tsp4CI, Tsp509I, TspEI, TspRI, Tth111I, Tth111II, TthHB8I, UbaDI, UbaEI, UbaLI, UbaOI, Van91I, Vha4641, VneI, VspI; XagI, XbaI, XcmI, XhoI, XhoII, XmaCI, XmaI, XmaIII XmnI, Zsp2I, Tn5 transposases, DNase, or micrococcal nuclease (MNase).

12. A method for identifying one or more specific genomic target regions and molecules interacting therewith comprising:
contacting a recombinant nuclease-deficient Cas9 fusion protein (dCas9 fusion protein) modified to comprise a biotinylation sequence and one or more sequence-specific guide RNAs to the one or more specific genomic DNA targets in cells to form a CRISPR complex;
in vivo biotinylating the dCas9 fusion protein with a biotin ligase;
fragmenting the genomic DNA around the CRISPR complex;
isolating the CRISPR complex with a streptavidin or an avidin; and
determining an identity of one or more proteins, DNAs, or RNAs in the CRISPR complex.

13. The method of claim 12, wherein fragmenting the genomic DNA in the cells under conditions in which the genomic DNA and molecules interacting therewith are maintained in the CRISPR complex.

14. The method of claim 12, wherein the one or more sequence-specific guide RNAs are programmable sequence-specific guide RNAs (sgRNAs).

15. The method of claim 12, wherein the dCas9 fusion protein is biotinylated and further comprises an isolatable peptide tag at the N-,C-terminus or other regions of the dCas9 protein selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both; and optionally the dCas9 fusion protein is bound with the streptavidin or avidin that has been conjugated to a detectable label selected from at least one of an electrochemiluminescence label, an enzyme label, a fluorophore, a latex particle, a magnetic particle, a radioactive element, a phosphorescent dye, a dye, a gold, silver, or selenium particle, or a ruthenium or osmium metal chelate; and optionally the streptavidin or avidin is bound to a solid support, a chip, a substrate, a column, a well, or beads.

16. The method of claim 12, further comprising performing a chemical treatment that maintains the interaction of genomic DNA and molecules interacting therewith in the CRISPR complex.

17. The method of claim 12, wherein the recombinant nuclease-deficient Cas9 fusion protein is SEQ ID NO:334.

18. The method of claim 12, further comprising expressing in the cells a biotin ligase capable of biotinylating the recombinant nuclease-deficient Cas9 fusion protein.

19. The method of claim 12, further comprising at least one of: (1) capturing in situ one or more locus-specific chromatin interactions by biotinylated dCas9 fusion protein; (2) using biotinylated dCas9-mediated capture of the binding cluster at or around the sequence-specific guide RNA; (3) identifying cis-regulatory elements (CRE)-associated protein complexes to identify proteins or nucleic acids of the CRISPR complex; (4) using the CRISPR complex for CRISPR affinity purification in situ of regulatory elements (CAPTURE)-proteomics to identify known and new regulators of at least one of genes, promoters, or enhancers by: cross-linking the CRISPR complex, fragmenting the complex, dCas9 fusion protein affinity purification, and sequencing the nucleic acids isolated therewith, western blot, or peptide digestion with multiplex identification by proteomic profiling; (5) using CAPTURE-3C-seq to identify locus-specific long-range DNA interactions by crosslinking of the CRISPR complex, enzymatic digestion of nucleic acids, proximity ligation of the nucleic acids, fragmentation of the genomic DNA, dCas9 fusion protein affinity purification, and pair-end sequencing to identify tethered long-range interactions; (6) using biotinylated dCas9-mediated in situ capture of a disease-associated cis-regulatory elements (CRE) to measure cis-transcription factors, RNA complexes, and long-range DNA interactions that contribute to the disease phenotypes; (7) detecting the CRISPR complex in situ; (8) using multiplexed CAPTURE of developmentally regulated super-enhancers during differentiation; (9) identifying nucleic acids, peptides, proteins, by at least one of mass spectrometry (MS)-based proteomics, MS-MS, MALDI, MALDI-TOF, multiplex proteomic identification, immunoblot, ELISA, nucleotide sequence analysis, microarray analysis, or PCR; or (10) using multiplexed CAPTURE using 2, 3, 4, 5, 6, 7, 8, 9, 10, 15, 20, 25, 30, 35, 40, 45, 50, 60, 70, 75, 80, 90, 100, 200, 300, 400, 500, 600, 700, 800, 900, 1,000, 2,000, 3,000, 4000, 5,000, 6,000, 7,000, 8,000, 9,000, 10,000, or more sgRNAs in a pool to target multiple genomic regions, including multiple cis-elements at the same enhancer cluster or multiple independent enhancers.

20. The method of claim 12, further comprising significantly enriching molecular interactions at one or more genomic targets by comparing the molecules in the CRISPR complex when compared to one or more negative controls.

21. The method of claim 12, wherein the negative controls include one or more of the following: cells expressing biotin ligase (BirA) only, cells expression BirA and dCas9 fusion protein, cells expression BirA, dCas9 and the non-targeting sgRNA (sgGal4), and cells expression BirA, dCas9, one or more sequence-specific sgRNAs, and knockout of the sgRNA targeting sequences in the genome.

22. A method for identifying one or more long-range DNA interactions (or looping) with a CRISPR complex comprising:
contacting a recombinant nuclease-deficient Cas9 fusion protein (dCas9 fusion protein) modified to comprise a biotinylation sequence or another isolatable tag and one or more sequence-specific guide RNAs to the one or more specific genomic DNA targets in cells to form a CRISPR complex;
in vivo biotinylating the dCas9 fusion protein with a biotin ligase;
enzymatically digesting genomic DNA with a restriction enzyme or other nucleases;
proximity ligating one or more nucleic acids in the CRISPR complex;
isolating the CRISPR complex by affinity purification with a streptavidin or an avidin; and
pair-end sequencing to identify tethered long-range interactions in the CRISPR complex.

23. The method of claim 22, wherein the restriction enzyme is selected from at least one of: AatI, AatII, AauI, Acc113I, Acc16I, Acc65I, AccB11, AccB7I, AccBSI, AccI, AccII, AccIII, AceIII, AciI, AclI, AclNI, AclWI, AcsI, AcyI, AdeI, AfaI, AfeI, AflII, AflIII, AgeI, AhaIII, AhdI, AluI, Alw21I, Alw26I, Alw44I, AlwI, AlwNI, Ama87I, AocI, Aor51HI, ApaBI, Apal, ApaLI, ApoI, AscI, Asel, AsiAI, AsnI, Asp700I, Asp7181, AspEI, AspHI, AspI, AspLEI, AspS9I, AsuC2I, AsuHPI, AsuI, AsuII, AsuNHI, AvaI, Avail, AvaIII, AviII, AvrII, AxyI, BaeI, BalI, BamHI, BanI, BanII, BanIII, BbeI, BbiII, BbrPI, BbsI, BbuI, Bbv12I, BbvCI, BbvI, BbvII, BccI, Bce83I, BcefI, BcgI, BciVI, BclI, BcnI, BcoI, BcuI, BetI, BfaI, BfiI, BfmI, BfrI, BglI, BglII, BinI, BlnI, BlpI, Bme18I, BmgI, BmrI, BmyI, BpiI, BplI, BpmI, Bpu10I, Bpu1102I, Bpu14I, BpuAI, Bsa29I, BsaAI, BsaBI, BsaHI, BsaI, BsaJI, BsaMI, BsaOI, BsaWI, BsaXI, BsbI, Bsc4I, BscBI, BscCI, BscFI, BscGI, BscI, Bse181, Bse1I, Bse21I, Bse3DI, Bse8I, BseAI, BseCI, BseDI, BseGI, BseLI, BseMII, BseNI, BsePI, BseRI, BseX3I, BsgI, Bsh1236I, Bsh1285I, Bsh1365I, BshI, BshNI, BsiBI, BsiCI, BsiEI, BsiHKAI, BsiI, BsiLI, BsiMI, BsiQI, BsiSI, BsiWI, BsiXI, BsiYI, BsiZI, BslI, BsmAI, BsmBI, BsmFI, BsmI, BsoBI, Bsp106I, Bsp1191, Bsp120I, Bsp1286I, Bsp13I, Bsp1407I, Bsp143I, Bsp143II, Bsp1720I, Bsp19I, Bsp24I, Bsp68I, BspA2I, BspCI, BspDI, BspEI, BspGI, BspHI, BspLI, BspLU11I, BspMI, BspMII, BspTI, BspXI, BsrBI, BsrBRI, BsrDI, BsrFI, BsrGI, BsrI, BsrSI, BssAI, BssHII, BssKI, BssNAI, BssSI, BssT1I, Bst1107I, Bst2BI, Bst2UI, Bst4CI, Bst71I, Bst98I, BstACI, BstAPI, BstBAI, BstBI, BstDEI, BstDSI, BstEII, BstF5I, BstH2I, BstHPI, BstMCI, BstNI, BstNSI, BstOI, BstPI, BstSFI, BstSNI, BstUI, BstX2I, BstXI, BstYI, BstZ17, BstZI, Bsu15I, Bsu36I, Bsu6I, BsuRI, BtgI, BtsI, Cac8I, CauII, CbiI, CciNI, CelII, CfoI, Cfr10I, Cfr13I, Cfr42I, Cfr9I, CfrI, CjeI, CjePI, ClaI, CpoI, Csp45I, Csp6I, CspI, CviJI, CviRI, CvnI, DdeI, DpnI, DpnII, DraI, DraII, DraIII, DrdI, DrdII, DsaI, DseDI, EaeI, EagI, Eam1104I, Eaml 11051, EarI, EciI, Ec136II, EclHKI, EclXI, Eco105I, Eco130I, Eco147I, Eco24I, Eco255I, Eco31I, Eco32I, Eco47I, Eco47III, Eco52I, Eco57I, Eco64I, Eco72I, Eco81I, Eco88I, Eco91I, EcoICRI, EcoNI, EcoO109I, EcoO65I, EcoRI, EcoRII, EcoRV, EcoT14I, EcoT22I, EcoT38I, EgeI, EheI, ErhI, Esp1396I, Esp3I, EspI, FauI, FauNDI, FbaI, FinI, Fnu4HI, FnuDUII, FokI, FriOI, FseI, Fsp4HI, FspI, GdiII, GsuI, HaeI, HaeII, HaeIII, HaeIV, HapII, HgaI, HgiAI, HgiCI, HgiEI, HgiEII, HgiJII, HhaI, Hin1I, Hin2I, Hin4I, Hin6I, HincII, HindII, HindIII, HinfI, HinP1I, HpaI, HpaII, HphI, Hsp92I, Hsp92II, HspAI, ItaI, KasI, Kpn2I, KpnI, Ksp22I, Ksp632I, KspAI, KspI, Kzo9I, LspI, MaeI, MaeII, MaeIII, MamI, MbiI, MboI, MboII, McrI, MfeI, MflI, MlsI, MluI, MluNI, Mly113I, MmeI, MnlI, Mph1103I, MroI, MroNI, MroXI, MscI, MseI, MslI, Msp171, MspA1I, MspCI, MspI, MspR9I, MstI, MunI, Mva1269I, MvaI, MvnI, MwoI, NaeI, NarI, NciI, Ncol, NdeI, NdeII, NgoAIV, NgoMIV (previously known as NgoMI), NheI, NlaIII, NlaIV, NotI, NruGI, NruI, NsbI, NsiI, NspBII, NspI, NspV, PacI, PaeI, PaeR7I, PagI, PalI, PauI, Pfl1108I, Pfl23II, PflFI, PflMI, PinAI, Ple19I, PleI, PmaCI, Pme55I, PmeI, PmlI, Ppu10I, PpuMI, PshAI, PshBI, Psp124BI, Psp1406I, Psp5II, PspAI, PspEI, PspLI, PspN4I, PspOMI, PspPPI, PstI, PvuI, PvuII, RcaI, RleAI, RsaI, RsrII, SacI, SacII, SalI, SanDI, SapI, Sau3AI, Sau96I, SauI, SbfI, ScaI, SchI, ScrFI, SdaI, SduI, SecI, SexAI, SfaNI, SfcI, SfeI, SfiI, SfoI, Sfr274I, Sfr303I, SfuI, SgfI, SgrAI, SimI, SinI, SmaI, SmiI, SmlI, SnaBI, SnaI, SpeI, SphI, SplI, SrfI, Sse8387I, Sse8647I, Sse9I, SseBI, SspBI, SspI, SstI, SstII, StuI, StyI, SunI, SwaI, TaiI, TaqI, TaqII, TatI, TauI, TfiI, ThaI, TruII, Tru9I, TscI, TseI, Tsp45I, Tsp4CI, Tsp509I, TspEI, TspRI, Tth111I, Tth111II, TthHB8I, UbaDI, UbaEI, UbaLI, UbaOI, Van91I, Vha4641, VneI, VspI; XagI, XbaI, XcmI, XhoI, XhoII, XmaCI, XmaI, XmaIII XmnI, Zsp2I, Tn5 transposases, DNase, or micrococcal nuclease (MNase).

24. The method of claim 22, further comprising the step of crosslinking the CRISPR complex.

25. The method of claim 22, further comprising fragmenting the genomic DNA after isolating the CRISPR complex.

26. The method of claim 22, wherein the step of affinity purification of the CRISPR complex is performed using a isolatable tag selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both.

27. A nucleic acid vector encoding a recombinant nuclease-deficient Cas9 fusion protein (dCas9) modified to comprise a biotinylation sequence and a tag sequence.

28. The nucleic acid vector of claim 27, further comprising a biotin ligase gene.

29. The nucleic acid vector of claim 27, wherein the tag sequence is selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in prokaryotic cells, eukaryotic cells, or both.

30. The nucleic acid vector of claim 27, wherein the nucleic acid has SEQ ID NO:333.

31. A protein comprising a recombinant nuclease-deficient Cas9 fusion protein (dCas9 fusion protein) modified to comprise a biotinylation sequence and a tag sequence.

32. The protein of claim 31, wherein the tag sequence is at the N- or C-terminus, or in other regions of the dCas9 protein.

33. The protein of claim 31, wherein the tag sequence is selected from at least one of FLAG tag, a myc, a His-tag, Strep tag, a BioTAP tag, a calmodulin-binding peptide tag, a GST tag, an Maltose Binding Protein tag, a Halo tag, a Hemagglutinin A tag, or a biotinylation targeting sequence that is recognized by endogenous biotin ligases in both prokaryotic and eukaryotic cells.

34. The protein of claim 31, wherein the dCas9 fusion protein is bound to a solid support, a chip, a substrate, a column, a well, or beads by streptavidin or avidin.

35. The protein of claim 31, wherein the protein has amino acid sequence SEQ ID NO:334.


SEQUENCE LISTING (334 sequences)

SEQ ID NO: 1 - DNA, 63 bp, Artificial Sequence
  Synthetic primer
ggagaaccaccttgttggctaaactccacccatgggtgtttaagagctatgctggaaacagca

SEQ ID NO: 2 - DNA, 37 bp, Artificial Sequence
  Synthetic primer
ctagtactcgagaaaaaaagcaccgactcggtgccac

SEQ ID NO: 3 - DNA, 63 bp, Artificial Sequence
  Synthetic primer
ggagaaccaccttgttggctcctagtccagacgccatgtttaagagctatgctggaaacagca

SEQ ID NO: 4 - DNA, 37 bp, Artificial Sequence; identical to SEQ ID NO: 2
  Synthetic primer

SEQ ID NO: 5 - DNA, 64 bp, Artificial Sequence
  Synthetic primer
ggagaaccaccttgttggccctgtggagccacaccctagtttaagagctatgctggaaacagca

SEQ ID NO: 6 - DNA, 37 bp, Artificial Sequence; identical to SEQ ID NO: 2
  Synthetic primer

SEQ ID NO: 7 - DNA, 63 bp, Artificial Sequence
  Synthetic primer
ggagaaccaccttgttggtctgccgttactgccctgtgtttaagagctatgctggaaacagca

SEQ ID NO: 8 - DNA, 37 bp, Artificial Sequence; identical to SEQ ID NO: 2
  Synthetic primer

SEQ ID NO: 9 - DNA, 64 bp, Artificial Sequence
  Synthetic primer
ggagaaccaccttgttggcaataggtatatgaggagacgtttaagagctatgctggaaacagca

SEQ ID NO: 10 - DNA, 37 bp, Artificial Sequence; identical to SEQ ID NO: 2
  Synthetic primer

SEQ ID NO: 11 - DNA, 64 bp, Artificial Sequence
  Synthetic primer
ggagaaccaccttgttggttgtgtagaaaccaagcgtggtttaagagctatgctggaaacagca

SEQ ID NO: 12 - DNA, 37 bp, Artificial Sequence; identical to SEQ ID NO: 2
  Synthetic primer

SEQ ID NO: 13 - DNA, 64 bp, Artificial Sequence
  Synthetic primer
ggagaaccaccttgttggagtccaagcatgagcagttcgtttaagagctatgctggaaacagca

SEQ ID NO: 14 - DNA, 37 bp, Artificial Sequence; identical to SEQ ID NO: 2
  Synthetic primer

SEQ ID NO: 15 - DNA, 63 bp, Artificial Sequence
  Synthetic primer
ggagaaccaccttgttggtggcctctatacctagaaggtttaagagctatgctggaaacagca

SEQ ID NO: 16 - DNA, 37 bp, Artificial Sequence; identical to SEQ ID NO: 2
  Synthetic primer

SEQ ID NO: 17 - DNA, 64 bp, Artificial Sequence
  Synthetic primer
ggagaaccaccttgttggtataatgtgctctgtcccccgtttaagagctatgctggaaacagca

SEQ ID NO: 18 - DNA, 37 bp, Artificial Sequence; identical to SEQ ID NO: 2
  Synthetic primer

SEQ ID NO: 19 - DNA, 64 bp, Artificial Sequence
  Synthetic primer
ggagaaccaccttgttggaatagtgtttagcatccagcgtttaagagctatgctggaaacagca

SEQ ID NO: 20 - DNA, 37 bp, Artificial Sequence; identical to SEQ ID NO: 2
  Synthetic primer

SEQ ID NO: 21 - DNA, 63 bp, Artificial Sequence
  Synthetic primer
ggagaaccaccttgttgctttatgatgccgtttgagggtttaagagctatgctggaaacagca

SEQ ID NO: 22 - DNA, 37 bp, Artificial Sequence; identical to SEQ ID NO: 2
  Synthetic primer

SEQ ID NO: 23 - DNA, 63 bp, Artificial Sequence
  Synthetic primer
ggagaaccaccttgttggagagatagaccatgagtaggtttaagagctatgctggaaacagca

SEQ ID NO: 24 - DNA, 37 bp, Artificial Sequence; identical to SEQ ID NO: 2
  Synthetic primer

SEQ ID NO: 25 - DNA, 64 bp, Artificial Sequence
  Synthetic primer
ggagaaccaccttgttggaggaatcattctgtggataagtttaagagctatgctggaaacagca

SEQ ID NO: 26 - DNA, 37 bp, Artificial Sequence; identical to SEQ ID NO: 2
  Synthetic primer

SEQ ID NO: 27 - DNA, 63 bp, Artificial Sequence
  Synthetic primer
ggagaaccaccttgttggaagtctatgactgtaaattgtttaagagctatgctggaaacagca

SEQ ID NO: 28 - DNA, 37 bp, Artificial Sequence; identical to SEQ ID NO: 2
  Synthetic primer

SEQ ID NO: 29 - DNA, 63 bp, Artificial Sequence
  Synthetic primer
ggagaaccaccttgttggcccctagctgggggtataggtttaagagctatgctggaaacagca

SEQ ID NO: 30 - DNA, 37 bp, Artificial Sequence; identical to SEQ ID NO: 2
  Synthetic primer

SEQ ID NO: 31 - DNA, 64 bp, Artificial Sequence
  Synthetic primer
ggagaaccaccttgttggcccactcagcagctatgagagtttaagagctatgctggaaacagca

SEQ ID NO: 32 - DNA, 37 bp, Artificial Sequence; identical to SEQ ID NO: 2
  Synthetic primer

SEQ ID NO: 33 - DNA, 64 bp, Artificial Sequence
  Synthetic primer
ggagaaccaccttgttggtctccctcccattcccgagcgtttaagagctatgctggaaacagca

SEQ ID NO: 34 - DNA, 37 bp, Artificial Sequence; identical to SEQ ID NO: 2
  Synthetic primer

SEQ ID NO: 35 - DNA, 64 bp, Artificial Sequence
  Synthetic primer
ggagaaccaccttgttggtgcccccaccttacagggacgtttaagagctatgctggaaacagca

SEQ ID NO: 36 - DNA, 37 bp, Artificial Sequence; identical to SEQ ID NO: 2
  Synthetic primer

SEQ ID NO: 37 - DNA, 63 bp, Artificial Sequence
  Synthetic primer
ggagaaccaccttgttgggagcccttttgattgaagggtttaagagctatgctggaaacagca

SEQ ID NO: 38 - DNA, 37 bp, Artificial Sequence; identical to SEQ ID NO: 2
  Synthetic primer

SEQ ID NO: 39 - DNA, 63 bp, Artificial Sequence
  Synthetic primer
ggagaaccaccttgttggctttagtgtaagcgaggtcgtttaagagctatgctggaaacagca

SEQ ID NO: 40 - DNA, 37 bp, Artificial Sequence; identical to SEQ ID NO: 2
  Synthetic primer

SEQ ID NO: 41 - DNA, 63 bp, Artificial Sequence
  Synthetic primer
ggagaaccaccttgttggtacaatagtataaccccttgtttaagagctatgctggaaacagca

SEQ ID NO: 42 - DNA, 37 bp, Artificial Sequence; identical to SEQ ID NO: 2
  Synthetic primer

SEQ ID NO: 43 - DNA, 63 bp, Artificial Sequence
  Synthetic primer
ggagaaccaccttgttggctgggcttctgttgcagtagtttaagagctatgctggaaacagca

SEQ ID NO: 44 - DNA, 37 bp, Artificial Sequence; identical to SEQ ID NO: 2
  Synthetic primer

SEQ ID NO: 45 - DNA, 64 bp, Artificial Sequence
  Synthetic primer
ggagaaccaccttgttggagatcaaataacagtcctcagtttaagagctatgctggaaacagca

SEQ ID NO: 46 - DNA, 37 bp, Artificial Sequence; identical to SEQ ID NO: 2
  Synthetic primer

SEQ ID NO: 47 - DNA, 65 bp, Artificial Sequence
  Synthetic primer
ggagaaccaccttgttggttagggttagggttagggttagtttaagagctatgctggaaacagca

SEQ ID NO: 48 - DNA, 37 bp, Artificial Sequence; identical to SEQ ID NO: 2
  Synthetic primer

SEQ ID NO: 49 - DNA, 64 bp, Artificial Sequence
  Synthetic primer
ggagaaccaccttgttggaacgactagttaggcgtgtagtttaagagctatgctggaaacagca

SEQ ID NO: 50 - DNA, 37 bp, Artificial Sequence; identical to SEQ ID NO: 2
  Synthetic primer

SEQ ID NO: 51 - DNA, 63 bp, Artificial Sequence
  Synthetic primer
ggagaaccaccttgttggtttctctatgaagtgaagcgtttaagagctatgctggaaacagca

SEQ ID NO: 52 - DNA, 37 bp, Artificial Sequence; identical to SEQ ID NO: 2
  Synthetic primer

SEQ ID NO: 53 - DNA, 64 bp, Artificial Sequence
  Synthetic primer
ggagaaccaccttgttggctctctaccctcggggcgatgtttaagagctatgctggaaacagca

SEQ ID NO: 54 - DNA, 37 bp, Artificial Sequence; identical to SEQ ID NO: 2
  Synthetic primer

SEQ ID NO: 55 - DNA, 64 bp, Artificial Sequence
  Synthetic primer
ggagaaccaccttgttggctcaaactatgcccacctgcgtttaagagctatgctggaaacagca

SEQ ID NO: 56 - DNA, 37 bp, Artificial Sequence; identical to SEQ ID NO: 2
  Synthetic primer

SEQ ID NO: 57 - DNA, 63 bp, Artificial Sequence
  Synthetic primer
ggagaaccaccttgttgggacttgaaagatgcagggggtttaagagctatgctggaaacagca

SEQ ID NO: 58 - DNA, 37 bp, Artificial Sequence; identical to SEQ ID NO: 2
  Synthetic primer

SEQ ID NO: 59 - DNA, 64 bp, Artificial Sequence
  Synthetic primer
ggagaaccaccttgttggtactaattaacttatagttggtttaagagctatgctggaaacagca

SEQ ID NO: 60 - DNA, 37 bp, Artificial Sequence; identical to SEQ ID NO: 2
  Synthetic primer

SEQ ID NO: 61 - DNA, 64 bp, Artificial Sequence
  Synthetic primer
ggagaaccaccttgttggcaaaggatgaatgtgtcgacgtttaagagctatgctggaaacagca

SEQ ID NO: 62 - DNA, 37 bp, Artificial Sequence; identical to SEQ ID NO: 2
  Synthetic primer

SEQ ID NO: 63 - DNA, 64 bp, Artificial Sequence
  Synthetic primer
ggagaaccaccttgttggcacaaggctataatgaacgcgtttaagagctatgctggaaacagca

SEQ ID NO: 64 - DNA, 37 bp, Artificial Sequence; identical to SEQ ID NO: 2
  Synthetic primer

SEQ ID NO: 65 - DNA, 63 bp, Artificial Sequence
  Synthetic primer
ggagaaccaccttgttggagagttttcctagcgcagagtttaagagctatgctggaaacagca

SEQ ID NO: 66 - DNA, 37 bp, Artificial Sequence; identical to SEQ ID NO: 2
  Synthetic primer

SEQ ID NO: 67 - DNA, 64 bp, Artificial Sequence
  Synthetic primer
ggagaaccaccttgttggtaagagtcgagtattggcgagtttaagagctatgctggaaacagca

SEQ ID NO: 68 - DNA, 37 bp, Artificial Sequence; identical to SEQ ID NO: 2
  Synthetic primer

SEQ ID NO: 69 - DNA, 63 bp, Artificial Sequence
  Synthetic primer
ggagaaccaccttgttggagcggcggcgaaccctcgggtttaagagctatgctggaaacagca

SEQ ID NO: 70 - DNA, 37 bp, Artificial Sequence; identical to SEQ ID NO: 2
  Synthetic primer

SEQ ID NO: 71 - DNA, 64 bp, Artificial Sequence
  Synthetic primer
ggagaaccaccttgttggctgggctttgctaagtccgtgtttaagagctatgctggaaacagca

SEQ ID NO: 72 - DNA, 37 bp, Artificial Sequence; identical to SEQ ID NO: 2
  Synthetic primer

SEQ ID NO: 73 - DNA, 64 bp, Artificial Sequence
  Synthetic primer
ggagaaccaccttgttggtctctcacagaagggatcgcgtttaagagctatgctggaaacagca

SEQ ID NO: 74 - DNA, 37 bp, Artificial Sequence; identical to SEQ ID NO: 2
  Synthetic primer

SEQ ID NO: 75 - DNA, 64 bp, Artificial Sequence
  Synthetic primer
ggagaaccaccttgttggtttcccctagacaatgacgggtttaagagctatgctggaaacagca

SEQ ID NO: 76 - DNA, 37 bp, Artificial Sequence; identical to SEQ ID NO: 2
  Synthetic primer

SEQ ID NO: 77 - DNA, 64 bp, Artificial Sequence
  Synthetic primer
ggagaaccaccttgttggacctgcctcagtcttcaaacgtttaagagctatgctggaaacagca

SEQ ID NO: 78 - DNA, 37 bp, Artificial Sequence; identical to SEQ ID NO: 2
  Synthetic primer

SEQ ID NO: 79 - DNA, 64 bp, Artificial Sequence
  Synthetic primer
ggagaaccaccttgttggagacactgaattgactgtgtgtttaagagctatgctggaaacagca

SEQ ID NO: 80 - DNA, 37 bp, Artificial Sequence; identical to SEQ ID NO: 2
  Synthetic primer

SEQ ID NO: 81 - DNA, 63 bp, Artificial Sequence
  Synthetic primer
ggagaaccaccttgttgggtctacagaatgagttctagtttaagagctatgctggaaacagca

SEQ ID NO: 82 - DNA, 37 bp, Artificial Sequence; identical to SEQ ID NO: 2
  Synthetic primer

SEQ ID NO: 83 - DNA, 63 bp, Artificial Sequence
  Synthetic primer
ggagaaccaccttgttggaaggcatagagctttgtacgtttaagagctatgctggaaacagca

SEQ ID NO: 84 - DNA, 37 bp, Artificial Sequence; identical to SEQ ID NO: 2
  Synthetic primer

SEQ ID NO: 85 - DNA, 64 bp, Artificial Sequence
  Synthetic primer
ggagaaccaccttgttggacaagggtcgctcgccctgcgtttaagagctatgctggaaacagca

SEQ ID NO: 86 - DNA, 37 bp, Artificial Sequence; identical to SEQ ID NO: 2
  Synthetic primer

SEQ ID NO: 87 - DNA, 63 bp, Artificial Sequence
  Synthetic primer
ggagaaccaccttgttggtttagtccaccgctagctagtttaagagctatgctggaaacagca

SEQ ID NO: 88 - DNA, 37 bp, Artificial Sequence; identical to SEQ ID NO: 2
  Synthetic primer

SEQ ID NO: 89 - DNA, 64 bp, Artificial Sequence
  Synthetic primer
ggagaaccaccttgttggtagcactagaacctaacctcgtttaagagctatgctggaaacagca

SEQ ID NO: 90 - DNA, 37 bp, Artificial Sequence; identical to SEQ ID NO: 2
  Synthetic primer

SEQ ID NO: 91 - DNA, 64 bp, Artificial Sequence
  Synthetic primer
ggagaaccaccttgttggactcacagtaagaaagctgtgtttaagagctatgctggaaacagca

SEQ ID NO: 92 - DNA, 37 bp, Artificial Sequence; identical to SEQ ID NO: 2
  Synthetic primer

SEQ ID NO: 93 - DNA, 64 bp, Artificial Sequence
  Synthetic primer
ggagaaccaccttgttggatattgggtggtttacagctgtttaagagctatgctggaaacagca

SEQ ID NO: 94 - DNA, 37 bp, Artificial Sequence; identical to SEQ ID NO: 2
  Synthetic primer

SEQ ID NO: 95 - DNA, 63 bp, Artificial Sequence
  Synthetic primer
ggagaaccaccttgttggtgggcttctctgctgtcttgtttaagagctatgctggaaacagca

SEQ ID NO: 96 - DNA, 37 bp, Artificial Sequence; identical to SEQ ID NO: 2
  Synthetic primer

SEQ ID NO: 97 - DNA, 64 bp, Artificial Sequence
  Synthetic primer
ggagaaccaccttgttggcaggctcacagctcgggaccgtttaagagctatgctggaaacagca

SEQ ID NO: 98 - DNA, 37 bp, Artificial Sequence; identical to SEQ ID NO: 2
  Synthetic primer

SEQ ID NO: 99 - DNA, 63 bp, Artificial Sequence
  Synthetic primer
ggagaaccaccttgttggagtgctgtctaggccttaggtttaagagctatgctggaaacagca

SEQ ID NO: 100 - DNA, 37 bp, Artificial Sequence; identical to SEQ ID NO: 2
  Synthetic primer

SEQ ID NO: 101 - DNA, 63 bp, Artificial Sequence
  Synthetic primer
ggagaaccaccttgttggaacagtgccataggttagtgtttaagagctatgctggaaacagca

SEQ ID NO: 102 - DNA, 37 bp, Artificial Sequence; identical to SEQ ID NO: 2
  Synthetic primer

SEQ ID NO: 103 - DNA, 64 bp, Artificial Sequence
  Synthetic primer
ggagaaccaccttgttggaaaccactctagggaagttcgtttaagagctatgctggaaacagca

SEQ ID NO: 104 - DNA, 37 bp, Artificial Sequence; identical to SEQ ID NO: 2
  Synthetic primer

SEQ ID NO: 105 - DNA, 63 bp, Artificial Sequence
  Synthetic primer
ggagaaccaccttgttggggtggagaaacccaacggggtttaagagctatgctggaaacagca

SEQ ID NO: 106 - DNA, 37 bp, Artificial Sequence; identical to SEQ ID NO: 2
  Synthetic primer

SEQ ID NO: 107 - DNA, 64 bp, Artificial Sequence
  Synthetic primer
ggagaaccaccttgttggcccccaccaggtgggggtgagtttaagagctatgctggaaacagca

SEQ ID NO: 108 - DNA, 37 bp, Artificial Sequence; identical to SEQ ID NO: 2
  Synthetic primer

SEQ ID NO: 109 - DNA, 64 bp, Artificial Sequence
  Synthetic primer
ggagaaccaccttgttggcagtgtaccttgtatccatagtttaagagctatgctggaaacagca

SEQ ID NO: 110 - DNA, 37 bp, Artificial Sequence; identical to SEQ ID NO: 2
  Synthetic primer

SEQ ID NO: 111 - DNA, 64 bp, Artificial Sequence
  Synthetic primer
ggagaaccaccttgttggtcctcggaatggttggcgaggtttaagagctatgctggaaacagca

SEQ ID NO: 112 - DNA, 37 bp, Artificial Sequence; identical to SEQ ID NO: 2
  Synthetic primer

SEQ ID NO: 113 - DNA, 64 bp, Artificial Sequence
  Synthetic primer
ggagaaccaccttgttggtgcttggcagttaaggcttcgtttaagagctatgctggaaacagca

SEQ ID NO: 114 - DNA, 37 bp, Artificial Sequence; identical to SEQ ID NO: 2
  Synthetic primer

SEQ ID NO: 115 - DNA, 64 bp, Artificial Sequence
  Synthetic primer
ggagaaccaccttgttggttaggggactatgatggtgtgtttaagagctatgctggaaacagca

SEQ ID NO: 116 - DNA, 37 bp, Artificial Sequence; identical to SEQ ID NO: 2
  Synthetic primer

SEQ ID NO: 117 - DNA, 63 bp, Artificial Sequence
  Synthetic primer
ggagaaccaccttgttggtaaaagcaagtccaccagcgtttaagagctatgctggaaacagca

SEQ ID NO: 118 - DNA, 37 bp, Artificial Sequence; identical to SEQ ID NO: 2
  Synthetic primer

SEQ ID NO: 119 - DNA, 64 bp, Artificial Sequence
  Synthetic primer
ggagaaccaccttgttggcaatttttctgggtctaaaggtttaagagctatgctggaaacagca

SEQ ID NO: 120 - DNA, 37 bp, Artificial Sequence; identical to SEQ ID NO: 2
  Synthetic primer

SEQ ID NO: 121 - DNA, 64 bp, Artificial Sequence
  Synthetic primer
ggagaaccaccttgttggaatgcacttgggtacaaaaggtttaagagctatgctggaaacagca

SEQ ID NO: 122 - DNA, 37 bp, Artificial Sequence; identical to SEQ ID NO: 2
  Synthetic primer

SEQ ID NO: 123 - DNA, 64 bp, Artificial Sequence
  Synthetic primer
ggagaaccaccttgttggcggacgtggggctgtggctcgtttaagagctatgctggaaacagca

SEQ ID NO: 124 - DNA, 37 bp, Artificial Sequence; identical to SEQ ID NO: 2
  Synthetic primer

SEQ ID NO: 125 - DNA, 64 bp, Artificial Sequence
  Synthetic primer
ggagaaccaccttgttggaactggcggcggccggtactgtttaagagctatgctggaaacagca

SEQ ID NO: 126 - DNA, 37 bp, Artificial Sequence; identical to SEQ ID NO: 2
  Synthetic primer

SEQ ID NO: 127 - DNA, 64 bp, Artificial Sequence
  Synthetic primer
ggagaaccaccttgttggtcgtttttagggtaaggtacgtttaagagctatgctggaaacagca

SEQ ID NO: 128 - DNA, 37 bp, Artificial Sequence; identical to SEQ ID NO: 2
  Synthetic primer

SEQ ID NO: 129 - DNA, 63 bp, Artificial Sequence
  Synthetic primer
ggagaaccaccttgttggactcagcctctcaacttaagtttaagagctatgctggaaacagca

SEQ ID NO: 130 - DNA, 37 bp, Artificial Sequence; identical to SEQ ID NO: 2
  Synthetic primer

SEQ ID NO: 131 - DNA, 64 bp, Artificial Sequence
  Synthetic primer
ggagaaccaccttgttggtagttgctgaaatagggaaggtttaagagctatgctggaaacagca

SEQ ID NO: 132 - DNA, 37 bp, Artificial Sequence; identical to SEQ ID NO: 2
  Synthetic primer

SEQ ID NO: 133 - DNA, 25 bp, Artificial Sequence
  Synthetic primer
caccgtttgggacatgcggatgcac

SEQ ID NO: 134 - DNA, 25 bp, Artificial Sequence
  Synthetic primer
aaacgtgcatccgcatgtcccaaac

SEQ ID NO: 135 - DNA, 25 bp, Artificial Sequence
  Synthetic primer
caccgcctccatctggtccattatc

SEQ ID NO: 136 - DNA, 25 bp, Artificial Sequence
  Synthetic primer
aaacgataatggaccagatggaggc

SEQ ID NO: 137 - DNA, 25 bp, Artificial Sequence
  Synthetic primer
caccggtgttccattggtcttagag

SEQ ID NO: 138 - DNA, 25 bp, Artificial Sequence
  Synthetic primer
aaacctctaagaccaatggaacacc

SEQ ID NO: 139 - DNA, 25 bp, Artificial Sequence
  Synthetic primer
caccggtcactctcaagtgttccat

SEQ ID NO: 140 - DNA, 25 bp, Artificial Sequence
  Synthetic primer
aaacatggaacacttgagagtgacc

SEQ ID NO: 141 - DNA, 25 bp, Artificial Sequence
  Synthetic primer
caccgtagaaaatcagtagggactc

SEQ ID NO: 142 - DNA, 25 bp, Artificial Sequence
  Synthetic primer
aaacgagtccctactgattttctac

SEQ ID NO: 143 - DNA, 25 bp, Artificial Sequence
  Synthetic primer
caccgaagttattattactagttag

SEQ ID NO: 144 - DNA, 25 bp, Artificial Sequence
  Synthetic primer
aaacctaactagtaataataacttc

SEQ ID NO: 145 - DNA, 25 bp, Artificial Sequence
  Synthetic primer
caccggaaggatttaaagtctgcac

SEQ ID NO: 146 - DNA, 25 bp, Artificial Sequence
  Synthetic primer
aaacgtgcagactttaaatccttcc

SEQ ID NO: 147 - DNA, 25 bp, Artificial Sequence
  Synthetic primer
caccgatggtacacattttgatggt

SEQ ID NO: 148 - DNA, 25 bp, Artificial Sequence
  Synthetic primer
aaacaccatcaaaatgtgtaccatc

SEQ ID NO: 149 - DNA, 25 bp, Artificial Sequence
  Synthetic primer
caccgtaatatatattccgtcatca

SEQ ID NO: 150 - DNA, 25 bp, Artificial Sequence
  Synthetic primer
aaactgatgacggaatatatattac

SEQ ID NO: 151 - DNA, 25 bp, Artificial Sequence
  Synthetic primer
caccgtatcaatactgttctcacaa

SEQ ID NO: 152 - DNA, 25 bp, Artificial Sequence
  Synthetic primer
aaacttgtgagaacagtattgatac

SEQ ID NO: 153 - DNA, 25 bp, Artificial Sequence
  Synthetic primer
caccgctgttaacttactatttgtc

SEQ ID NO: 154 - DNA, 25 bp, Artificial Sequence
  Synthetic primer
aaacgacaaatagtaagttaacagc

SEQ ID NO: 155 - DNA, 25 bp, Artificial Sequence
  Synthetic primer
caccgtgccccaaagtcaccatatc

SEQ ID NO: 156 - DNA, 25 bp, Artificial Sequence
  Synthetic primer
aaacgatatggtgactttggggcac

SEQ ID NO: 157 - DNA, 25 bp, Artificial Sequence
  Synthetic primer
caccgccagaacctatttcaataac

SEQ ID NO: 158 - DNA, 25 bp, Artificial Sequence
  Synthetic primer
aaacgttattgaaataggttctggc

SEQ ID NO: 159 - DNA, 25 bp, Artificial Sequence
  Synthetic primer
caccgccaacctctcaaaattccct

SEQ ID NO: 160 - DNA, 25 bp, Artificial Sequence
  Synthetic primer
aaacagggaattttgagaggttggc

SEQ ID NO: 161 - DNA, 25 bp, Artificial Sequence
  Synthetic primer
caccgtcgaactgttgattagaggt

SEQ ID NO: 162 - DNA, 25 bp, Artificial Sequence
  Synthetic primer
aaacacctctaatcaacagttcgac

SEQ ID NO: 163 - DNA, 25 bp, Artificial Sequence
  Synthetic primer
caccgggaaacaatgaggacctgac

SEQ ID NO: 164 - DNA, 25 bp, Artificial Sequence
  Synthetic primer
aaacgtcaggtcctcattgtttccc

SEQ ID NO: 165 - DNA, 25 bp, Artificial Sequence
  Synthetic primer
caccgagtgttttaggctaatatag

SEQ ID NO: 166 - DNA, 25 bp, Artificial Sequence
  Synthetic primer
aaacctatattagcctaaaacactc

SEQ ID NO: 167 - DNA, 25 bp, Artificial Sequence
  Synthetic primer
caccgaagagtggtgattaatagat

SEQ ID NO: 168 - DNA, 25 bp, Artificial Sequence
  Synthetic primer
aaacatctattaatcaccactcttc

SEQ ID NO: 169 - DNA, 25 bp, Artificial Sequence
  Synthetic primer
caccggtaggtcagttttaatcaga

SEQ ID NO: 170 - DNA, 25 bp, Artificial Sequence
  Synthetic primer
aaactctgattaaaactgacctacc

SEQ ID NO: 171 - DNA, 25 bp, Artificial Sequence
  Synthetic primer
caccgtatcccctctgagcactgtc

SEQ ID NO: 172 - DNA, 25 bp, Artificial Sequence
  Synthetic primer
aaacgacagtgctcagaggggatac

SEQ ID NO: 173 - DNA, 25 bp, Artificial Sequence
  Synthetic primer
caccgtcaccacaaaaaaagttgga

SEQ ID NO: 174 - DNA, 25 bp, Artificial Sequence
  Synthetic primer
aaactccaactttttttgtggtgac

SEQ ID NO: 175 - DNA, 25 bp, Artificial Sequence
  Synthetic primer
caccgatatgcacttattatttgcc

SEQ ID NO: 176 - DNA, 25 bp, Artificial Sequence
  Synthetic primer
aaacggcaaataataagtgcatatc

SEQ ID NO: 177 - DNA, 25 bp, Artificial Sequence
  Synthetic primer
caccgactattcttattccactgtg

SEQ ID NO: 178 - DNA, 25 bp, Artificial Sequence
  Synthetic primer
aaaccacagtggaataagaatagtc

SEQ ID NO: 179 - DNA, 25 bp, Artificial Sequence
  Synthetic primer
caccgaaggctttactaatttgata

SEQ ID NO: 180 - DNA, 25 bp, Artificial Sequence
  Synthetic primer
aaactatcaaattagtaaagccttc

SEQ ID NO: 181 - DNA, 25 bp, Artificial Sequence
  Synthetic primer
caccgctcgtcaggatattattgct

SEQ ID NO: 182 - DNA, 25 bp, Artificial Sequence
  Synthetic primer
aaacagcaataatatcctgacgagc

SEQ ID NO: 183 - DNA, 25 bp, Artificial Sequence
  Synthetic primer
caccgaaaagagtagacatccccac

SEQ ID NO: 184 - DNA, 25 bp, Artificial Sequence
  Synthetic primer
aaacgtggggatgtctactcttttc

SEQ ID NO: 185 - DNA, 23 bp, Artificial Sequence
  Synthetic primer
agctgggtgtggtggtgagcgcc

SEQ ID NO: 186 - DNA, 34 bp, Artificial Sequence
  Synthetic primer
tcaactttgctatcctcttacatctgtgcctgct

SEQ ID NO: 187 - DNA, 29 bp, Artificial Sequence
  Synthetic primer
tggcagaacttatctaccgccacaggagt

SEQ ID NO: 188 - DNA, 29 bp, Artificial Sequence
  Synthetic primer
agacgaaaaggtttggtggtggctcaagg

SEQ ID NO: 189 - DNA, 22 bp, Artificial Sequence
  Synthetic primer
tgaggatatacaagggcactga

SEQ ID NO: 190 - DNA, 20 bp, Artificial Sequence
  Synthetic primer
agggtacctctgcctctggt

SEQ ID NO: 191 - DNA, 25 bp, Artificial Sequence
  Synthetic primer
agggtggttgggccacctagagaca

SEQ ID NO: 192 - DNA, 22 bp, Artificial Sequence
  Synthetic primer
ggtgagggccagggaaggcccc

SEQ ID NO: 193 - DNA, 32 bp, Artificial Sequence
  Synthetic primer
tgcttcttacaggcagatttccttgggcatca

SEQ ID NO: 194 - DNA, 25 bp, Artificial Sequence
  Synthetic primer
accttccactgtgctcccactgcct

SEQ ID NO: 195 - DNA, 25 bp, Artificial Sequence
  Synthetic primer
tggtgacgagggtacctccaaggca

SEQ ID NO: 196 - DNA, 33 bp, Artificial Sequence
  Synthetic primer
gggcaaagctctacattaggcattttgaggagg

SEQ ID NO: 197 - DNA, 34 bp, Artificial Sequence
  Synthetic primer
acattcctatttgccaaggcagtggagtttttgc

SEQ ID NO: 198 - DNA, 25 bp, Artificial Sequence
  Synthetic primer
agactcttgagggcctgacctcgct

SEQ ID NO: 199 - DNA, 24 bp, Artificial Sequence
  Synthetic primer
aggtgtgccagatgctccacctgt

SEQ ID NO: 200 - DNA, 35 bp, Artificial Sequence
  Synthetic primer
gggatgggaagggaaagaagttgatcttcagttag

SEQ ID NO: 201 - DNA, 25 bp, Artificial Sequence
  Synthetic primer
ctgtgttcactatgcagtgtgagag

SEQ ID NO: 202 - DNA, 25 bp, Artificial Sequence
  Synthetic primer
ctagcagcctaggtatgggtactcg

SEQ ID NO: 203 - DNA, 24 bp, Artificial Sequence
  Synthetic primer
ccagacaactggttaagagagagg

SEQ ID NO: 204 - DNA, 24 bp, Artificial Sequence
  Synthetic primer
agcattactgttcacacaaggcac

SEQ ID NO: 205 - DNA, 27 bp, Artificial Sequence
  Synthetic primer
ctataagaaactggtaaacactgaatg

SEQ ID NO: 206 - DNA, 24 bp, Artificial Sequence
  Synthetic primer
aaatctagggtcgaaagccacagc

SEQ ID NO: 207 - DNA, 20 bp, Artificial Sequence
  Synthetic primer
atcaagcatccagcatttgt

SEQ ID NO: 208 - DNA, 20 bp, Artificial Sequence
  Synthetic primer
gaaacgaagagaggggaagg

SEQ ID NO: 209 - DNA, 20 bp, Artificial Sequence
  Synthetic primer
tcccttaacttgccctgaga

SEQ ID NO: 210 - DNA, 20 bp, Artificial Sequence
  Synthetic primer
aggcacctcagactcagcat

SEQ ID NO: 211 - DNA, 37 bp, Artificial Sequence
  Synthetic primer
ggtttttgagggtgagggtgagggtgagggtgagggt

SEQ ID NO: 212 - DNA, 39 bp, Artificial Sequence
  Synthetic primer
tcccgactatccctatccctatccctatccctatcccta

SEQ ID NO: 213 - DNA, 23 bp, Artificial Sequence
  Synthetic primer
cagcaagtgggaaggtgtaatcc

SEQ ID NO: 214 - DNA, 25 bp, Artificial Sequence
  Synthetic primer
cccattctatcatcaacgggtacaa

SEQ ID NO: 215 - DNA, 20 bp, Artificial Sequence
  Synthetic primer
gcaagaaggtgctgacttcc

SEQ ID NO: 216 - DNA, 20 bp, Artificial Sequence
  Synthetic primer
accatcacgttacccaggag

SEQ ID NO: 217 - DNA, 19 bp, Artificial Sequence
  Synthetic primer
tggatgatctcaagggcac

SEQ ID NO: 218 - DNA, 20 bp, Artificial Sequence
  Synthetic primer
tcagtggtatctggaggaca

SEQ ID NO: 219 - DNA, 21 bp, Artificial Sequence
  Synthetic primer
ctgaggagaagtctgccgtta

SEQ ID NO: 220 - DNA, 20 bp, Artificial Sequence
  Synthetic primer
agcatcaggagtggacagat

SEQ ID NO: 221 - DNA, 22 bp, Artificial Sequence
  Synthetic primer
catgcggaaggatggtattcag

SEQ ID NO: 222 - DNA, 20 bp, Artificial Sequence
  Synthetic primer
ctccccacaattcccgctac

SEQ ID NO: 223 - DNA, 21 bp, Artificial Sequence
  Synthetic primer
gcaacccctactatgccaacc

SEQ ID NO: 224 - DNA, 19 bp, Artificial Sequence
  Synthetic primer
cagtggcgtcttggagaag

SEQ ID NO: 225 - DNA, 20 bp, Artificial Sequence
  Synthetic primer
acccagaagactgtggatgg

SEQ ID NO: 226 - DNA, 20 bp, Artificial Sequence
  Synthetic primer
ttcagctcagggatgacctt

SEQ ID NO: 227 - DNA, 20 bp, Artificial Sequence
  Synthetic primer
tggtgaaggtcggtgtgaac

SEQ ID NO: 228 - DNA, 24 bp, Artificial Sequence
  Synthetic primer
ccatgtagttgaggtcaatgaagg

SEQ ID NO: 229 - DNA, 22 bp, Artificial Sequence
  Synthetic primer
ctcccgaggagtcccaggacat

SEQ ID NO: 230 - DNA, 23 bp, Artificial Sequence
  Synthetic primer
gatggtggtctggctgaacacct

SEQ ID NO: 231 - DNA, 24 bp, Artificial Sequence
  Synthetic primer
aagaaaggagagaagtttggagcc

SEQ ID NO: 232 - DNA, 23 bp, Artificial Sequence
  Synthetic primer
gagatctggcggagaatagttgg

SEQ ID NO: 233 - DNA, 20 bp, Artificial Sequence
  Synthetic primer
ggaagaactgaatctgagcg

SEQ ID NO: 234 - DNA, 18 bp, Artificial Sequence
  Synthetic primer
ctctactggccctggacg

SEQ ID NO: 235 - DNA, 20 bp, Artificial Sequence
  Synthetic primer
atgaaggagccgcaactaga

SEQ ID NO: 236 - DNA, 19 bp, Artificial Sequence
  Synthetic primer
gaggagccaagcaacgagt

SEQ ID NO: 237 - DNA, 19 bp, Artificial Sequence
  Synthetic primer
cggctgcgagagaaattgc

SEQ ID NO: 238 - DNA, 22 bp, Artificial Sequence
  Synthetic primer
ccactttccgttcaaggtcaag

SEQ ID NO: 239 - DNA, 22 bp, Artificial Sequence
  Synthetic primer
cacaagatgaacggcatcaacc

SEQ ID NO: 240 - DNA, 21 bp, Artificial Sequence
  Synthetic primer
cagcgtggtggtggtagtctg

SEQ ID NO: 241 - DNA, 23 bp, Artificial Sequence
  Synthetic primer
ggtctctacagcaagatgaatgg

SEQ ID NO: 242 - DNA, 19 bp, Artificial Sequence
  Synthetic primer
tggcacaggacagtccaag

SEQ ID NO: 243 - DNA, 97 bp, Artificial Sequence
  Synthetic primer
tgctgttgacagtgagcgcccgcaggagctatctcaagattagtgaagccacagatgtaatcttgagatagctcctgcggttgcctactgcctcgga

SEQ ID NO: 244 - DNA, 97 bp, Artificial Sequence
  Synthetic primer
tgctgttgacagtgagcgagcatgtcctatgagccaaatatagtgaagccacagatgtatatttggctcataggacatgcgtgcctactgcctcgga

SEQ ID NO: 245 - DNA, 97 bp, Artificial Sequence
  Synthetic primer
tgctgttgacagtgagcgacgaaagattacctccaaagattagtgaagccacagatgtaatctttggaggtaatctttcgctgcctactgcctcgga

SEQ ID NO: 246 - DNA, 97 bp, Artificial Sequence
  Synthetic primer
tgctgttgacagtgagcgccctcatttcatggagatgaaatagtgaagccacagatgtatttcatctccatgaaatgaggatgcctactgcctcgga

SEQ ID NO: 247 - DNA, 97 bp, Artificial Sequence
  Synthetic primer
tgctgttgacagtgagcgcgggctttggacactattaatatagtgaagccacagatgtatattaatagtgtccaaagcccatgcctactgcctcgga

SEQ ID NO: 248 - DNA, 97 bp, Artificial Sequence
  Synthetic primer
tgctgttgacagtgagcgaccgtactggcaggaaccattatagtgaagccacagatgtataatggttcctgccagtacggctgcctactgcctcgga

SEQ ID NO: 249 - DNA, 97 bp, Artificial Sequence
  Synthetic primer
tgctgttgacagtgagcgaccagtctcccagttatcaaattagtgaagccacagatgtaatttgataactgggagactgggtgcctactgcctcgga

SEQ ID NO: 250 - DNA, 97 bp, Artificial Sequence
  Synthetic primer
tgctgttgacagtgagcgcggttattatgacagaatggattagtgaagccacagatgtaatccattctgtcataataaccatgcctactgcctcgga

SEQ ID NO: 251 - DNA, 97 bp, Artificial Sequence
  Synthetic primer
tgctgttgacagtgagcgcggttgacctgtctgagaaatatagtgaagccacagatgtatatttctcagacaggtcaaccttgcctactgcctcgga

SEQ ID NO: 252 - DNA, 97 bp, Artificial Sequence
  Synthetic primer
tgctgttgacagtgagcgcgctgttagacgcaggaaataatagtgaagccacagatgtattatttcctgcgtctaacagcatgcctactgcctcgga

SEQ ID NO: 253 - DNA, 97 bp, Artificial Sequence
  Synthetic primer
tgctgttgacagtgagcgcccttaggatttggagataaattagtgaagccacagatgtaatttatctccaaatcctaaggttgcctactgcctcgga

SEQ ID NO: 254 - DNA, 97 bp, Artificial Sequence
  Synthetic primer
tgctgttgacagtgagcgacgcaacaagcccagtagtttatagtgaagccacagatgtataaactactgggcttgttgcggtgcctactgcctcgga

SEQ ID NO: 255 - DNA, 97 bp, Artificial Sequence
  Synthetic primer
tgctgttgacagtgagcgagcttgctagttcctatgaaattagtgaagccacagatgtaatttcataggaactagcaagcctgcctactgcctcgga

SEQ ID NO: 256 - DNA, 97 bp, Artificial Sequence
  Synthetic primer
tgctgttgacagtgagcgcccatagaatctcacaccaaattagtgaagccacagatgtaatttggtgtgagattctatggttgcctactgcctcgga

SEQ ID NO: 257 - DNA, 97 bp, Artificial Sequence
  Synthetic primer
tgctgttgacagtgagcgaccagtccaaccagctgataaatagtgaagccacagatgtatttatcagctggttggactgggtgcctactgcctcgga

SEQ ID NO: 258 - DNA, 97 bp, Artificial Sequence
  Synthetic primer
tgctgttgacagtgagcgccctgttaatcgtgattcagaatagtgaagccacagatgtattctgaatcacgattaacaggatgcctactgcctcgga

SEQ ID NO: 259 - DNA, 97 bp, Artificial Sequence
  Synthetic primer
tgctgttgacagtgagcgccctctcccatcctcctcgaaatagtgaagccacagatgtatttcgaggaggatgggagaggttgcctactgcctcgga

SEQ ID NO: 260 - DNA, 97 bp, Artificial Sequence
  Synthetic primer
tgctgttgacagtgagcgaccagatttactcaatgtcaaatagtgaagccacagatgtatttgacattgagtaaatctggctgcctactgcctcgga

SEQ ID NO: 261 - DNA, 97 bp, Artificial Sequence
  Synthetic primer
tgctgttgacagtgagcgcgcagtgcaacacagaattaaatagtgaagccacagatgtatttaattctgtgttgcactgcttgcctactgcctcgga

SEQ ID NO: 262 - DNA, 97 bp, Artificial Sequence
  Synthetic primer
tgctgttgacagtgagcgagcagaaatattgaaaggattatagtgaagccacagatgtataatcctttcaatatttctgcgtgcctactgcctcgga

SEQ ID NO: 263 - DNA, 97 bp, Artificial Sequence
  Synthetic primer
tgctgttgacagtgagcgcccacacatggttaattggaaatagtgaagccacagatgtatttccaattaaccatgtgtggttgcctactgcctcgga

SEQ ID NO: 264 - DNA, 97 bp, Artificial Sequence
  Synthetic primer
tgctgttgacagtgagcgcgggcagaaatggatctggaaatagtgaagccacagatgtatttccagatccatttctgcccatgcctactgcctcgga

SEQ ID NO: 265 - DNA, 97 bp, Artificial Sequence
  Synthetic primer
tgctgttgacagtgagcgcggccctaaaggaactggatattagtgaagccacagatgtaatatccagttcctttagggccatgcctactgcctcgga

SEQ ID NO: 266 - DNA, 97 bp, Artificial Sequence
  Synthetic primer
tgctgttgacagtgagcgacctgaagtatctgtatccaaatagtgaagccacagatgtatttggatacagatacttcagggtgcctactgcctcgga

SEQ ID NO: 267 - DNA, 97 bp, Artificial Sequence
  Synthetic primer
tgctgttgacagtgagcgcgcaagcgtaatcttcaggatatagtgaagccacagatgtatatcctgaagattacgcttgcttgcctactgcctcgga

SEQ ID NO: 268 - DNA, 97 bp, Artificial Sequence
  Synthetic primer
tgctgttgacagtgagcgccagctgttactcaagaagatgtagtgaagccacagatgtacatcttcttgagtaacagctgttgcctactgcctcgga

SEQ ID NO: 269 - DNA, 97 bp, Artificial Sequence
  Synthetic primer
tgctgttgacagtgagcgaggcagcataaaggccctatattagtgaagccacagatgtaatatagggcctttatgctgccctgcctactgcctcgga

SEQ ID NO: 270 - DNA, 97 bp, Artificial Sequence
  Synthetic primer
tgctgttgacagtgagcgcggagcacatactatagcaaattagtgaagccacagatgtaatttgctatagtatgtgctccatgcctactgcctcgga

SEQ ID NO: 271 - DNA, 97 bp, Artificial Sequence
  Synthetic primer
tgctgttgacagtgagcgcccatgatttgcaagcacataatagtgaagccacagatgtattatgtgcttgcaaatcatggatgcctactgcctcgga

SEQ ID NO: 272 - DNA, 57 bp, Artificial Sequence
  Synthetic primer
ccggcaacaagatgaagagcaccaactcgagttggtgctcttcatcttgttgttttt

SEQ ID NO: 273 - DNA, 58 bp, Artificial Sequence
  Synthetic primer
ccggcgcacagaacactcatggattctcgagaatccatgagtgttctgtgcgtttttg

SEQ ID NO: 274 - DNA, 58 bp, Artificial Sequence
  Synthetic primer
ccggccagaggatgacgattgtttactcgagtaaacaatcgtcatcctctggtttttg

SEQ ID NO: 275 - DNA, 58 bp, Artificial Sequence
  Synthetic primer
ccgggcatagacgatggcactgttactcgagtaacagtgccatcgtctatgctttttg

SEQ ID NO: 276 - DNA, 57 bp, Artificial Sequence
  Synthetic primer
ccgggctgacacagttattatctatctcgagatagataataactgtgtcagcttttt

SEQ ID NO: 277 - DNA, 57 bp, Artificial Sequence
  Synthetic primer
ccgggcgggagttcagtaccaataactcgagttattggtactgaactcccgcttttt

SEQ ID NO: 278 - DNA, 57 bp, Artificial Sequence
  Synthetic primer
ccgggcaaactttatgtttgggattctcgagaatcccaaacataaagtttgcttttt

SEQ ID NO: 279 - DNA, 57 bp, Artificial Sequence
  Synthetic primer
ccggccagagacatacataggaattctcgagaattcctatgtatgtctctggttttt

SEQ ID NO: 280 - DNA, 57 bp, Artificial Sequence
  Synthetic primer
ccgggcagcattcttatagctgtttctcgagaaacagctataagaatgctgcttttt

SEQ ID NO: 281 - DNA, 57 bp, Artificial Sequence
  Synthetic primer
ccggcctataacaatgcagtgtatactcgagtatacactgcattgttataggttttt

SEQ ID NO: 282 - DNA, 57 bp, Artificial Sequence
  Synthetic primer
ccggccagtgaatctaatgtgactactcgagtagtcacattagattcactggttttt

SEQ ID NO: 283 - DNA, 57 bp, Artificial Sequence
  Synthetic primer
ccggcgttcttaactttgaaccatactcgagtatggttcaaagttaagaacgttttt

SEQ ID NO: 284 - DNA, 57 bp, Artificial Sequence
  Synthetic primer
ccgggccggtcatgtccaaagtaatctcgagattactttggacatgaccggcttttt

SEQ ID NO: 285 - DNA, 57 bp, Artificial Sequence
  Synthetic primer
ccgggctgctcaactatggtctctactcgagtagagaccatagttgagcagcttttt

SEQ ID NO: 286 - DNA, 57 bp, Artificial Sequence
  Synthetic primer
ccgggcagactcattatctggtgatctcgagatcaccagataatgagtctgcttttt

SEQ ID NO: 287 - DNA, 57 bp, Artificial Sequence
  Synthetic primer
ccgggcaaatactatgctgtcaattctcgagaattgacagcatagtatttgcttttt

SEQ ID NO: 288 - DNA, 57 bp, Artificial Sequence
  Synthetic primer
ccggcagtctcaccaatttcagaaactcgagtttctgaaattggtgagactgttttt

SEQ ID NO: 289 - DNA, 57 bp, Artificial Sequence
  Synthetic primer
ccggccagcgtttgatggactctttctcgagaaagagtccatcaaacgctggttttt

SEQ ID NO: 290 - DNA, 58 bp, Artificial Sequence
  Synthetic primer
ccgggcggaggatttacgaatgcttctcgagaagcattcgtaaatcctccgctttttg

SEQ ID NO: 291 - DNA, 58 bp, Artificial Sequence
  Synthetic primer
ccggccgttggtaaacctcacaaatctcgagatttgtgaggtttaccaacggtttttg

SEQ ID NO: 292 - DNA, 58 bp, Artificial Sequence
  Synthetic primer
ccgggccgaagctataaacagcgaactcgagttcgctgtttatagcttcggctttttg

SEQ ID NO: 293 - DNA, 58 bp, Artificial Sequence
  Synthetic primer
ccggcgccaaacgtaagagctctatctcgagatagagctcttacgtttggcgtttttg

SEQ ID NO: 294 - DNA, 57 bp, Artificial Sequence
  Synthetic primer
ccggcccaagatgtataatgcttatctcgagataagcattatacatcttgggttttt

SEQ ID NO: 295 - DNA, 57 bp, Artificial Sequence
  Synthetic primer
ccggccctaataactgttcaggaaactcgagtttcctgaacagttattagggttttt

SEQ ID NO: 296 - DNA, 57 bp, Artificial Sequence
  Synthetic primer
ccgggctggtatttagaccgatcatctcgagatgatcggtctaaataccagcttttt

SEQ ID NO: 297 - DNA, 57 bp, Artificial Sequence
  Synthetic primer
ccgggctttgattgtgaagcatttactcgagtaaatgcttcacaatcaaagcttttt

SEQ ID NO: 298 - DNA, 57 bp, Artificial Sequence
  Synthetic primer
ccggccatacgtttaacagcacaatctcgagattgtgctgttaaacgtatggttttt

SEQ ID NO: 299 - DNA, 57 bp, Artificial Sequence
  Synthetic primer
ccggccctagttcatcgcaacctttctcgagaaaggttgcgatgaactagggttttt

SEQ ID NO: 300 - DNA, 57 bp, Artificial Sequence
  Synthetic primer
ccgggcgatctttgtagaatttgatctcgagatcaaattctacaaagatcgcttttt

SEQ ID NO: 301 - DNA, 57 bp, Artificial Sequence
  Synthetic primer
ccgggctgttaatgtgatggtgtatctcgagatacaccatcacattaacagcttttt

SEQ ID NO: 302 - DNA, 57 bp, Artificial Sequence
  Synthetic primer
ccggccttgtagataaactgggtttctcgagaaacccagtttatctacaaggttttt

SEQ ID NO: 303 - DNA, 58 bp, Artificial Sequence
  Synthetic primer
ccggtgcacatgaagcgccacctttctcgagaaaggtggcgcttcatgtgcatttttg

SEQ ID NO: 304 - DNA, 58 bp, Artificial Sequence
  Synthetic primer
ccggccctccttcctgagttgtttgctcgagcaaacaactcaggaaggagggtttttg

SEQ ID NO: 305 - DNA, 58 bp, Artificial Sequence
  Synthetic primer
ccggcagaggatccaggtgtgatagctcgagctatcacacctggatcctctgtttttg

SEQ ID NO: 306 - DNA, 58 bp, Artificial Sequence
  Synthetic primer
ccggcgcagtattgtccaaattattctcgagaataatttggacaatactgcgtttttg

SEQ ID NO: 307 - DNA, 58 bp, Artificial Sequence
  Synthetic primer
ccgggccatcaaacacaatgtcaaactcgagtttgacattgtgtttgatggctttttg

SEQ ID NO: 308 - DNA, 58 bp, Artificial Sequence
  Synthetic primer
ccgggctctcaaagttcagactcttctcgagaagagtctgaactttgagagctttttg

SEQ ID NO: 309 - DNA, 58 bp, Artificial Sequence
  Synthetic primer
ccggcctctattactacctgactaactcgagttagtcaggtagtaatagaggtttttg

SEQ ID NO: 310 - DNA, 58 bp, Artificial Sequence
  Synthetic primer
ccgggcagtgtaagaacttctacttctcgagaagtagaagttcttacactgctttttg

SEQ ID NO: 311 - DNA, 58 bp, Artificial Sequence
  Synthetic primer
ccggcccttgtatcatcgcaacaaactcgagtttgttgcgatgatacaagggtttttg

SEQ ID NO: 312 - DNA, 58 bp, Artificial Sequence
  Synthetic primer
ccgggcctttctttcaatccttatactcgagtataaggattgaaagaaaggctttttg

SEQ ID NO: 313 - DNA, 58 bp, Artificial Sequence
  Synthetic primer
ccggcggcagtagtagaagatgtttctcgagaaacatcttctactactgccgtttttg

SEQ ID NO: 314 - DNA, 58 bp, Artificial Sequence
  Synthetic primer
ccgggcagactgaatgtctgggattctcgagaatcccagacattcagtctgctttttg

SEQ ID NO: 315 - DNA, 57 bp, Artificial Sequence
  Synthetic primer
ccggccatatttatacagcagagaactcgagttctctgctgtataaatatggttttt

SEQ ID NO: 316 - DNA, 57 bp, Artificial Sequence
  Synthetic primer
ccggcccgtggacttcaagaagatactcgagtatcttcttgaagtccacgggttttt

SEQ ID NO: 317 - DNA, 57 bp, Artificial Sequence
  Synthetic primer
ccggccgaggtctgatagtgaagaactcgagttcttcactatcagacctcggttttt

SEQ ID NO: 318 - DNA, 57 bp, Artificial Sequence
  Synthetic primer
ccggcggcagacactgtgatcatttctcgagaaatgatcacagtgtctgccgttttt

SEQ ID NO: 319 - DNA, 57 bp, Artificial Sequence
  Synthetic primer
ccgggcaggatattagctccttatactcgagtataaggagctaatatcctgcttttt

SEQ ID NO: 320 - DNA, 57 bp, Artificial Sequence
  Synthetic primer
ccggcccaccacatttacccatattctcgagaatatgggtaaatgtggtgggttttt

SEQ ID NO: 321 - DNA, 57 bp, Artificial Sequence
  Synthetic primer
ccgggctatgatacttgggtccatactcgagtatggacccaagtatcatagcttttt

SEQ ID NO: 322 - DNA, 57 bp, Artificial Sequence
  Synthetic primer
ccggcctagctgtttatcgacggaactcgagttccgtcgataaacagctaggttttt

SEQ ID NO: 323 - DNA, 58 bp, Artificial Sequence
  Synthetic primer
ccgggcttacgtttactggtttcttctcgagaagaaaccagtaaacgtaagctttttg

SEQ ID NO: 324 - DNA, 58 bp, Artificial Sequence
  Synthetic primer
ccggccaaacctcttgccactagaactcgagttctagtggcaagaggtttggtttttg

SEQ ID NO: 325 - DNA, 58 bp, Artificial Sequence
  Synthetic primer
ccggcggaatctcatagcaccaatactcgagtattggtgctatgagattccgtttttg

SEQ ID NO: 326 - DNA, 58 bp, Artificial Sequence
  Synthetic primer
ccgggctgacaatcaaatgaatcatctcgagatgattcatttgattgtcagctttttg

SEQ ID NO: 327 - DNA, 57 bp, Artificial Sequence
  Synthetic primer
ccggcctggctctgttctctgtcctctcgagaggacagagaacagagccaggttttt

SEQ ID NO: 328 - DNA, 57 bp, Artificial Sequence
  Synthetic primer
ccggctgagaccaaacctgtgcttactcgagtaagcacaggtttggtctcagttttt

SEQ ID NO: 329 - DNA, 57 bp, Artificial Sequence
  Synthetic primer
ccggcccttccatgttagcactttactcgagtaaagtgctaacatggaagggttttt

SEQ ID NO: 330 - DNA, 57 bp, Artificial Sequence
  Synthetic primer
ccggcggtgaagatacttatgatatctcgagatatcataagtatcttcaccgttttt

SEQ ID NO: 331 - protein, 9 aa, Artificial Sequence
  Synthetic peptide
MDYKDDDDK

SEQ ID NO: 332 - protein, 15 aa, Artificial Sequence
  Synthetic peptide
SGLNDIFEAQKIEWH

SEQ ID NO: 333 - DNA, 4239 bp, Artificial Sequence
  Synthetic primer
ggcctgaacgacatcttcgaggctcagaaaatcgaatggcacgaaggcgcgccgagctcgaggatccttgctagccccaaaaagaagaggaaagtggacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttcctaa

SEQ ID NO: 334 - protein, 1412 aa, Artificial Sequence
  Synthetic peptide
GLNDIFEAQKIEWHEGAPSSRILASPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSS